SEQUENCE LISTING (11 sequences)

SEQ ID NO: 1 - protein, 5 aa, synthetic construct
DYYIH

SEQ ID NO: 2 - protein, 17 aa, synthetic construct
RVNPDHGDSYYNQKFKD

SEQ ID NO: 3 - protein, 6 aa, synthetic construct
NYLFDH

SEQ ID NO: 4 - protein, 11 aa, synthetic construct
KASQDVGTAVA

SEQ ID NO: 5 - protein, 7 aa, synthetic construct
WASIRHT

SEQ ID NO: 6 - protein, 7 aa, synthetic construct
HQFATYT

SEQ ID NO: 7 - protein, 115 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 8 - protein, 105 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 9 - protein, 444 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 212 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 9 aa, synthetic construct
ARNYLFDHW

(19) United States
(12) Patent Application Publication
(10) Pub. No.: US 2026/0248947 A1
(43) Pub. Date: Aug. 27, 2026

(54) Methods of Treating Cancer Using Anti-HER2 Antibody-Drug Conjugates and HER2 Kinase Inhibitors

(71) Applicant: Seagen Inc., Bothell, WA (US)

(72) Inventors: Anita Kulukian, Seattle, WA (US); Amy Hyonju Kim, Emerald Hills, CA (US); Nicholas Wei Wen Choong, Belmont, CA (US)

(73) Assignee: Seagen Inc., Bothell, WA (US)

(21) Appl. No.: 19/163,450

(22) PCT Filed: Mar. 8, 2024
(86) PCT No.: PCT/US24/19136

§ 371 Date: Sep. 9, 2025

Related U.S. Application Data
(60) Provisional application No. 63/489,703, filed on Mar. 10, 2023.
(60) Provisional application No. 63/602,262, filed on Nov. 22, 2023.

(51) Int. Cl. A61K 47/68; A61K 31/517; A61P 35/00

(52) U.S. Cl. CPC A61K 47/6851; A61K 31/517; A61K 47/6803; A61K 47/68031; A61K 47/68035; A61K 47/68037; A61K 47/6805; A61K 47/6809; A61K 47/6889; A61P 35/00

(57) ABSTRACT

Provided herein are methods for treating or preventing progression of cancer in an individual, comprising administering to the individual an effective amount of tucatinib and an anti-human epidermal growth factor receptor 2 (HER2) antibody-drug conjugate that comprises an anti-HER2 antibody and a cytotoxic molecule. Compositions, uses, and kits related thereto are also provided.

### CROSS-REFERENCE TO RELATED APPLICATIONS

This application claims priority to U.S. Provisional Patent Application No. 63/489,703, filed Mar. 10, 2023, and U.S. Provisional Patent Application No. 63/602,262, filed Nov. 22, 2023, which are herein incorporated by reference in their entirety for all purposes.

### REFERENCE TO AN ELECTRONIC SEQUENCE LISTING

The content of the electronic sequence listing (761682010140seglist.xml; Size: 11,210 bytes; and Date of Creation: Mar. 4, 2024) is herein incorporated by reference in its entirety.

### FIELD

The present disclosure relates to methods for treating or preventing progression of cancer in an individual, as well as compositions, uses, and kits related thereto. In some embodiments, the methods comprise administering to the individual an effective amount of tucatinib and an anti-human epidermal growth factor receptor 2 (HER2) antibody-drug conjugate that comprises an anti-HER2 antibody and a cytotoxic molecule.

### BACKGROUND

ErbB2, also known as HER2/neu, is a member of a family of tyrosine kinases that regulate cell growth and survival (Lengyel, C. G. et al. (2021) Gastrointest Disord 3(1):1-22). Overexpression and/or amplification of HER2 is seen in many malignancies including breast, gastric, ovarian, pancreatic, colorectal, and endometrial cancer (Neve, R. M. et al. (2001) Ann Oncol 12(Suppl1):S9-S13; Menard, S. et al. (2003) Oncogene 22(42):6570-6578; Moasser, M. M. (2007) Oncogene 26(45):6469-6487; Iqbal, N. et al. (2014) Mol Biol Int 2014:852748). Tumors that overexpress HER2 are thought to be more aggressive and associated with poorer overall survival (OS) compared to HER2-negative cancers. Cancers characterized by overexpression of HER2 (e.g., HER2-positive cancers) are often correlated with poor prognosis and/or are resistant to many standard therapies. As such, a handful of therapies targeting HER2 have been approved for treatment of HER2-positive cancer, including antibodies such as trastuzumab and pertuzumab, antibody-drug conjugates (ADCs) such as trastuzumab maytansine (T-DM 1), and small molecule tyrosine kinase inhibitors (TKIs) such as lapatinib, pazopanib, afatinib, and neratinib. Oncologists have traditionally applied a binary means of treatment decision making when considering HER2 (either HER2+ or HER2-negative), as until recently the benefit of anti-HER2 targeted therapies were limited to patients whose breast cancer is HER2+.

The introduction of HER2-targeted therapy using either antibody-based therapies or small molecule tyrosine kinase inhibitors (TKI) has led to significant and ongoing improvements in disease-free survival (DFS), progression-free survival (PFS), and OS in both the neoadjuvant/adjuvant and metastatic settings (Slamon, D. J. et al. (2001) N Engl J Med 344(11):783-792; Geyer, C. E. et al. (2006) N Engl J Med 355(26):2733-2743; Baselga 2012; Verma 2012). The standard of care for most patients with HER2+ locally advanced or metastatic breast cancer (LA/mBC) in the first-line (1L) setting is pertuzumab plus trastuzumab and a taxane, based on results of the pivotal CLEOPATRA study (Baselga, J. et al. (2012) N Engl J Med 366(2):109-119; Giordano, S. H. et al. (2014) J Clin Oncol 32(19):2078-2099). The CLEOPATRA study and the subsequent open-label single-arm 1L PERUSE study also established the pattern of dropping taxane after an “induction” phase and continuing mAb therapy in a “maintenance” phase (Bachelot, T. et al. (2019) Ann Oncol 30(5):766-773; Miles, D. et al. (2017) Ann Oncol 28(11):2761-2767). However, ultimately most patients experienced progression on maintenance therapy.

For those patients that progress, the standard 2L treatment is the HER2-targeting ADC trastuzumab emtansine (T-DM1) as established in the EMILIA phase 3 trial (Verma, S. et al. (2012) N Engl J Med 367(19):1783-1791). However, recent trials have provided more options for subjects in the 2L setting. The HER2CLIM1B study that evaluated the combination of the TKI tucatinib plus trastuzumab and capecitabine compared to trastuzumab and capecitabine control showed significant improvements for patients that progressed following anti-HER2-based therapy, particularly those with brain metastases (Lin, N. U. et al. (2020) J Clin Oncol 38(23):2610-2619; Murthy, R. K. et al. (2020) N Engl J Med 382(7):597-609). The recent DESTINY-Breast03 trial compared T-DM1 with the new ADC trastuzumab deruxtecan (T-DXd) in patients that progressed following taxane plus trastuzumab treatment (Cortes, J. et al. (2022) N Engl J Med 386(12):1143-1154). An overall response (either complete or partial response) occurred in 79.7% (95% CI: 74.3%-84.4%) of subjects who received T-DXd compared to 34.2% (95% CI: 28.5-40.3%) of subjects who received T-DM1. Of subjects who received T-DXd, 75.8% were alive without disease progression at 12 months (95% CI: 69.8%-8.7%) compared to 34.1% (95% CI: 27.7%-40.5%) of subjects who received T-DM1. Combining anti-HER2 antibodies with anti-HER2 TKIs has been proposed as a combination treatment for cancer (see also International Pub. No. WO2021/097220 and WO2022/067347), but the efficacy and safety profiles for many such combinations have not been explored. Evaluating further combinations of TKIs with HER2-targeting ADCs is important, particularly for patients with LA/mBC in 3L or higher settings who already face fewer treatment options.

Breast cancers with immunohistochemistry (IHC) 2+/in situ hybridization (ISH)-negative and IHC 1+ are traditionally categorized with IHC 0 as HER2-negative tumors and are considered ineligible for anti-HER2 therapies despite displaying similarities to HER2+ tumors (eg, larger tumor size, higher histopathological grade, higher Ki67, and frequent axillary nodal involvement) (Mendes, D. et al. (2015) Breast Cancer Res 17:140; Cardoso, F. et al. (2018) Ann Oncol 29(8):1634-1657; Cardoso, F. et al. (2019) Ann Oncol 30(8):1194-1220; Eiger, D. et al. (2021) Cancers (Basel) 13(5):1015). HER2-low is an emerging nomenclature to describe the IHC 2+/ISH-negative and IHC 1+ subgroup of breast cancer and is estimated to comprise about 60% of HER2-negative breast cancer (Schettini, F. et al. (2021) NPJ Breast Cancer 7(1):1). Because these patients with HER2-low breast cancer were not found to benefit from anti-HER2 targeted therapies until recently (Modi, S. et al. (2022) N Engl J Med 387(1):9-20; Rugo, H. S. et al. (2022) J Clin Oncol 40:Abstract LBA1001), treatment for this patient population focused on other relevant tumor markers (eg, ER). As a result, treatment according to 2 traditional breast cancer categories has become common: (1) HR+/HER2-negative breast cancer, and (2) triple-negative breast cancer (TNBC), which tumors do not express ER, PR, or HER2. Historically, patients with HR+/HER2-negative LA/mBC are managed initially with hormonal therapy with or without targeted therapy through multiple sequential lines. The targeted treatment options generally end upon failure of hormonal therapies. Recently, the DESTINY-Breast04 trial showed that targeting HER2 provides clinically meaningful benefits for patients with HER2-low metastatic breast cancer. T-DXd improved median progression-free survival by 4.8 months and median overall survival by 6.6 months compared with standard single-agent chemotherapy in this heavily pretreated patient population. Data established a new standard of care for patients with HER2-low metastatic breast cancer (Modi, S. et al. (2022) N Engl J Med 387(1):9-20). As a result, T-DXd was approved by FDA as the first HER2 targeted therapy for HER2-low LA/mBC patients. Recent data on other therapy such as sacituzumab govitecan, has shown favorable results in HER2-negative LA/mBC. The TROPiCS-02 study was a phase 3 study investigating the efficacy of sacituzumab govitecan in HR+/IER2-negative locally recurrent inoperable or metastatic breast cancer with prior endocrine therapy, CDK 4/6 inhibitor, and at least 2 lines of chemotherapy in the metastatic setting (Rugo, H. S. et al. (2022) J Cin Oncol 40:Abstract LBA1001). Available clinical data demonstrated that HER2-targeted therapies are a viable approach to treatment in HER2-low LA/mBC disease.

Combined with encouraging preclinical and clinical data on combining HER2-targeting agents with a TKI, pursuing additional HER2-targeted therapies for HER2-low LA/mBC populations would be advantageous. As such, there remains a need for safe and effective HER2-targeted combination therapies, particularly for treatment of HER2-low LA/mBC.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

In some aspects, provided herein are methods for treating or preventing progression of cancer in an individual, comprising administering to the individual an effective amount of tucatinib and an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); and wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

In some aspects, provided herein are methods for treating or preventing progression of cancer in an individual, comprising administering to the individual an effective amount of tucatinib and an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:11); and wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

In some embodiments according to any of the embodiments described herein, the VH domain of the anti-HER2 antibody comprises the amino acid sequence of SEQ ID NO:7 and/or the VL domain of the anti-HER2 antibody comprises the amino acid sequence of SEQ ID NO:8. In some embodiments, the VH domain of the anti-HER2 antibody comprises the amino acid sequence of SEQ ID NO:7, and the VL domain of the anti-HER2 antibody comprises the amino acid sequence of SEQ ID NO:8. In some embodiments, the heavy chain of the anti-HER2 antibody comprises the amino acid sequence of SEQ ID NO:9 and/or the light chain of the anti-HER2 antibody comprises the amino acid sequence of SEQ ID NO:10. In some embodiments, the heavy chain of the anti-HER2 antibody comprises the amino acid sequence of SEQ ID NO:9, and the light chain of the anti-HER2 antibody comprises the amino acid sequence of SEQ ID NO:10.

In some embodiments according to any of the embodiments described herein, the cytotoxic molecule comprises a tubulin inhibitor or DNA damaging agent. In some embodiments, the tubulin inhibitor comprises a dolastatin or derivative thereof, auristatin or derivative thereof, or maytansinoid or derivative thereof. In some embodiments, the tubulin inhibitor comprises monomethyl auristatin E (MMAE), monomethyl auristatin F (MMAF), or auristatin F (AF). In some embodiments, the tubulin inhibitor comprises emtansine (DM1), maytansine (DM3), or ravtansine (DM4). In some embodiments, the DNA damaging agent comprises a calicheamicin, duocarmycin, pyrrolobenzodiazepine (PBD), or SN-38. In some embodiments, the cytotoxic molecule comprises an amanitin, anthracycline, baccatin, camptothecin, cemadotin, colchicine, colcimid, combretastatin, cryptophycin, dicodermolide, docetaxel, doxorubicin, echinomycin, eleutherobin, epothilone, estramustine, lexitropsin, maytansine, methotrexate, netropsin, puromycin, rhizoxins, taxane, tubulysin, or vinca alkaloid. In some embodiments, the antibody-drug conjugate is represented by formula Ab-(L-U)n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody. In some embodiments, the number of cytotoxic molecules bound to the antibody refers to an average number of cytotoxic molecules bound to the antibody, e.g., in a sample, preparation, or composition. In some embodiments, the linker is attached to the anti-HER2 antibody via a thiol or amino moiety. In some embodiments, the linker is selected from the group consisting of maleimidocaproyl valine citrulline p-amino-benzyloxy (mc-vc-pAB), maleimidocaproyl (mc), triglycyl peptide linker, 3-maleimido-propionic acid, Mal-di-EG-OPFP (perfluorophenyl 3-(2-(2-(3-(2,5-dioxo-2,5-dihydro-1H-pyrrol-1-yl)propanamido)ethoxy)ethoxy)propanoate), Mal-di-EG-OSu (2,5-dioxopyrrolidin-1-yl 3-(2-(2-(2,5-dioxo-2,5-dihydro-1H-pyrrol-1-yl)ethoxy)ethoxy)propanoate), Mal-Tri-EG-OSu (2,5-dioxopyrrolidin-1-yl 3-(2-(2-(2-(2,5-dioxo-2,5-dihydro-1H-pyrrol-1-yl)ethoxy)ethoxy)ethoxy)ethoxypropanoate), Mal-Tetra-EG-OSu (2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate), Br-di-EG-OSu (2,5-dioxopyrrolidin-1-yl 3 (2-(2-(2-bromoacetamido)ethoxy)ethoxy)propanoate), Py-ds-prp-OSu (2-5-dioxopyrrolidin-1-yl 3-(pyridine-2-yldisulfanyl)propanoate), Py-ds-Prp-OPEP (perfluorophenyl 3-(pyridine-2-yldisulfanyl)propanoate), Py-ds-dmBut-OSu (2,5-dioxopyrrolidin-1-yl 4-methyl-4-(pyridine-2-yldisulfanyl)pentanoate, Py-ds-dmBut-OPF (perfluorophenyl 4-methyl-4-(pyridine-2-yldisulfanyl)pentanoate), SMCC (N-succinimidyl 4-(maleimidomethyl) cyclohexanecarboxylate), MBS (3-maleimidobenzoic acid N-hydroxysuccinimide ester), SATA (S—(N-succinimidyl)thioacetate), SPDP ((N-succinimidyl 3-(2-pyridyldithio)propionate), and SMPT ((N-succinimidyloxy carbonyl)-1-methyl-1-(2-pyridyldithio)toluene). In some embodiments, the cytotoxic molecule comprises MMAE, and the linker is a maleimidocaproyl valine citrulline p-amino-benzyloxy (mc-vc-pAB) linker. In some embodiments, the cytotoxic molecule comprises MMAE, the linker is a maleimidocaproyl valine citrulline p-amino-benzyloxy (mc-vc-pAB) linker, and the anti-HER2 antibody comprises a heavy chain comprising the amino acid sequence of SEQ ID NO:9 and a light chain comprising the amino acid sequence of SEQ ID NO:10. In some embodiments, an average of 4 cytotoxic molecules are conjugated to the anti-HER2 antibody. In some embodiments, the antibody-drug conjugate is disitamab vedotin.

In some embodiments according to any of the embodiments described herein, the cancer has previously been treated. In some embodiments, prior to administration of tucatinib and the antibody-drug conjugate, the individual has experienced progression on or after one or more standard of care therapies. In some embodiments, the individual is intolerant to one or more standard of care therapies. In some embodiments, cells of the cancer express HER2.

In some embodiments according to any of the embodiments described herein, the cancer is breast cancer. In some embodiments, the cancer is locally advanced or metastatic breast cancer (LA/mBC). In some embodiments, administration of tucatinib and the antibody-drug conjugate is a second-line (2L) treatment or a third-line or higher (3L+) treatment. In some embodiments, the cancer is a HER2-low cancer, e.g., HER2-low breast cancer or HER2-low LA/mBC. In some embodiments, a sample obtained from the individual comprises cancer cells that express HER2 on their cell surface at a level of IHC1+, as measured by immunohistochemistry (IHC) assay. In some embodiments, a sample obtained from the individual comprises cancer cells that express HER2 on their cell surface at a level of IHC2+, as measured by IHC assay; and a sample obtained from the individual comprises cancer cells that do not exhibit HER2 gene amplification, as measured by in situ hybridization (ISH) assay (ISH-negative). In some embodiments, the cancer is a HER2-positive cancer, e.g., HER2-positive breast cancer or HER2-positive LA/mBC. In some embodiments, a sample obtained from the individual comprises cancer cells that express HER2 on their cell surface at a level of IHC3+, as measured by IHC assay. In some embodiments, a sample obtained from the individual comprises cancer cells that express HER2 on their cell surface at a level of IHC2+, as measured by IHC assay; and a sample obtained from the individual comprises cancer cells that exhibit HER2 gene amplification, as measured by ISH assay (ISH-positive). In some embodiments, the cancer is HER2-positive LA/mBC; and wherein the administration of tucatinib and the antibody-drug conjugate is a third-line or higher (3L+) treatment. In some embodiments, the individual has a visceral organ metastasis. In some embodiments, the individual does not have a visceral organ metastasis. In some embodiments, the cancer is HER2-low LA/mBC; and wherein the administration of tucatinib and the antibody-drug conjugate is a second-line (2L) treatment or a third-line or higher (3L+) treatment. In some embodiments, a sample obtained from the individual comprises cancer cells that express HER2 on their cell surface at a level of IHC1+, as measured by immunohistochemistry (IHC) assay. In some embodiments, a sample obtained from the individual comprises cancer cells that express HER2 on their cell surface at a level of IHC2+, as measured by IHC assay; and a sample obtained from the individual comprises cancer cells that do not exhibit HER2 gene amplification, as measured by in situ hybridization (ISH) assay (ISH-negative). In some embodiments, e.g., prior to administration of a treatment as disclosed herein, the individual has not been treated with a HER2-directed therapy. In some embodiments, e.g., for treatment of HER2-positive breast cancer, the individual has been previously treated with trastuzumab with or without pertuzumab (e.g., prior to administration of a treatment as disclosed herein). In some embodiments, e.g., for treatment of HER2-positive breast cancer, the individual has experienced progression on or after treatment with T-DXd or other topoisomerase I inhibitor directed therapy; or the individual is intolerant to treatment with T-DXd or other topoisomerase I inhibitor directed therapy. In some embodiments, e.g., for treatment of HER2-positive breast cancer, the individual has received no more than 3 prior systemic cytotoxic chemotherapy regimens. In some embodiments, e.g., for treatment of HER2-positive breast cancer, the individual has been previously treated with PARP-inhibitor (e.g., prior to administration of a treatment as disclosed herein), and the cancer comprises cells having a mutation in a BRCA gene, e.g., BRCA1 or BRCA2.

In some embodiments according to any of the embodiments described herein, the cancer is gastric cancer or gastroesophageal junction cancer (GEJC). In some embodiments, the cancer is gastric adenocarcinoma or gastroesophageal junction adenocarcinoma. In some embodiments, the cancer is locally advanced or metastatic gastric cancer or GEJC (LA/mGC/GEJC). In some embodiments, the administration of tucatinib and the antibody-drug conjugate is a second-line (2L) treatment. In some embodiments, the cancer is a HER2-positive cancer. In some embodiments, a sample obtained from the individual comprises cancer cells that express HER2 on their cell surface at a level of IHC3+, as measured by IHC assay. In some embodiments, a sample obtained from the individual comprises cancer cells that express HER2 on their cell surface at a level of IHC2+, as measured by IHC assay; and a sample obtained from the individual comprises cancer cells that exhibit HER2 gene amplification, as measured by ISH assay (ISH-positive).

In some embodiments, the cancer is a HER2-low cancer, e.g., HER2-low gastric cancer or GEJC, HER2-low gastric adenocarcinoma or gastroesophageal junction adenocarcinoma, or HER2-low LA/mGC/GEJC. In some embodiments, the cancer is HER2-low LA/mGC/GEJC. In some embodiments, a sample obtained from the individual comprises cancer cells that express HER2 on their cell surface at a level of IHC1+, as measured by IHC assay. In some embodiments, a sample obtained from the individual comprises cancer cells that express HER2 on their cell surface at a level of IHC2+, as measured by IHC assay; and a sample obtained from the individual comprises cancer cells that do not exhibit HER2 gene amplification, as measured by in situ hybridization (ISH) assay (ISH-negative). In some embodiments, e.g., for treatment of HER2-low gastric or gastroesophageal junction adenocarcinoma, the individual has been treated with a prior systemic therapy comprising a platinum-containing drug, fluorouracil (FU), or taxane (e.g., prior to administration of a treatment as disclosed herein). In some embodiments, e.g., prior to administration of a treatment as disclosed herein, the individual has not been treated with a HER2-directed therapy.

In some embodiments according to any of the embodiments described herein, the antibody-drug conjugate is administered to the individual at a dose of 1.0 mg/kg, 1.25 mg/kg, or 1.5 mg/kg. In some embodiments, the antibody-drug conjugate is administered to the individual at a dose of 1.0 mg/kg. In some embodiments, the antibody-drug conjugate is administered to the individual at a dose of 1.25 mg/kg. In some embodiments, the antibody-drug conjugate is administered to the individual at a dose of 1.5 mg/kg. In some embodiments, the antibody-drug conjugate is administered to the individual with a maximum dose of 150 mg. In some embodiments, the antibody-drug conjugate is administered intravenously to the individual. In some embodiments, the antibody-drug conjugate is administered to the individual every 2 weeks or every 14 days. In some embodiments, the antibody-drug conjugate is disitamab vedotin. In some embodiments, tucatinib is administered to the individual at a dose of between about 200 mg and about 300 mg. In some embodiments, tucatinib is administered to the individual at a dose of 200 mg, 250 mg, or 300 mg. In some embodiments, tucatinib is administered to the individual at a dose of 300 mg. In some embodiments, tucatinib is administered orally to the individual. In some embodiments, tucatinib is administered to the individual twice daily. In some embodiments, the method comprises intravenously administering disitamab vedotin to the individual at 1.0 mg/kg, 1.25 mg/kg, or 1.5 mg/kg with a maximum dose of 150 mg on Days 1, 15, and 29 of a 6-week cycle; and orally administering tucatinib to the individual at 300 mg twice daily starting at Day 8 of the 6-week cycle. In some embodiments, the method comprises intravenously administering disitamab vedotin to the individual at 1.0 mg/kg with a maximum dose of 150 mg on Days 1, 15, and 29 of a 6-week cycle; and orally administering tucatinib to the individual at 300 mg twice daily starting at Day 8 of the 6-week cycle. In some embodiments, the method comprises intravenously administering disitamab vedotin to the individual at 1.25 mg/kg with a maximum dose of 150 mg on Days 1, 15, and 29 of a 6-week cycle; and orally administering tucatinib to the individual at 300 mg twice daily starting at Day 8 of the 6-week cycle. In some embodiments, the method comprises intravenously administering disitamab vedotin to the individual at 1.5 mg/kg with a maximum dose of 150 mg on Days 1, 15, and 29 of a 6-week cycle; and orally administering tucatinib to the individual at 300 mg twice daily starting at Day 8 of the 6-week cycle.

In some embodiments according to any of the embodiments described herein, administration of tucatinib and the anti-HER2 antibody-drug conjugate results in increased internalization of the antibody-drug conjugate by cells of the cancer, as compared to administration of the antibody-drug conjugate in the absence of tucatinib. In some embodiments, administration of tucatinib and the antibody-drug conjugate results in increased expression of HER2 by cells of the cancer, as compared to administration of the antibody-drug conjugate in the absence of tucatinib. In some embodiments, administration of tucatinib and the antibody-drug conjugate results in increased total expression of HER2 by cells of the cancer, as compared to administration of the antibody-drug conjugate in the absence of tucatinib. In some embodiments, administration of tucatinib and the anti-HER2 antibody-drug conjugate results in increased surface expression of HER2 by cells of the cancer, as compared to administration of the antibody-drug conjugate in the absence of tucatinib. In some embodiments, administration of tucatinib and the antibody-drug conjugate results in a complete response (CR) or partial response (PR) in the individual.

In some embodiments according to any of the embodiments described herein, the individual is a human.

Further provided herein is a composition comprising an anti-HER2 antibody-drug conjugate for use in a method of treating or preventing progression of cancer in an individual, wherein the method comprises administering an effective amount of tucatinib and the anti-HER2 antibody-drug conjugate according to the method of any one of the above embodiments.

Further provided herein is a composition comprising an anti-HER2 antibody-drug conjugate and tucatinib for use in a method of treating or preventing progression of cancer in an individual, wherein the method comprises administering an effective amount of the composition according to the method of any one of the above embodiments.

Further provided herein is the use of a composition comprising an anti-HER2 antibody-drug conjugate in the manufacture of a medicament for treating or preventing progression of cancer in an individual, wherein the composition is to be administered with tucatinib according to the method of any one of the above embodiments.

Further provided herein is the use of a composition comprising an anti-HER2 antibody-drug conjugate and tucatinib in the manufacture of a medicament for treating or preventing progression of cancer in an individual, wherein the composition is to be administered according to the method of any one of the above embodiments.

Further provided herein is a kit or article or manufacture comprising tucatinib and an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

Further provided herein is a kit or article or manufacture comprising tucatinib and an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:11); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6). In some embodiments, the kit further comprises instructions for administering an effective amount of tucatinib and the antibody-drug conjugate to an individual in need thereof according to the method of any one of the above embodiments.

Further provided herein is a kit or article or manufacture comprising an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6); and instructions for administering an effective amount of the antibody-drug conjugate and tucatinib to an individual in need thereof according to the method of any one of the above embodiments.

Further provided herein is a kit or article or manufacture comprising: (a) an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:11); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6); and (b) instructions for administering an effective amount of the antibody-drug conjugate and tucatinib to an individual in need thereof according to the method of any one of the above embodiments.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1F show cytotoxicity curves (left) and drug combination computational analyses (right) of in vitro cytotoxicity assays in breast cancer cell lines treated with the indicated concentrations of disitamab vedotin (DV) and tucatinib (Tuc). In the depictions of computational analysis, blocks that tested as significant at a P-value of 0.01, adjusted for multiple testing by the total number of 3×3 blocks, in either direction (synergy or antagonism) were marked with dashed boxes. FIG. 1A shows results using the SK-BR-3 cell line (5×10⁵-1.5×10⁶ HER2 copies/cell). These results indicate synergy in SK-BR-3 cell killing with combined treatment of DV at 1.2e-1 ng/mL to 9.8e-1 ng/mL and tucatinib at 4.6e+0 nM to 4.1e+1 nM. FIG. 1B shows results using the BT-474 cell line (5×10⁵-1.5×10⁶ HER2 copies/cell). These results indicate synergy in BT-474 cell killing with combined treatment of DV at 1.2e-1 ng/mL to 6.4e+3 ng/mL and tucatinib at 4.6e+0 nM to 1.2e+2 nM. FIG. 1C shows results using the HCC-202 cell line (1×10⁵-5×10⁵ HER2 copies/cell). These results indicate synergy in HCC-202 cell killing with combined treatment of DV at 1.2e-1 ng/mL to 9.8e-1 ng/mL and tucatinib at 4.6e+0 nM to 4.1e+1 nM. FIG. 1D shows results using the EFM-192C cell line (1×10⁵-5×10⁵ HER2 copies/cell). These results indicate synergy in EFM-192C cell killing with combined treatment of DV at 1.2e-1 ng/mL to 9.8e-1 ng/mL and tucatinib at 1.2e+2 nM. FIG. 1E shows results using the BT-483 cell line (5×10⁴-1×10⁵ HER2 copies/cell). These results indicate synergy in BT-483 cell killing with combined treatment of DV at 1.2e-1 ng/mL to 2.9e-0 ng/mL and tucatinib at 4.6e+0 nM to 4.1e+1 nM. FIG. 1F shows results using the CAMA-1 cell line (5×10⁴-1×10⁵ HER2 copies/cell). There was no synergy found in CAMA-1 cell killing with the combined treatment of DV and tucatinib at the experimental concentrations.

FIGS. 2A-2C show cytotoxicity curves (left) and drug combination computational analyses (right) of in vitro cytotoxicity assays in gastric cancer cell lines treated with the indicated concentrations of disitamab vedotin (DV) and tucatinib (Tuc). In the depictions of computational analysis, blocks that tested as significant at a P-value of 0.01, adjusted for multiple testing by the total number of 3×3 blocks, in either direction (synergy or antagonism) were marked with dashed boxes. FIG. 2A shows results using the NCI-N87 cell line (5×10⁵-1.5×10⁶ HER2 copies/cell). These results indicate synergy in NCI-N87 cell killing with combined treatment of DV at 9.8e-1 ng/mL to 6.4e-3 ng/mL and tucatinib at 4.6e+0 nM to 1.2e+1 nM. FIG. 2B shows results using the OE19 cell line (1×10⁵-5×10⁵ HER2 copies/cell). These results indicate synergy in OE19 cell killing with combined treatment of DV at 1.2e-1 ng/mL to 2.9e-0 ng/mL and tucatinib at 4.6e+0 nM to 1.2e+2 nM. FIG. 2C shows results using the RERF-GC-1B cell line (5×10⁴-1×10⁵ HER2 copies/cell). These results indicate synergy in RERF-GC-1B cell killing with combined treatment of DV at 9.8e-1 ng/mL to 7.1e+2 ng/mL and tucatinib at 4.1e+1 nM to 1.2e+2 nM.

FIGS. 3A-3D show changes to HER2 cell surface protein levels upon treatment with 100 nM tucatinib in various cancer cell lines (black squares), as compared to untreated cells (gray circles). FIG. 3A shows results using the SK-BR-3 and BT-474 breast cancer cell lines (5×10⁵-1.5×10⁶ HER2 copies/cell). FIG. 3B shows results using the HCC-202 and EFM-192C breast cancer cell lines (1×10⁵-5×10⁵ HER2 copies/cell). FIG. 3C shows results using the BT-483 and CAMA-1 breast cancer cell lines (5×10⁴-1×10⁵ HER2 copies/cell). FIG. 3D shows results using the NCI-N87 (5×10⁵-1.5×10⁶ HER2 copies/cell), OE19 (1×10⁵-5×10⁵ HER2 copies/cell), and RERF-GC-1B (5×10⁴-1×10⁵ HER2 copies/cell) gastric cancer cell lines.

FIGS. 4A-4D show the results of internalization assays of disitamab in the presence (black circles) or absence (gray triangles) of tucatinib (100 nM) in various breast cancer cell lines, as compared to control IgG1-Fab (gray diamonds). FIG. 4A shows results using the SK-BR-3 cell line (5×10⁵-1.5×10⁶ HER2 copies/cell). FIG. 4B shows results using the BT-474 cell line (5×10⁵-1.5×10⁶ HER2 copies/cell). FIG. 4C shows results using the HCC-202 cell line (1×10⁵-5×10⁵ HER2 copies/cell). FIG. 4D shows results using the EFM-192C cell line (1×10⁵-5×10⁵ HER2 copies/cell).

FIGS. 5 & 6 show the results of internalization assays of disitamab in the presence (black circles) or absence (gray triangles) of tucatinib (Tuc, 100 nM) in various gastric cancer cell lines, as compared to control IgG1-Fab (gray diamonds). FIG. 5 shows results using the NCI-N87 cell line (5×10⁵-1.5×10⁶ HER2 copies/cell). FIG. 6 shows results using the OE19 cell line (1×10⁵-5×10⁵ HER2 copies/cell).

FIGS. 7A-7C show enhanced internalization of disitamab compared to trastuzumab in breast cancer cells with variable HER2 expression. FIG. 7A shows results using the SK-BR-3 and BT-474 cell lines (5×10⁵-1.5×10⁶ HER2 copies/cell). FIG. 7B shows results using the EFM-192C and HCC-202 cell lines (1×10⁵-5×10⁵ HER2 copies/cell). FIG. 7C shows results using the BT-483 and CAMA-1 cell lines (5×10⁴-1×10⁵ HER2 copies/cell). FIGS. 7D & 7E show internalization of disitamab vedotin (DV) compared to trastuzumab emtansine (T-DM1) and trastuzumab deruxtecan (T-DXd) in breast cancer cells with variable HER2 expression. FIG. 7D shows results using the SK-BR-3 and HCC-202 cell lines. FIG. 7E shows results using the BT-483 cell line.

FIG. 8A shows enhanced internalization of disitamab compared to trastuzumab in gastric cancer cells NCI-N87, OE19, and RERF-GC-1B with variable HER2 expression. FIG. 8B shows in vitro cytotoxicity of disitamab vedotin (DV), trastuzumab emtansine (T-DM1), and trastuzumab deruxtecan (T-DXd) against a panel of breast cancer cell lines (as indicated) spanning a range of HER2 expression levels. IC50 values (concentration required for 50% inhibition) for each ADC against each cell line are provided, along with HER2 receptor copy number of each cell line, as calculated by quantitative flow cytometry for surface expression.

FIG. 9 shows the schema for a Phase 1b/2 open-label study of disitamab vedotin monotherapy or in combination with other anti-cancer therapies (e.g., tucatinib) in solid tumors. 2L=second-line; 3L=third-line; BID=twice a day (bis in die); DLT=dose limiting toxicity; DV=disitamab vedotin; GC/GEJC=gastric cancer and gastroesophageal junction adenocarcinoma; HER2=human epidermal growth factor receptor 2; IHC=immunohistochemistry; ISH=in situ hybridization; mets=metastases; Q2W=every 2 weeks. ^{a}Staggered dosing Cycle 1, tucatinib dosing will start on Day 8. ^{b}Example 4 describes visceral metastases.

FIG. 10A shows the anti-tumor effects of a single dose of DV as a monotherapy in HER2-low breast cancer patient-derived xenograft (PDX) models. Doses used were 0.3, 1, 2, or 5 mg/kg, as indicated. PDX models were CTG-0670 (IHC 2+), CTG-0012 (IHC 1+), and CTG-1520 (IHC 0), as indicated.

FIGS. 10B-10D show the effects of combining DV and tucatinib vs. either single agent in HER2-low breast cancer patient-derived xenograft (PDX) models: CTG-0670 (IHC 2+; FIG. 10B), CTG-0012 (IHC 1+; FIG. 10C), and CTG-1520 (IHC 0; FIG. 10D). Representative IHC images for each model are also shown.

FIGS. 11A and 11B show the results of in vitro cytotoxicity assays in 2D breast cancer cell lines (FIG. 11A, as indicated) and in patient-derived organoids (PDOs; FIG. 11B, as indicated) comparing DV and trastuzumab deruxtecan (T-DXd). HER2 expression level for each cell line/PDO is indicated and was measured by quantitative flow cytometry. (T-DXd data from day 12; DV data from day 8. Data shown as mean±SEM).

FIGS. 12A-12F show the effects of combining DV and tucatinib vs. DV as a single agent in gastric cancer patient-derived xenograft (PDX) models with varying HER2 expression levels: GA13745 (IHC 1+; FIGS. 12A, 12B), GA6821 (IHC 1+; FIGS. 12C, 12D), CTG-0148 (IHC 1+; FIGS. 12E, 12F), CTG-3036 (IHC 2+; FIGS. 12G, 12H), CTG-3033 (IHC 3+; FIGS. 12I, 12J), and GA3102 (IHC 3+; FIGS. 12K, 12L). Treatment with T-DXd, a HER2-directed ADC, was also included in the study. Tumor volume data shown as mean±SEM. For each model, tumor growth was evaluated using a normalized area under the curve (AUC) metric (see, Guo, S. et al. (2019) BMC Cancer 19, 718) (FIGS. 12A, 12C, 12E, 12G, 12I, 12K). AUC was calculated for each model for the duration of the experiment, then compared by one-way ANOVA followed by Tukey's post-hoc test. The box-and-whisker plots represent the median and range for each treatment group (FIGS. 12B, 12D, 12F, 12H, 12J, 12L). Asterisks indicate significance of selected comparisons from Tukey's post-hoc test: ****P<0.0001; ***P<0.001; **P<0.01; ns, not significant.

### DETAILED DESCRIPTION

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%” When “about” is applied to the first value of a set of values, it applies to all values in that set. Thus, “about 7, 9, or 11 mg/kg” is equivalent to “about 7, about 9, or about 11 mg/kg.”

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

As used herein, the term “administering” when referring to two or more components (e.g., tucatinib and an anti-HER2 antibody-drug conjugate) includes sequential or simultaneous administration of tucatinib and the anti-HER2 antibody-drug conjugate. In some instances, the co-administered compounds are administered via different routes. For example, one or two compounds can be administered orally, and the other compound(s) can be administered, e.g, sequentially or simultaneously, via intravenous, intramuscular, subcutaneous, or intraperitoneal injection. The simultaneously or sequentially administered compounds or compositions can be administered such that the anti-HER2 antibody-drug conjugate and tucatinib are simultaneously present in a subject or in a cell at an effective concentration.

“Simultaneous administration,” as used herein, means that the two or more therapies (e.g., in a combination therapy) are administered with a time separation of no more than about 15 minutes, such as no more than about any of 10, 5, or 1 minutes. When the two or more therapies are administered simultaneously, the two or more therapies can be contained in the same composition (e.g., a composition comprising both a first and second therapy) or in separate compositions (e.g., a first therapy in one composition and a second therapy is contained in another composition).

As used herein, the term “sequential administration” means that the two or more therapies (e.g., in a combination therapy) are administered with a time separation of more than about 15 minutes, such as more than about any of 20, 30, 40, 50, 60, or more minutes. Any of the two or more therapies may be administered first. The two or more therapies are contained in separate compositions, which may be contained in the same or different packages or kits.

As used herein, the term “concurrent administration” means that the administration of two or more therapies (e.g., in a combination therapy) overlap with each other. For example, the two or more therapies may be administered in the same day, or with a time separation of within one day, within two days, within three days, within four days, within five days, within six days, within seven days, within ten days, within fourteen days, or within twenty-one days.

A “cancer” refers to a broad group of various diseases characterized by the uncontrolled growth of abnormal cells in the body. A “cancer” or “cancer tissue” can include a tumor.

The term “metastasis” is an art known term that refers to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, lung cancer (e.g non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP 001005862, NP_001289936, NP_001289937, NP_001289938, and NP_004448. Non-limiting examples of HER2 peptide sequences are set forth in GenBank reference numbers NP OO 1005862, NP 001276865, NP_001276866, NP_001276867, and NP_004439.

The term “anti-HER2 antibody-drug conjugate” refers to an anti-HER2 antibody conjugated to a therapeutic agent {i.e., a drug or cytotoxic molecule) optionally via a linker.

An “anti-HER2 antibody”, as used herein, refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present invention include trastuzumab, pertuzumab, margetuximab, and combinations thereof.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer. As used herein, the terms “treatment” and “treating” when referring, e.g., to the treatment of a cancer, are not intended to be absolute terms. For example, “treatment of cancer” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQC30), decreasing the dose of other medications required to treat a disease, delaying the progression of a disease, and/or prolonging survival of subjects having a disease. [110] The term “prophylactic” or “prophylactically” refers to any type of intervention or process performed on, or the administration of an active agent to, the subject with the objective of protecting or preventing a disease or condition from developing or at least not developing fully (e.g., to reduce the symptoms or severity of the disease or condition) such as in the development of a side effect (e.g., diarrhea).

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

The phrase “pharmaceutically acceptable salt” as used herein, refers to pharmaceutically acceptable organic or inorganic salts of a compound of the disclosure. Exemplary salts include, but are not limited, to sulfate, citrate, acetate, oxalate, chloride, bromide, iodide, nitrate, bi sulfate, phosphate, acid phosphate, isonicotinate, lactate, salicylate, acid citrate, tartrate, oleate, tannate, pantothenate, bitartrate, ascorbate, succinate, maleate, gen isinate, fumarate, gluconate, g!ucuronate, saccbarate, formate, benzoate, glutamate, methanesulfonate “mesylate”, ethanesulfonate, benzenesulfonate, p-toluenesulfonate, pamoate (i.e., 4,4′-methyl ene-bis-(2-hydroxy-3-napbthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

### II. Methods of Treating Cancer

Certain aspects of the present disclosure relate to methods for treating or preventing progression of cancer in an individual, comprising administering to the individual an effective amount of tucatinib and an anti-human epidermal growth factor receptor 2 (HER2) antibody-drug conjugate. In some embodiments, the anti-HER2 antibody-drug conjugate comprises an anti-HER2 antibody and a cytotoxic molecule (e.g., wherein one or more cytotoxic molecule(s) is/are linked the anti-HER2 antibody directly or via linker).

### Antibody-Drug Conjugates

In some embodiments, the anti-HER2 antibody comprises a heavy chain comprising a heavy chain variable (VH) domain and a light chain comprising a light chain variable (VL) domain. In some embodiments, the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3) or ARNYLFDHW (SEQ ID NO:11); and/or the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6). In some embodiments, the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); and the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6). In some embodiments, the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO: 11); and the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

In some embodiments, the anti-HER2 antibody comprises a VH domain that comprises the amino acid sequence of SEQ ID NO:7 and/or a VL domain that comprises the amino acid sequence of SEQ ID NO:8. In some embodiments, the anti-HER2 antibody comprises a VH domain that comprises the amino acid sequence of SEQ ID NO:7 and a VL domain that comprises the amino acid sequence of SEQ ID NO:8.

In some embodiments, the anti-HER2 antibody comprises a heavy chain that comprises the amino acid sequence of SEQ ID NO:9 and/or a light chain that comprises the amino acid sequence of SEQ ID NO:10. In some embodiments, the anti-HER2 antibody comprises a heavy chain that comprises the amino acid sequence of SEQ ID NO:9 and a light chain that comprises the amino acid sequence of SEQ ID NO:10.

In some embodiments, the anti-HER2 antibody comprises one, two, three, four, five, or six CDR sequences shown in Table A below. In some embodiments, the anti-HER2 antibody comprises one, two, or all three CDR sequences from a VH domain or heavy chain sequence shown in Table A below and one, two, or all three CDR sequences from a VL domain or light chain sequence shown in Table A below. In some embodiments, the anti-HER2 antibody comprises a VH domain sequence and/or VL domain sequence shown in Table A below. In some embodiments, the anti-HER2 antibody comprises a heavy chain and/or light chain sequence shown in Table A below.

TABLE A
Anti-HER2 antibody sequences

| | | SEQ |
|---|---|---|
| Description | Amino acid sequence | ID NO |
| Disitamab | DYYIH | 1 |
| CDR-H1 | | |
| Disitamab | RVNPDHGDSYYNQKFKD | 2 |
| CDR-H2 | | |
| Disitamab | NYLFDH | 3 |
| CDR-H3 | | |
| Disitamab | KASQDVGTAVA | 4 |
| CDR-L1 | | |
| Disitamab | WASIRHT | 5 |
| CDR-L2 | | |
| Disitamab | HQFATYT | 6 |
| CDR-L3 | | |
| Disitamab | EVQLVQSGAEVKKPGATVKISCKVSGYTFTDYYIHWV | 7 |
| VH domain | QQAPGKGLEWMGRVNPDHGDSYYNQKFKDKATITAD | |
| | KSTDTAYMELSSLRSEDTAVYFCARNYLFDHWGQGTL | |
| | VTVSS | |
| Disitamab | DIQMTQSPSSVSASVGDRVTITCKASQDVGTAVAWYQ | 8 |
| VL domain | QKPGKAPKLLIYWASIRHTGVPSRFSGSGSGTDFTLTIS | |
| | SLQPEDFATYYCHQFATYTFGGGTKVEIK | |
| Disitamab | EVQLVQSGAEVKKPGATVKISCKVSGYTFTDYYIHWV | 9 |
| heavy | QQAPGKGLEWMGRVNPDHGDSYYNQKFKDKATITAD | |
| chain | KSTDTAYMELSSLRSEDTAVYFCARNYLFDHWGQGTL | |
| | VTVSSASTKGPSVFPLAPSSKSTSGGTAALGCLVKDYF | |
| | PEPVTVSWNSGALTSGVHTFPAVLQSSGLYSLSSVVTV | |
| | PSSSLGTQTYICNVNHKPSNTKVDKKVEPKSCDKTHTC | |
| | PPCPAPELLGGPSVFLFPPKPKDTLMISRTPEVTCVVVD | |
| | VSHEDPEVKFNWYVDGVEVHNAKTKPREEQYNSTYR | |
| | VVSVLTVLHQDWLNGKEYKCKVSNKALPAPIEKTISK | |
| | AKGQPREPQVYTLPPSREEMTKNQVSLTCLVKGFYPSD | |
| | IAVEWESNGQPENNYKTTPPVLDSDGSFFLYSKLTVDK | |
| | SRWQQGNVFSCSVMHEALHNHYTQKSLSLSPG | |
| Disitamab | DIQMTQSPSSVSASVGDRVTITCKASQDVGTAVAWYQ | 10 |
| light chain | QKPGKAPKLLIYWASIRHTGVPSRFSGSGSGTDFTLTIS | |
| | SLQPEDFATYYCHQFATYTFGGGTKVEIKRTVAAPSVF | |
| | IFPPSDEQLKSGTASVVCLLNNFYPREAKVQWKVDNA | |
| | LQSGNSQESVTEQDSKDSTYSLSSTLTLSKADYEKHKV | |
| | YACEVTHQGLSSPVTKSFNRGEC | |
| Disitamab | ARNYLFDHW | 11 |
| CDR-H3 | | |
| (long) | | |

In some embodiments, the anti-HER2 antibody is disitamab (RC48). See, e.g., U.S. Pat. No. 10,087,260.

In some embodiments, the cytotoxic molecule of an antibody-drug conjugate of the present disclosure comprises a tubulin inhibitor or DNA damaging agent.

In some embodiments, the tubulin inhibitor comprises a dolastatin or derivative thereof, auristatin or derivative thereof, or maytansinoid or derivative thereof. In some embodiments, the tubulin inhibitor comprises monomethyl auristatin E (MMAE), monomethyl auristatin F (MMAF), or auristatin F (AF). In some embodiments, the tubulin inhibitor comprises mertansine (DM1), maytansine (DM3), or ravtansine (DM4). Structures for MMAE and MMAF are provided below. Additional descriptions and examples of tubulin inhibitors may be found, e.g., in Chen, H. et al. (2017) Molecules 22(8):1281.

In some embodiments, the DNA damaging agent comprises a calicheamicin, duocarmycin, pyrrolobenzodiazepine (PBD), or SN-38.

In some embodiments, the cytotoxic molecule comprises an amanitin, anthracycline, baccatin, camptothecin, cemadotin, colchicine, colcimid, combretastatin, cryptophycin, dicodermolide, docetaxel, doxorubicin, echinomycin, eleutherobin, epothilone, estramustine, lexitropsin, maytansine, methotrexate, netropsin, puromycin, rhizoxins, taxane, tubulysin, or vinca alkaloid.

In some embodiments, the antibody-drug conjugate is represented by formula Ab-(L-U)n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody. For example, in some embodiments, the antibody-drug conjugate used is named RC48-mc-vc-pAB-MMAE, which conforms to the structure of the general formula Ab-(L-U)n, in which RC48 (a humanized anti-HER2 monoclonal antibody) is coupled to MMAE through the linker mc-vc-pAB, and the number of coupling ranges from 1 to 8, including 1, 2, 3, 4, 5, 6, 7, 8 or a combination of antibody-drug conjugates with varying MMAE coupling numbers ranging from 1 to 8. In some embodiments, the number of cytotoxic molecules bound to the antibody is given as an average number of cytotoxic molecules bound to the antibody, e.g., within a given sample, population, or composition. In some embodiments, an average of 4 MMAE molecules are conjugated to the antibody, e.g., via a linker such as mc-vc-pAB.

In some embodiments, the linker is attached to the anti-HER2 antibody via a thiol or amino moiety. In some embodiments, the cytotoxic molecule is conjugated to the antibody through site-directed or undirected conjugation.

In some embodiments, the linker is selected from the group consisting of maleimidocaproyl valine citrulline p-amino-benzyloxy (mc-vc-pAB), maleimidocaproyl (mc), triglycyl peptide linker, 3-maleimido-propionic acid, Mal-di-EG-OPFP (perfluorophenyl 3-(2-(2-(3-(2,5-dioxo-2,5-dihydro-1H-pyrrol-1-yl)propanamido)ethoxy)ethoxy)propanoate), Mal-di-EG-OSu (2,5-dioxopyrrolidin-1-yl 3-(2-(2-(2,5-dioxo-2,5-dihydro-1H-pyrrol-1-yl)ethoxy)ethoxy)propanoate), Mal-Tri-EG-OSu (2,5-dioxopyrrolidin-1-yl 3-(2-(2-(2-(2,5-dioxo-2,5-dihydro-1H-pyrrol-1-yl)ethoxy)ethoxy)ethoxy)ethoxypropanoate), Mal-Tetra-EG-OSu (2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate), Br-di-EG-OSu (2,5-dioxopyrrolidin-1-yl 3 (2-(2-(2-bromoacetamido)ethoxy)ethoxy)propanoate), Py-ds-prp-OSu (2-5-dioxopyrrolidin-1-yl 3-(pyridine-2-yldisulfanyl)propanoate), Py-ds-Prp-OPEP (perfluorophenyl 3-(pyridine-2-yldisulfanyl)propanoate), Py-ds-dmBut-OSu (2,5-dioxopyrrolidin-1-yl 4-methyl-4-(pyridine-2-yldisulfanyl)pentanoate, Py-ds-dmBut-OPF (perfluorophenyl 4-methyl-4-(pyridine-2-yldisulfanyl)pentanoate), SMCC (N-succinimidyl 4-(maleimidomethyl) cyclohexanecarboxylate), MBS (3-maleimidobenzoic acid N-hydroxysuccinimide ester), SATA (S—(N-succinimidyl)thioacetate), SPDP ((N-succinimidyl 3-(2-pyridyldithio)propionate), and SMPT ((N-succinimidyloxy carbonyl)-1-methyl-1-(2-pyridyldithio)toluene).

In some embodiments, the linker is a linker described in Table B below.

TABLE B
Exemplary linkers for antibody-drug conjugates.

| Abbreviation | Full name | Feature |
|---|---|---|
| mc | Maleimidocaproyl | Non- |
| | | cleavable |
| mc-vc-pAB | Maleimidocaproyl valine citrulline p-amino-benzyloxy | Cleavable |
| 3-MPA | 3-maleimido-propionic acid | Non- |
| | | cleavable |
| Mal-di-EG- | Perfluorophenyl | Non- |
| OPFP | 3-(2-(2-(3-(2,5-dioxo-2,5-dihydro-1H-pyrrol-1- | cleavable |
| | yl)propanamido)ethoxy) ethoxy)propanoate | |
| Mal-di-EG-OSu | 2,5-dioxopyrrolidin-1-yl | Non- |
| | 3-(2-(2-(2,5-dioxo-2,5-dihydro-1H-pyrrol-1- | cleavable |
| | yl)ethoxy)ethoxy) propanoate | |
| Mal-Tri-EG- | 2,5-dioxopyrrolidin-1-yl | Non- |
| OSu | 3-(2-(2-(2-(2,5-dioxo-2,5-dihydro-1H-pyrrol-1- | cleavable |
| | yl)ethoxy)ethoxy)ethoxy) propanoate | |
| Mal-Tetra-EG- | 2,5-dioxopyrrolidin-1-yl | Non- |
| OSu | 1-(2,5-dioxo-2,5-dihydro-1H-pyrrol-1-yl)-3-oxo- | cleavable |
| | 7,10,13,16-tetraoxa-4- | |
| | azanonadecan-19-oate | |
| Br-di-EG-OSu | 2,5-dioxopyrrolidin-1-yl | Non- |
| | 3-(2-(2-(2-bromoacetamido)ethoxy)ethoxy)propanoate | cleavable |
| Py-ds-prp-OSu | 2,5-dioxopyrrolidin-1-yl 3-(pyridin-2- | Reducible |
| | yldisulfanyl)propanoate | |
| Py-ds-Prp-OPFP | Perfluorophenyl 3-(pyridin-2-yldisulfanyl)propanoate | Reducible |
| Py-ds-dmBut- | 2,5-dioxopyrrolidin-1-yl 4-methyl-4-(pyridin-2- | Reducible |
| OSu | yldisulfanyl) | |
| | pentanoate | |
| Py-ds-dmBut- | Perfluorophenyl 4-methyl-4-(pyridin-2- | Reducible |
| OPF | yldisulfanyl)pentanoate | |
| SMCC | N-succinimidyl 4-(maleimidomethyl) | Non- |
| | cyclohexanecarboxylate | cleavable |
| MBS | 3-maleimidobenzoic acid N-hydroxysuccinimide ester | Non- |
| | | cleavable |
| SATA | S-(N-succinimidyl) thioacetate | Non- |
| | | cleavable |
| SPDP | N-succinimidyl 3-(2-pyridyldithio)propionate | Reducible |
| SMPT | (N-succinimidyl carbonyl)-1-methyl-1-(2- | Reducible |
| | pyridyldithio) toluene | |

In some embodiments, the anti-HER2 antibody-drug conjugate is disitamab vedotin (DV; RC48-ADC). Disitamab vedotin (DV, RC48-ADC) is an antibody-drug conjugate (ADC) that targets cancers expressing HER2, an oncogenic growth factor receptor which promotes cell proliferation and survival. DV consists of an anti-HER2 monoclonal antibody disitamab (RC48) conjugated with 4 molecules of the tubulin-disrupting anti-mitotic agent monomethyl auristatin E (MMAE) via a cleavable peptide linker. See, e.g., U.S. Pat. No. 10,087,260 and U.S. PG Pub Nos. 2020/0289663 and 2021/0154314. DV has multimodal antitumor mechanisms of action that include direct cytotoxicity of HER2-expressing cancer cells and bystander effect based-cytotoxicity of neighboring cells, both of which are mediated by the intracellular release of MMAE within the targeted cell. Released MMAE can induce immunogenic cell death (ICD), which promotes immune cell recruitment to the tumor. In addition, DV stimulates Fc-gamma receptor mediated antibody-dependent cellular cytotoxicity (ADCC), which can lead to target cell death. DV also inhibits HER2-activated downstream signaling pathways, further blocking cellular growth and proliferation.

### Tucatinib

Tucatinib, also known as ONT-380 and ARRY-380, refers to the small molecule tyrosine kinase inhibitor that suppresses or blocks HER2 activation. The structure of tucatinib is shown below. In some instances, tucatinib can be in the form of a pharmaceutically acceptable salt.

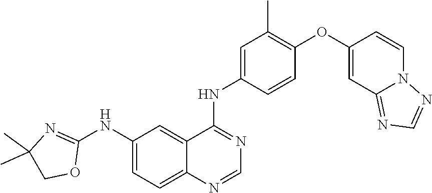 [SMILES: Cc1cc(Nc2ncnc3ccc(NC4=NC(C)(C)CO4)cc23)ccc1Oc1ccn2ncnc2c1]

In some embodiments, tucatinib is administered in a pharmaceutical composition comprising tucatinib and one or more pharmaceutically acceptable carriers. Additional descriptions of tucatinib and formulations thereof can be found, e.g., in International Pub. Nos. WO2021/097220 and WO2022/067347. The pharmaceutical compositions are typically administered orally. The pharmaceutical compositions are typically administered as a tablet, caplet, hard or soft gelatin capsule, pill, granules or a suspension. Additional examples of pharmaceutical compositions of tucatinib and methods of preparation thereof are described in U.S. Pat. No. 9,457,093.

In certain embodiments, the pharmaceutical composition contains between about 25 and about 400 mg of tucatinib. In certain embodiments, the pharmaceutical composition contains between 25 and 400 mg of tucatinib.

In certain embodiments, the pharmaceutical composition contains between about 25 and about 100 mg (e.g., about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg) of tucatinib. In certain embodiments, the pharmaceutical composition contains between 25 and 100 mg (e.g., 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg) of tucatinib. In certain embodiments, the pharmaceutical composition contains between about 25 and about 75 mg of tucatinib. In certain embodiments, the pharmaceutical composition contains between 25 and 75 mg of tucatinib. In certain embodiments, the pharmaceutical composition contains about 50 mg of tucatinib. In certain particular embodiments, the pharmaceutical composition contains 50 mg of tucatinib. In certain of the foregoing embodiments, the pharmaceutical composition is formulated as a tablet. As a non-limiting example, the pharmaceutical composition is formulated as a tablet and contains 50 mg of tucatinib.

In some embodiments, the pharmaceutical composition contains between about 100 and about 300 mg (e.g., about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg) of tucatinib. In certain embodiments, the pharmaceutical composition contains between 100 and 300 mg (e.g, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg) of tucatinib. In certain embodiments, the pharmaceutical composition contains between about 100 and about 200 mg of tucatinib. In certain embodiments, the pharmaceutical composition contains between 100 and 200 mg of tucatinib. In certain embodiments, the pharmaceutical composition contains between about 125 and about 175 mg of tucatinib. In certain embodiments, the pharmaceutical composition contains between 125 and 175 mg of tucatinib. In certain embodiments, the pharmaceutical composition contains about 150 mg of tucatinib. In certain particular embodiments, the pharmaceutical composition contains 150 mg of tucatinib. In certain of the foregoing embodiments, the pharmaceutical composition is formulated as a tablet. As a non-limiting example, the pharmaceutical composition is formulated as a tablet and contains 150 mg of tucatinib.

In some embodiments, tucatinib is present in a pharmaceutical composition comprising:

| Function | Ingredient | % of Blend |
|---|---|---|
| API | Solid dispersion of tucatinib | about 50 |
| Disintegrant | Crospovidone - | about 6 |
| | Polyplasdone ® | |
| Osmogen | NaCl | about 5 |
| Osmogen | KCl | about 5 |
| Glidant | Colloidal Silicon Dioxide | about 0.5 |
| Lubricant | Magnesium Stearate | about 0.25 |

Extragranular

| | | |
|---|---|---|
| Binder/Diluent | Microcrystalline | about 19.25 |
| | cellulose - Avicel ® | |
| Osmogen | NaCl | about 4.625 |
| Osmogen | KCl | about 4.625 |
| Disintegrant | Polyplasdone | about 4 |
| Glidant | Colloidal Silicon Dioxide | about 0.5 |
| Lubricant | Magnesium Stearate | about 0.25 |

In some embodiments, tucatinib is present in a pharmaceutical composition comprising:

| Function | Ingredient | % of Blend |
|---|---|---|
| API | Solid dispersion of tucatinib | about 50 |
| Disintegrant | Crospovidone - | about 6 |
| | Polyplasdone ® | |
| Disintegrant | NaHCO₃ | about 3 |
| Osmogen | NaCl | about 5 |
| Osmogen | KCl | about 5 |
| Glidant | Colloidal Silicon Dioxide | about 0.5 |
| Lubricant | Magnesium Stearate | about 0.25 |

Extragranular

| | | |
|---|---|---|
| Binder/Diluent | Microcrystalline | about 16.25 |
| | cellulose - Avicel ® | |
| Osmogen | NaCl | about 4.625 |
| Osmogen | KCl | about 4.625 |
| Disintegrant | Polyplasdone | about 4 |
| Glidant | Colloidal Silicon Dioxide | about 0.5 |
| Lubricant | Magnesium Stearate | about 0.25 |

In some embodiments, tucatinib is present in a pharmaceutical composition PP-7T comprising:

| Function | Ingredient | % of Blend |
|---|---|---|
| API | Solid dispersion of tucatinib | about 50 |
| Disintegrant | Crospovidone - | about 6 |
| | Polyplasdone ® | |
| Osmogen | NaCl | about 10.625 |
| Osmogen | KCl | about 10.625 |
| Filler | Lactose | about 21.25 |
| Glidant | Colloidal Silicon Dioxide | about 0.5 |
| Lubricant | Magnesium Stearate | aabout 0.25 |

Extragranular

| | | |
|---|---|---|
| Glidant | Colloidal Silicon Dioxide | about 0.5 |
| Lubricant | Magnesium Stearate | about 0.25 |

In some embodiments, tucatinib is present in a pharmaceutical composition PP-7T comprising:

| Function | Ingredient | % of Blend |
|---|---|---|
| API | Solid dispersion of tucatinib | 50 |
| Disintegrant | Crospovidone - | 6 |
| | Polyplasdone ® | |
| Osmogen | NaCl | 5 |
| Osmogen | KCl | 5 |
| Glidant | Colloidal Silicon Dioxide | 0.5 |
| Lubricant | Magnesium Stearate | 0.25 |

Extragranular

| | | |
|---|---|---|
| Binder/Diluent | Microcrystalline | 19.25 |
| | cellulose - Avicel ® | |
| Osmogen | NaCl | 4.625 |
| Osmogen | KCl | 4.625 |
| Disintegrant | Polyplasdone | 4 |
| Glidant | Colloidal Silicon Dioxide | 0.5 |
| Lubricant | Magnesium Stearate | 0.25 |

In some embodiments, tucatinib is present in a pharmaceutical composition comprising:

| Function | Ingredient | % of Blend |
|---|---|---|
| API | Solid dispersion of tucatinib | 50 |
| Disintegrant | Crospovidone - | 6 |
| | Polyplasdone ® | |
| Disintegrant | NaHCO₃ | 3 |
| Osmogen | NaCl | 5 |
| Osmogen | KCl | 5 |
| Glidant | Colloidal Silicon Dioxide | 0.5 |
| Lubricant | Magnesium Stearate | 0.25 |

Extragranular

| | | |
|---|---|---|
| Binder/Diluent | Microcrystalline | 16.25 |
| | cellulose - Avicel ® | |
| Osmogen | NaCl | 4.625 |
| Osmogen | KCl | 4.625 |
| Disintegrant | Polyplasdone | 4 |
| Glidant | Colloidal Silicon Dioxide | 0.5 |
| Lubricant | Magnesium Stearate | 0.25 |

In some embodiments, tucatinib is present in a pharmaceutical composition comprising:

| Function | Ingredient | % of Blend |
|---|---|---|
| API | Solid dispersion of tucatinib | 50 |
| Disintegrant | Crospovidone - | 6 |
| | Polyplasdone ® | |
| Osmogen | NaCl | 10.625 |
| Osmogen | KCl | 10.625 |
| Filler | Lactose | 21.25 |
| Glidant | Colloidal Silicon Dioxide | 0.5 |
| Lubricant | Magnesium Stearate | 0.25 |

Extragranular

| | | |
|---|---|---|
| Glidant | Colloidal Silicon Dioxide | 0.5 |
| Lubricant | Magnesium Stearate | 0.25 |

### Cancers

In some embodiments, the cancer is breast cancer, gastric cancer, or gastroesophageal junction cancer (GEJC). In some embodiments, the cancer is a locally advanced or metastatic cancer. In some embodiments, the cancer is gastric adenocarcinoma or gastroesophageal junction adenocarcinoma. In some embodiments, the cancer is locally advanced or metastatic gastric cancer or GEJC (LA/mGC/GEJC). In some embodiments, the cancer is locally advanced or metastatic breast cancer (LA/mBC).

In some embodiments, cells of the cancer express HER2. In some embodiments, cells of the cancer exhibit HER2 gene amplification. In some embodiments, cells of the cancer overexpress HTER2, e.g., on their cell surface. Overexpression and/or amplification of HER2 is seen in many malignancies including breast, gastric, ovarian, pancreatic, colorectal, and endometrial cancer (Neve, R. M. et al. (2001) Ann Oncol 12(Suppl1):S9-S13; Menard, S. et al. (2003) Oncogene 22(42):6570-6578; Moasser, M. M. (2007) Oncogene 26(45):6469-6487; Iqbal, N. et al. (2014) Mol Biol Int 2014:852748).

In some embodiments, a sample obtained from the individual comprises cancer cells that express HTER2 on their cell surface, as measured by an immunohistochemistry (IHC) assay. IHC assays for HTER2 involve semi-quantitative measurements of HER2 cellular expression/overexpression. These are typically done by staining a sample including cancer cells using a primary anti-HER2 antibody, followed by visualization using a labeled secondary antibody and subsequent analysis of HER2 expression, e.g., membrane expression on the surface. Variables factoring into the IHC score (given on a scale from 0 to 3+) can include intensity of staining, location and/or completeness of staining (e.g., surface/membrane expression), and proportion of tumor cells exhibiting staining. Methods and criteria for determining HER2 status by IHC are known in the art and can be found, e.g., in Wolff, A. C. et al. (2013) J Clin Oncol 31(31):3997-4013 and Wolff, A. C. et al. (2018) J Clin Oncol 36(20):2105-2122. See also the HercepTest™ semi-quantitative RIC test kit.

In some embodiments, a sample obtained from the individual comprises cancer cells that exhibit HER2 gene amplification, as measured by an in situ hybridization (ISH) assay (ISH-positive). In some embodiments, a sample obtained from the individual comprises cancer cells that do not exhibit HER2 gene amplification, as measured by an in situ hybridization (ISH) assay (ISH-negative). ISH assays for HER2 gene amplification typically involve measurement of level of hybridization to a HER2-specific probe using microscopy. In some embodiments, cells are stained with dual probes: a HER2-specific probe, and a control probe (hybridizing to, e.g., chromosome 17 or CEP17), such that the ratio of HER2:control signal is indicative of HER2 copy number and/or amplification. Variables factoring into the ISH status can include number of signal copies of HER2, ratio of HER2:control copy number, and formation of HER2 clusters. Methods and criteria for determining HER2 amplification status by ISH are known in the art and can be found, e.g., in Wolff, A. C. et al. (2013) J Clin Oncol 31(31):3997-4013 and Wolff, A. C. et al. (2018) J Clin Oncol 36(20):2105-2122.

In some embodiments, the sample is a biopsy sample, e.g., from a core needle biopsy. In some embodiments, the sample is from an incisional or excisional surgical procedure. In some embodiments, the sample is a formalin-fixed paraffin embedded (FFPE) tissue block or sample, e.g., with corresponding H&E stain. In some embodiments, the sample comprises unstained slides sectioned from a tissue block, e.g., FFPE tissue block. In some embodiments, the sample is from a primary tumor or metastasis, e.g., from the chest wall, regional lymph node, or a distant organ. In some embodiments, the same sample is used for IHC and ISH assays. In some embodiments, different samples from the same individual are used for IHC and ISH assays.

Depending on tumor biology defined by immunohistochemistry (IHC) and in-situ hybridization (ISH), breast cancer is categorized into various subtypes based on level of HER2 expression. In clinical practice, breast cancers with high HER2 expression, defined by a score of IHC 3+ or IHC 2+ with HER2 gene amplification (ISH+), are considered to be HER2-positive (HER2+) (Wolff, A. C. et al. (2013) J Clin Oncol 31(31):3997-4013; Wolff, A. C. et al. (2018) J Cin Oncol 36(20):2105-2122; Wolff, A. C. et al. (2018) Arch Pathol Lab Med. 142(11):1364-1382). Breast cancers with limited to no HER2 expression, defined by a score of either IHC 0+, IHC 1+, or IHC 2+ without HER2 gene amplification (ISH-negative), are categorized as HER2-negative (Wolff, A. C. et al. (2013) J Clin Oncol 31(31):3997-4013; Wolff, A. C. et al. (2018) J Clin Oncol 36(20):2105-2122; Wolff, A. C. et al. (2018) Arch Pathol Lab Med. 142(11):1364-1382). Approximately 10%-20% of breast tumors are thought to be HER2+ and 80%-90% are thought to be HER2-negative (Cronin, K. A. et al. (2010) Cancer Invest 28(9):963-968; Tarantino, P. et al. (2020) J Clin Oncol 38(17):1951-1962; Schettini, F. et al. (2021) NPJ Breast Cancer 7(1):1).

In some embodiments, the cancer is a HER2-low cancer. In some embodiments, a sample obtained from the individual (e.g., from the cancer of the individual) comprises cancer cells that express HER2 on their cell surface at a level of IHC1+, as measured by IHC assay (IHC1+). In some embodiments, a sample obtained from the individual (e.g., from the cancer of the individual) comprises cancer cells that express HER2 on their cell surface at a level of IHC2+, as measured by IHC assay; and a sample obtained from the individual comprises cancer cells that do not exhibit HER2 gene amplification, as measured by ISH assay (IHC2+/ISH-negative). In some embodiments, a sample obtained from the individual (e.g., from the cancer of the individual) is IHC1+. In some embodiments, a sample obtained from the individual (e.g., from the cancer of the individual) is IHC2+/ISH-negative.

In some embodiments, the cancer is a HER2-positive cancer. In some embodiments, a sample obtained from the individual (e.g., from the cancer of the individual) comprises cancer cells that express HER2 on their cell surface at a level of IHC3+, as measured by IHC assay (IHC3+). In some embodiments, a sample obtained from the individual (e.g., from the cancer of the individual) comprises cancer cells that express HER2 on their cell surface at a level of IHC2+, as measured by IHC assay; and a sample obtained from the individual (e.g., from the cancer of the individual) comprises cancer cells that exhibit HER2 gene amplification, as measured by ISH assay (IHC2+/ISH-positive). In some embodiments, a sample obtained from the individual (e.g., from the cancer of the individual) is IHC3+. In some embodiments, a sample obtained from the individual (e.g., from the cancer of the individual) is IHC2+/ISH+.

In some embodiments, the cancer is HER2-positive LA/mBC. In some embodiments, the cancer is HER2-low LA/mBC. In some embodiments, the cancer is HER2-low LA/mGC/GEJC.

In some embodiments, the cancer is HER2-low (e.g., IHC2+/ISH-negative or IHC1+) second-line advanced gastric or gastroesophageal junction adenocarcinoma. In some embodiments, the cancer is HER2+(e.g., IHC2+/ISH+ or IHC3+) third-line or higher advanced breast cancer. In some embodiments, the cancer is HER2-low (e.g., IHC2+/ISH-negative or IHC1+) second- or third-line advanced breast cancer. In some embodiments, the cancer is HER2-low LA/mGC/GEJC. In some embodiments, the cancer is HER2+LA/mBC. In some embodiments, the cancer is HER2-low LA/mBC. In some embodiments, the cancer is previously treated advanced GC/GEJC or breast cancer.

In some embodiments, the individual has one or more visceral organ metastases. In some embodiments, the individual does not have one or more visceral organ metastases. In some embodiments, the individual has one or more non-visceral organ metastases. In some embodiments, visceral metastasis refers to tumor metastasis to the lungs, liver, kidneys, adrenal glands, heart, pleura, peritoneum, or other end organs in the chest, abdomen, and pelvis. In some embodiments, absence of visceral metastasis is defined as no tumor metastasis at these sites. In some embodiments, pleural effusion, abdominal effusion, lymph node metastasis, or bone metastasis are not defined as visceral metastases. In some embodiments, the cancer is breast cancer, e.g., locally advanced or metastatic breast cancer (LA/mBC). In some embodiments, the administration of tucatinib and the antibody-drug conjugate according to the methods of the present disclosure is a third-line or higher (3L+) treatment. In some embodiments, the individual has breast cancer, e.g., HER2+(e.g., IHC2+/ISH+ or IHC3+) third-line or higher advanced breast cancer.

In some embodiments, e.g., prior to treatment according to the methods disclosed herein, the individual has been previously treated for the cancer. In some embodiments, the cancer has previously been treated. For example, in some embodiments, the administration of tucatinib and the antibody-drug conjugate according to the methods of the present disclosure is a second-line (2L) treatment or a third-line or higher (3L+) treatment. In some embodiments, e.g., prior to treatment according to the methods disclosed herein, the individual has experienced progression on or after one or more standard of care therapies. In some embodiments, the individual is intolerant to one or more standard of care therapies.

In some embodiments, e.g., prior to treatment according to the methods disclosed herein, the individual is at least 18 years of age. In some embodiments, e.g., prior to treatment according to the methods disclosed herein, the individual has measurable disease according to RECIST v1.1 guidelines. In some embodiments, e.g., prior to treatment according to the methods disclosed herein, the individual has an Eastern Cooperative Oncology Group (ECOG) Performance Status score of 0 or 1. In some embodiments, e.g., prior to treatment according to the methods disclosed herein, the individual does not have uncontrolled cardiac disease, such as cardiac failure (e.g., New York Heart Association (NYHA) Class III or IV heart failure), cardiac arrhythmia (e.g., Grade 2 or higher arrhythmia or heart block), cardiac ischemia (e.g., unstable angina within the past 12 months, myocardial infarction or cerebral infarction within the past 6 months, etc.), or hypertension (e.g., uncontrolled hypertension (systolic blood pressure >180 mmHg and/or diastolic blood pressure >100 mmHg)). In some embodiments, e.g., prior to treatment according to the methods disclosed herein, the individual has a left ventricular ejection fraction (LVEF) ≥50% as assessed by echocardiogram (ECHO) or multigated acquisition scan (MUGA) within 4 weeks prior to treatment. In some embodiments, e.g., prior to treatment according to the methods disclosed herein, the individual has a Fridericia's corrected QT interval (QTcF)<470 ms. In some embodiments, e.g., prior to treatment according to the methods disclosed herein, the individual has one, some, or all of the following lab values: Hemoglobin (Hb) ≥9 g/dL; Absolute neutrophil count (ANC) ≥1.5×10⁹/L; Platelet count ≥100×10⁹/L; ALT and AST≤2.5×upper limit of normal (ULN) without liver metastases or ≤5×ULN with liver metastases; Serum total bilirubin ≤1.5×ULN or direct bilirubin ≤ULN for subjects with total bilirubin ≥1.5×ULN; serum total bilirubin ≤3×ULN for subjects with Gilbert's syndrome; and CrCl≥30 mL/min, as calculated using the Cockcroft-Gault formula or measured from a 24-hour urine collection.

In some embodiments, e.g., prior to treatment according to the methods disclosed herein, the individual has experienced disease progression on or after standard of care therapies, or is intolerant of standard of care therapies. In some embodiments, e.g., prior to treatment according to the methods disclosed herein, the individual has a cancer with RIC 1+ or higher expression of HER2.

In some embodiments, e.g., for individuals with breast cancer, the individual has a cancer with IHC1+ or IHC2+/ISH-negative status. In some embodiments, e.g., for individuals with breast cancer, the individual has received no more than 3 prior systemic cytotoxic chemotherapy regimens (including ADCs). In some embodiments, e.g., for individuals with breast cancer, the cancer has a BRCA mutation, and the individual has received prior treatment with a PARP inhibitor. In some embodiments, e.g., for individuals with breast cancer, the individual has experienced progression on or after, or is intolerant to, a treatment comprising a topoisomerase I inhibitor, trastuzumab deruxtecan (T-DXd), and/or sacituzumab govitecan. In some embodiments, e.g., for individuals with breast cancer, the cancer is hormone receptor-positive (HR+). In some embodiments, e.g., for individuals with HR+ breast cancer, the individual has progressed on endocrine therapy and/or received treatment comprising a CDK4/6 inhibitor.

In some embodiments, e.g., for individuals with breast cancer, the individual has a cancer with IHC3+ or IHC2+/ISH+ status. In some embodiments, e.g., for individuals with breast cancer, the individual has received prior treatment comprising trastuzumab, pertuzumab, and/or a taxane. In some embodiments, e.g., for individuals with breast cancer, the individual has experienced progression on or after, or is intolerant to, a treatment comprising a topoisomerase I inhibitor and/or trastuzumab deruxtecan (T-DXd).

In some embodiments, e.g., for individuals with gastric or GEJ cancer, the individual has a cancer with IHC1+ or IHC2+/ISH-negative status. In some embodiments, e.g., for individuals with gastric or GEJ cancer, the individual has received prior systemic therapy comprising platinum, fluorouracil, or taxane.

In some embodiments, e.g., prior to treatment according to the methods disclosed herein, the individual does not have CNS and/or leptomeningeal metastasis. In some embodiments, the individual has a treated CNS metastasis and the following apply: CNS metastases have been clinically stable for at least 4 weeks and baseline scans show no evidence of new or worsening CNS metastasis; Subject is on a stable dose of ≤10 mg/day of prednisone or equivalent for at least 2 weeks; and Subject does not have leptomeningeal metastasis. In some embodiments, e.g., prior to treatment according to the methods disclosed herein, the individual does not have ongoing sensory or motor neuropathy Grade 2 or higher. In some embodiments, e.g., prior to treatment according to the methods disclosed herein, the individual does not have acute, chronic, or symptomatic infection, including SARS-CoV-2 (except for subjects who have recovered clinically but continue to have a detectable presence of SARS-CoV-2), HIV, HBV, or HCV infection (unless subjected to curative treatment for HBV/HCV if documented sustained virologic response of ≥12 weeks or HBV negative DNA of ≥12 weeks). In some embodiments, e.g., prior to treatment according to the methods disclosed herein, the individual does not have history of idiopathic pulmonary fibrosis, organizing pneumonia, drug-induced pneumonitis/interstitial lung disease (ILD), or idiopathic pneumonitis/ILD. In some embodiments, e.g., prior to treatment according to the methods disclosed herein, the individual does not have history of another invasive malignancy within 3 years before the first dose, except for adequately resected early-stage non-melanoma skin cancer or carcinoma in situ and history of prostate cancer (T2NXMX or lower with Gleason score ≤7) treated with definitive intent (surgically or with radiation therapy) at least 1 year prior, provided that subjects who have undergone radical prostatectomy must have undetectable prostate-specific antigen (PSA) for >1 year and at screening and subjects who have had radiation must have a PSA doubling time >1 year (based on at least 3 values determined >1 month apart) and a total PSA value that does not meet Phoenix criteria for biochemical recurrence (ie, <2.0 ng/mL above nadir). In some embodiments, e.g., prior to treatment according to the methods disclosed herein, the individual does not have uncontrolled cardiac disease. In some embodiments, e.g., prior to treatment according to the methods disclosed herein, the individual has not received prior MMAE treatment, major surgery within 4 weeks prior to Cycle 1 Day 1, chronic oxygen therapy or has Grade ≥3 pulmonary disease unrelated to underlying malignancy, live or live-attenuated vaccine within 30 days, whole blood or plasma transfusions within 14 days prior to the Cycle 1 Day 1, or have used strong cytochrome P450 (CYP)2C8 inhibitor within 5 half-lives of the inhibitor or have used a strong CYP2C8 or CYP3A4 inducer within 5 days prior to first dose. In some embodiments, e.g., prior to treatment according to the methods disclosed herein, the individual does not have bowel obstruction, history or presence of inflammatory enteropathy or extensive intestinal resection (hemicolectomy or extensive small intestine resection with chronic diarrhea) or moderate to severe chronic diarrhea or any conditions may alter oral drug absorption.

### Administration

In some embodiments, the antibody-drug conjugate is administered to the individual at a dose of 1.0 mg/kg, 1.25 mg/kg, or 1.5 mg/kg, optionally with a maximum dose of 150 mg. In some embodiments, the antibody-drug conjugate is administered to the individual at a dose of 1.0 mg/kg, optionally with a maximum dose of 150 mg. In some embodiments, the antibody-drug conjugate is administered to the individual at a dose of 1.25 mg/kg, optionally with a maximum dose of 150 mg. In some embodiments, the antibody-drug conjugate is administered to the individual at a dose of 1.5 mg/kg, optionally with a maximum dose of 150 mg. In some embodiments, the antibody-drug conjugate is administered intravenously. In some embodiments, the antibody-drug conjugate is administered to the individual every 2 weeks or every 14 days (Q2W). In some embodiments, the antibody-drug conjugate is disitamab vedotin, and DV is administered to the individual intravenously (IV) at a dose of 1.5 mg/kg, optionally with a maximum dose of 150 mg, every 2 weeks or every 14 days (Q2W). In some embodiments, the antibody-drug conjugate is administered to the individual on Days 1, 15, and 29 of a 6-week cycle.

In some embodiments, tucatinib is administered to the individual at a dose of between about 200 mg and about 300 mg. In some embodiments, tucatinib is administered to the individual at a dose of between 200 mg and 300 mg. In some embodiments, tucatinib is administered to the individual at a dose of 200 mg, 250 mg, or 300 mg. In some embodiments, 300 mg tucatinib is administered to the individual. In some embodiments, 200 mg tucatinib is administered to the individual. In some embodiments, 250 mg tucatinib is administered to the individual. In some embodiments, tucatinib is administered orally to the individual. In some embodiments, tucatinib is administered to the individual twice daily (BID). In some embodiments, 300 mg tucatinib is orally administered to the individual twice daily (BID). In some embodiments, 200 mg tucatinib is orally administered to the individual twice daily (BID). In some embodiments, 250 mg tucatinib is orally administered to the individual twice daily (BID). In some embodiments, tucatinib is administered twice daily starting at Day 8 of a 6-week cycle. In some embodiments, the antibody-drug conjugate is disitamab vedotin, DV is administered to the individual intravenously (IV) at a dose of 1.0 mg/kg, 1.25 mg/kg, or 1.5 mg/kg, optionally with a maximum dose of 150 mg, every 2 weeks or every 14 days (Q2W), and 300 mg tucatinib is orally administered to the individual twice daily (BID). In some embodiments, the antibody-drug conjugate is disitamab vedotin, DV is administered to the individual intravenously (IV) at a dose of 1.0 mg/kg, optionally with a maximum dose of 150 mg, every 2 weeks or every 14 days (Q2W), and 300 mg tucatinib is orally administered to the individual twice daily (BID). In some embodiments, the antibody-drug conjugate is disitamab vedotin, DV is administered to the individual intravenously (IV) at a dose of 1.25 mg/kg, optionally with a maximum dose of 150 mg, every 2 weeks or every 14 days (Q2W), and 300 mg tucatinib is orally administered to the individual twice daily (BID). In some embodiments, the antibody-drug conjugate is disitamab vedotin, DV is administered to the individual intravenously (IV) at a dose of 1.5 mg/kg, optionally with a maximum dose of 150 mg, every 2 weeks or every 14 days (Q2W), and 300 mg tucatinib is orally administered to the individual twice daily (BID).

In some embodiments, the antibody-drug conjugate is disitamab vedotin, DV is administered to the individual intravenously (IV) at a dose of 1.0 mg/kg, 1.25 mg/kg, or 1.5 mg/kg, optionally with a maximum dose of 150 mg, every 2 weeks or every 14 days (Q2W), and 250 mg tucatinib is orally administered to the individual twice daily (BID). In some embodiments, the antibody-drug conjugate is disitamab vedotin, DV is administered to the individual intravenously (IV) at a dose of 1.0 mg/kg, 1.25 mg/kg, or 1.5 mg/kg, optionally with a maximum dose of 150 mg, every 2 weeks or every 14 days (Q2W), and 200 mg tucatinib is orally administered to the individual twice daily (BID). In some embodiments, DV is administered to the individual intravenously (IV) at a dose of 1.0 mg/kg, 1.25 mg/kg, or 1.5 mg/kg, optionally with a maximum dose of 150 mg, on Days 1, 15, and 29 of a 6-week cycle, and 300 mg tucatinib is orally administered to the individual twice daily (BID) starting at Day 8 of the 6-week cycle.

In some embodiments, the administration of tucatinib and the antibody-drug conjugate according to the methods of the present disclosure is a second-line (2L) treatment. In some embodiments, the administration of tucatinib and the antibody-drug conjugate according to the methods of the present disclosure is a third-line or higher (3L+) treatment.

In some embodiments, administration of tucatinib and the anti-HER2 antibody-drug conjugate according to the methods described herein results in increased internalization of the antibody-drug conjugate by cells of the cancer, e.g., as compared to administration of the antibody-drug conjugate in the absence of tucatinib.

In some embodiments, administration of tucatinib and the anti-HER2 antibody-drug conjugate according to the methods described herein results in increased expression of HER2 by cells of the cancer, e.g., as compared to administration of the antibody-drug conjugate in the absence of tucatinib. For example, in some embodiments, administration of tucatinib and the antibody-drug conjugate results in increased total expression of HER2 by cells of the cancer, e.g., as compared to administration of the antibody-drug conjugate in the absence of tucatinib. In some embodiments, administration of tucatinib and the anti-HER2 antibody-drug conjugate results in increased surface expression of HER2 by cells of the cancer, e.g., as compared to administration of the antibody-drug conjugate in the absence of tucatinib. Assays for measuring internalization of HER2 or total or surface expression of HER2 in cells are known in the art, and exemplary methods are described herein. For example, in some embodiments, HER2 internalization is measured using fluorescence microscopy, e.g., as exemplified infra.

In some embodiments, administration of tucatinib and the anti-HER2 antibody-drug conjugate according to the methods described herein changes the dwell time of HER2 at the cell surface, e.g., as compared to administration of the antibody-drug conjugate in the absence of tucatinib. As used herein, the term “dwell time” refers to an amount of time that a protein positions at the surface of the cell. In some embodiments of any of the methods described herein, the administration of the tucatinib, or a salt or solvate thereof, increases the dwell time of HER2 at the cell surface. In some embodiments of any of the methods described herein, the administration of tucatinib, or salt or solvate thereof, changes an internalization of membrane-bound HER2. In some embodiments of any of the methods described herein, the administration of tucatinib, or salt or solvate thereof, increases an internalization of membrane-bound HER2. In some embodiments of any of the methods described herein, the administration of the tucatinib, or salt or solvate thereof, changes a lysosomal degradation of HER2. In some embodiments of any of the methods described herein, the administration of the tucatinib, or salt or solvate thereof, increases a lysosomal degradation of HER2.

In some embodiments, administration of tucatinib and the antibody-drug conjugate results in a complete response (CR) or partial response (PR) in the individual. In some embodiments, response to treatment according the methods disclosed herein is evaluated using RECIST v1.1 criteria. Exemplary criteria are provided below.

Response Evaluation Criteria for Solid Tumors (RECIST) Version 1.1
(Eisenhauer, E. A. et al. (2009) Eur J Cancer 45(2): 228-247)

| Term | Definition |
|---|---|
| Complete Response | Disappearance of all target lesions. Any pathological lymph |
| (CR) | nodes (whether target or non-target) must have reduction in |
| | short axis to <10 mm |
| | Cannot have previously met criteria for PD |
| | Confirmation required with repeat scans at least 4 weeks |
| | after first documentation of response |
| Partial Response | A ≥30% decrease in the sum of diameters of target lesions |
| (PR) | (longest for non-nodal target lesions and the short axes for |
| | nodal target lesions), taking as reference the baseline sum |
| | diameters |
| | Cannot have previously met criteria for PD |
| | Confirmation required with repeat scans at least 4 weeks |
| | after first documentation of response |
| Stable Disease (SD) | Neither sufficient shrinkage to qualify for PR nor sufficient |
| | increase to qualify for PD, taking as reference the smallest |
| | sum diameters while on study |
| | Cannot have previously met criteria for PD |
| | Confirmation is not required |
| Progressive Disease | A ≥20% relative increase in the sum of diameters of target |
| (PD) | lesions, taking as reference the smallest sum on study |
| | (including baseline sum if that is the smallest on study). In |
| | addition, the sum must also demonstrate an absolute increase ≥5 mm |
| | The appearance of 1 or more new lesions is also considered |
| | progression |
| | Confirmation is not required unless equivocal |

In some embodiments, the individual is a human.

### III. Kits and Articles of Manufacture

In another aspect, an article of manufacture or kit is provided which comprises an anti-HER2 antibody-drug conjugate as described herein. The article of manufacture or kit may further comprise instructions for use of the antibody-drug conjugate in the methods of the present disclosure, e.g., for administering an effective amount of tucatinib and the antibody-drug conjugate to an individual in need thereof according to any one of the methods disclosed herein. In some embodiments, the article of manufacture or kit further comprises tucatinib. Thus, in certain embodiments, the article of manufacture or kit comprises instructions for the use of treating or preventing progression of cancer in an individual, e.g., according to any one of the methods disclosed herein.

The article of manufacture or kit may further comprise a container. Suitable containers include, for example, bottles, vials (e.g., dual chamber vials), syringes (such as single or dual chamber syringes) and test tubes. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for intravenous or other modes of administration. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

In a specific embodiment, the present invention provides kits for a single dose-administration unit. Such kits comprise a container of an aqueous formulation of therapeutic antibody, including both single or multi-chambered pre-filled syringes. Exemplary pre-filled syringes are available from Vetter GmbH, Ravensburg, Germany.

The article of manufacture or kit herein optionally further comprises a container comprising a second medicament, e.g., tucatinib.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: In Vitro Cytotoxicity of Tucatinib in Combination with Disitamab Vedotin

Disitamab vedotin (DV, RC48-ADC, Hertuzumab-MMAE) is an antibody-drug conjugate (ADC) that targets cancers expressing HER2, an oncogenic growth factor receptor that promotes cell proliferation and survival. DV consists of an anti-HER2 monoclonal antibody disitamab (RC48) conjugated with 4 molecules (on average) of the tubulin-disrupting anti-mitotic agent monomethyl auristatin E (MMAE) via a cleavable peptide linker. DV has multimodal antitumor mechanisms of action that include direct cytotoxicity of HER2-expressing cancer cells and bystander effect based-cytotoxicity of neighboring cells, both of which are mediated by the intracellular release of MMAE within the targeted cell. Released MMAE can induce immunogenic cell death (ICD), which promotes immune cell recruitment to the tumor. In addition, DV stimulates Fc-gamma receptor mediated antibody-dependent cellular cytotoxicity (ADCC), which can lead to target cell death. DV also inhibits HER2-activated downstream signaling pathways, further blocking cell growth and proliferation.

To test whether enhanced antitumor activity could be achieved by dual HER2 targeting with DV in combination with tucatinib, the cytotoxicity of the combination was investigated in breast and gastric cancer cell lines with a range of HER2 cell surface expression levels (5×10⁴-1.5×10⁶ copies/cell), which were determined using quantitative flow cytometry (qFACS).

### Materials and Methods

qFACS Analysis of Cell Surface HER2 Density

HER2 cell surface levels were determined by qFACS analysis. Briefly, cells were stained with an anti-HER2 antibody (R&D Systems, Inc.; #MAB1129) and Zombie Aqua (BioLegend, Inc.; #423101) for dead cell exclusion. Cells were fixed, and HER2 receptor copy number per cell was quantified using a QIFIKIT (Agilent Dako) per the manufacturer's protocol.

### Cytotoxicity Assays

The following cell lines were cultured in the vendor-recommended conditions: breast cancer cell lines SK-BR-3, BT-474, HCC-202, EFM-192C, BT-483, and CAMA-1, and gastric cancer cell lines NCI-N87, OE19, and RERF-GC-1B. Cytotoxicity assays were performed using CellTiter-Glo (CTG) Luminescent Cell Viability assay (Promega Corporation) as per the manufacturer's protocol. Cells were seeded in 96-well plates and incubated overnight as previously described (Kulukian, A. et al. (2020) Mol Cancer Ther. 19(4):976-987). Cells were dosed in triplicate with combinations of three-fold increasing concentrations of DV (0.12-6400 ng/mL) and three-fold increasing concentrations of tucatinib (4.6-120 nM). Cells were then incubated at 37° C. in 5% C02, and cell viability was measured at 96 hours after dosing.

### Drug Combination Computational Analysis

Drug combination computational analysis was performed to determine whether tucatinib and DV in combination showed an additive or possibly synergistic cytotoxic effect on HER2-positive breast and gastric cancer cell lines. Data for single-agent activity, or single-agent plus a fixed dose of a second agent, were analyzed in Prism (GraphPad Software). Data reflecting the full matrix of dose combinations of both agents were analyzed to assess synergy or antagonism of each combination relative to the Highest Single Agent (HSA) additivity model, using a previously described analytical and significance framework (2). Briefly, additivity models such as HSA give a prediction of additive activity (that is, neither synergy nor antagonism) for each dose combination in the matrix, based on the single-agent activity of both drugs. Heatmap visualizations were used to depict the deviations of the observed viability for a given dose combination from the additive model predictions. Statistical significance of deviations from additivity was assessed using t-tests of model predictions minus observed viability. To increase power and reward consistency, tests were performed in 3×3 blocks of contiguous dose combinations. All 3×3 dose blocks tiling the dose combination space were tested, and blocks that tested as significant at a P-value of 0.01, adjusted for multiple testing by the total number of 3×3 blocks, in either direction (synergy or antagonism) were marked with rectangle boxes.

### Results

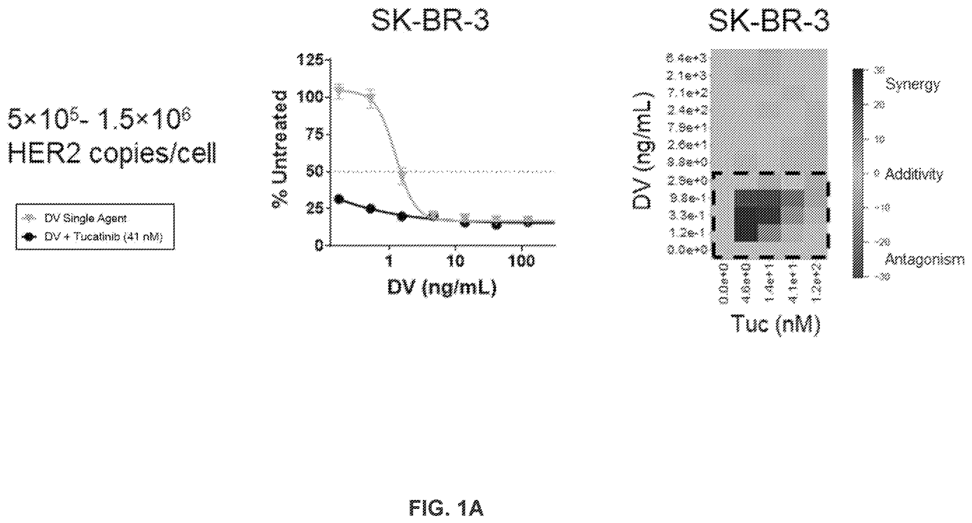

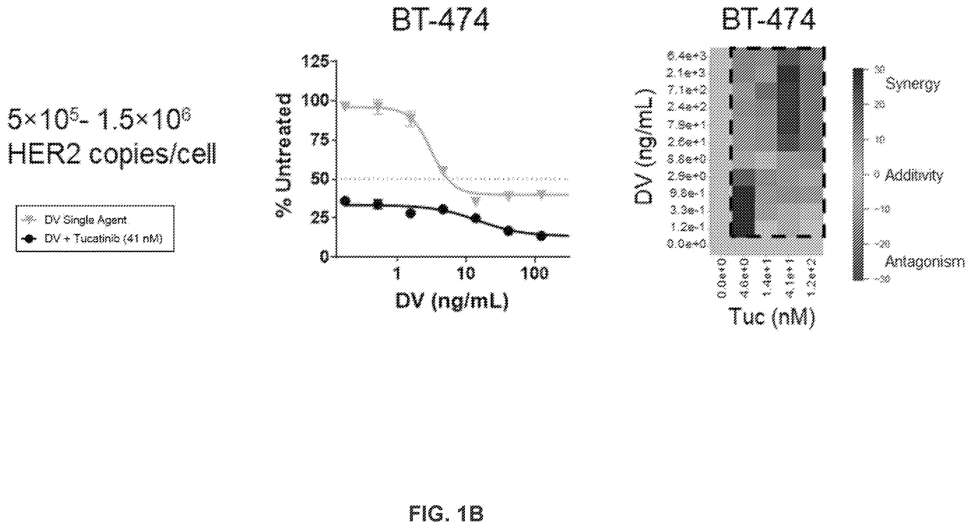

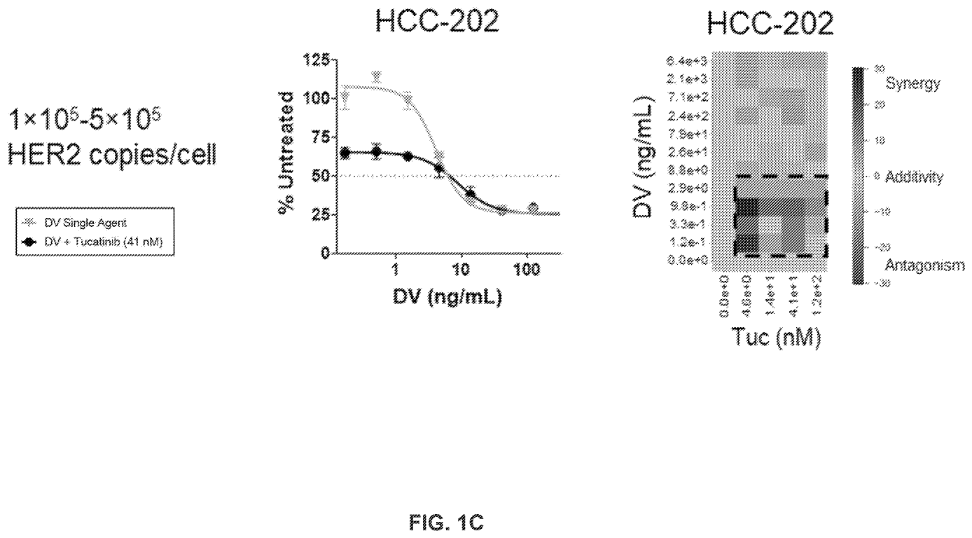

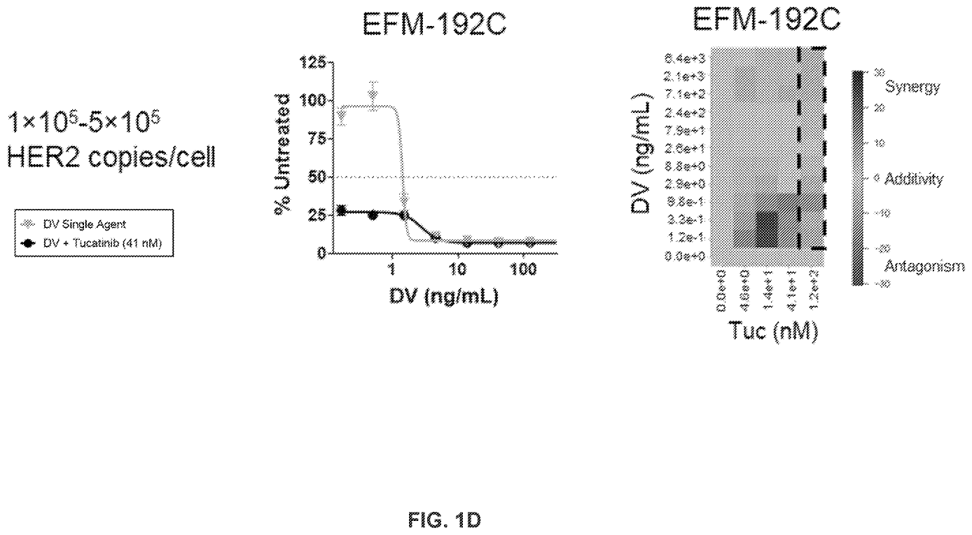

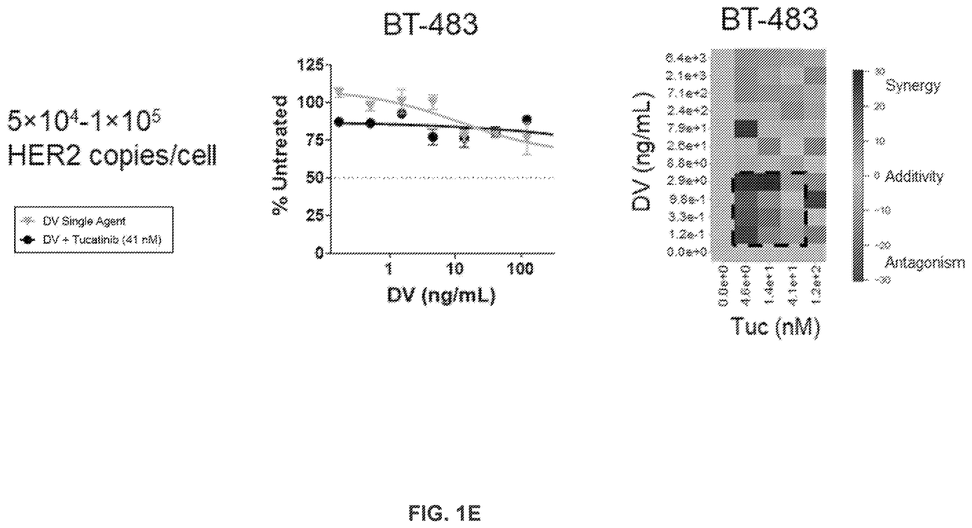

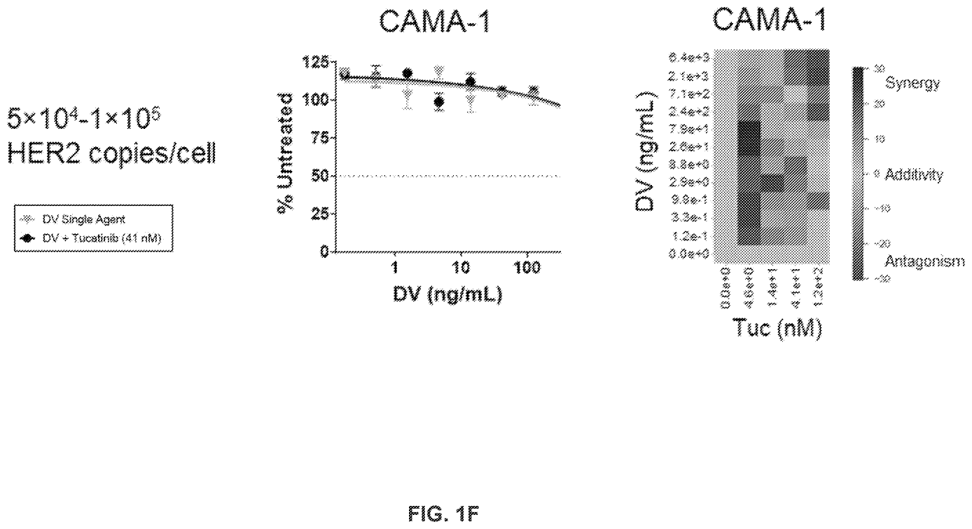

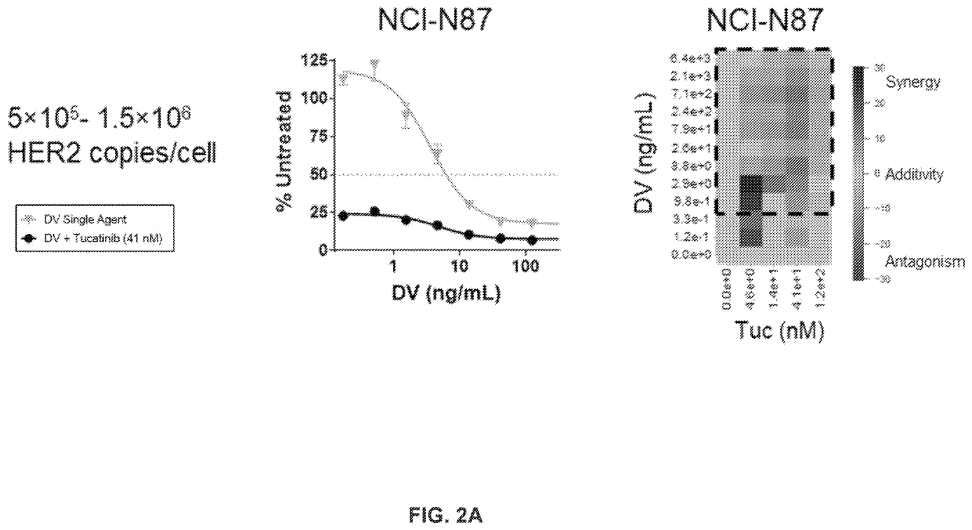

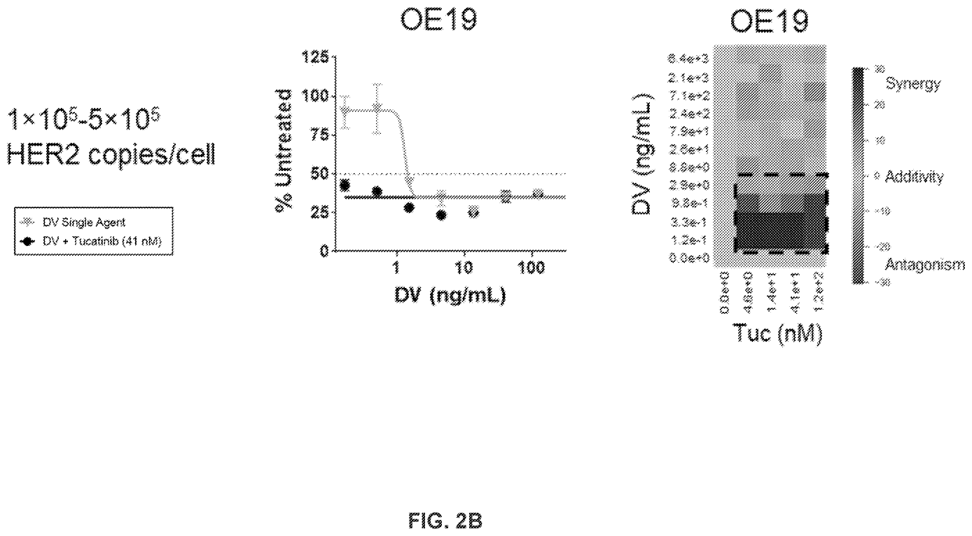

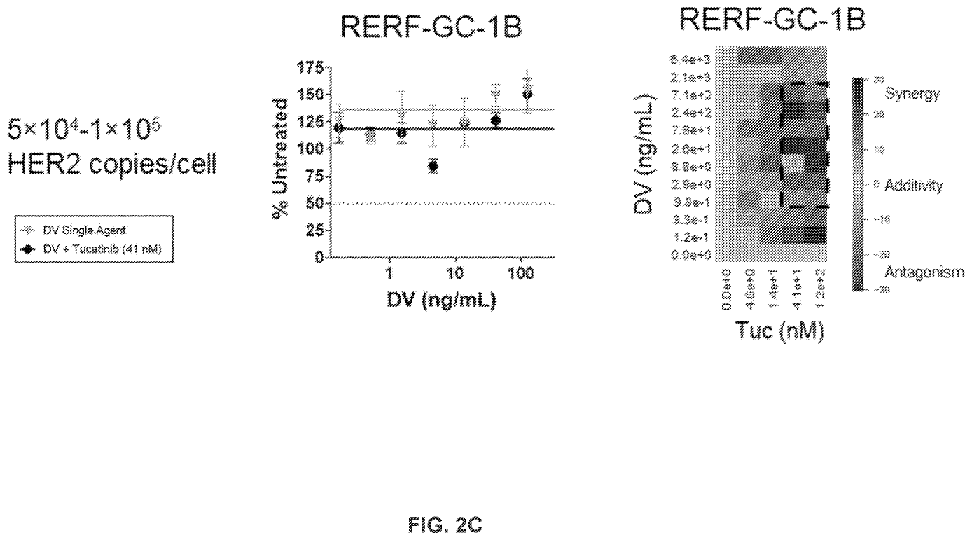

In vitro cytotoxicity assays showed that the combination of tucatinib with DV resulted in improved anti-tumor activity in breast and gastric cancer models with variable HER2 expression. Cytotoxicity curves of DV alone or in combination with a clinically relevant dose of tucatinib (41 nM) are shown in FIGS. 1A-1F for breast cancer cell lines and FIGS. 2A-2C for gastric cancer cell lines. The cytotoxicity results demonstrated increased antiproliferative activity for the combination of DV and tucatinib (41 nM) compared to DV as a single agent in HER2-positive breast and gastric cancer cell lines SK-BR-3, BT-474, HCC-202, EFM-192C, NCI-N87, and OE19.

Drug combination computational analysis of the cytotoxicity data in HER2+ breast (FIGS. 1A-1F) and gastric cancer cell lines (FIG. 2A-2C) showed enhanced cytotoxicity of the DV+tucatinib combination over DV as a single agent. The heatmaps indicate model predictions minus observed viability for each dose combination based on the HSA additivity model. Synergistic activity was observed in all tested breast and gastric cancer cell lines except for CAMA-1.

### Example 2: Internalization of Disitamab in the Presence of Tucatinib

As part of the mechanistic understanding of the enhanced cytotoxicity of the DV+tucatinib combination, additional experiments were performed to test if tucatinib could increase HER2 levels in cancer cell lines, since other ErbB family-targeting TKIs have been reported to modulate HER2 protein levels and cell-surface localization (Singla, H. and Munshi, A. (2020) Crit Rev Oncol 25(3):241-250). qFACS analysis showed increased HER2 levels across breast and gastric cancer cell lines with variable HER2 expression. To test whether increased HER2 levels result in increased DV internalization, internalization studies were performed using disitamab, the antibody backbone of DV, in the presence and absence of tucatinib. This Example demonstrates that tucatinib has the potential to increase HER2 levels across indications, thereby driving enhanced internalization of DV.

### Materials and Methods

### Internalization Assays

The internalization experiments were conducted by detecting an antibody-bound pH sensitive probe using automated time-lapse microscopy. Briefly, breast cancer cell lines SK-BR-3, BT-474, HCC-202, and EFM-192C and gastric cancer cell lines NCI-N87 and OE19 were seeded in 96-well flat clear-bottom black tissue culture-treated microplates (Coming, cat #3603). Cells were plated at 1.5×10⁴-2.5×10⁴ cells/200 μL/well in growth medium and allowed to adhere overnight at 37° C. Disitamab and isotype control antibody (human IgG) were labeled with IncuCyte Fabfluor-pH Red Antibody Labeling Reagent (Essen Bioscience, cat #4722) according to the manufacturer's protocol. Volumes of the test antibodies, Fabfluor reagent and medium required were calculated at 2× the final assay concentration, and the Fabfluor reagent was added at a molar ratio of 1:3 to the antibody. Antibody and Fabfluor were mixed gently and incubated at 37° C. for 15 minutes. Media containing Fabluor-labeled disitamab (1 g/mL final concentration) with or without tucatinib (100 nM final concentration) was added to cells. The plates were arranged on microplate trays in the IncuCyte SX3 system (Essen Bioscience), and scans were acquired using the Standard protocol. Phase data and red channel data (acquisition time set to 400 ms) were collected, with 4 images per well every 30 minutes for the first 6 hours and every 2 hours after that for up to 72.5 hours with the objective set at 10×. Quantification of fluorescence signal intensity was performed using the IncuCyte software analysis tool. Cell surface HER2 density was quantified using qFACS analysis as described supra.

### Analysis Pipeline

The analysis was refined and tuned per cell line utilizing phase area confluence and manual image selection for preview and training of the algorithm. Upon completion of the analysis, red object integrated intensity (RCU×μm²/image) data were graphed using GraphPad Prism. The experiment was performed in duplicate (technical replicates) for two biological replicates.

### Results

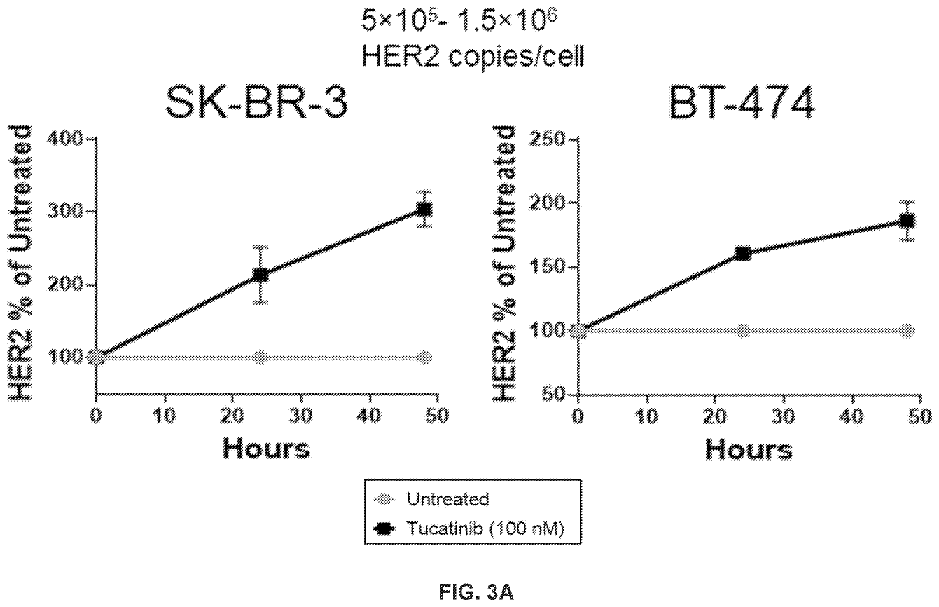

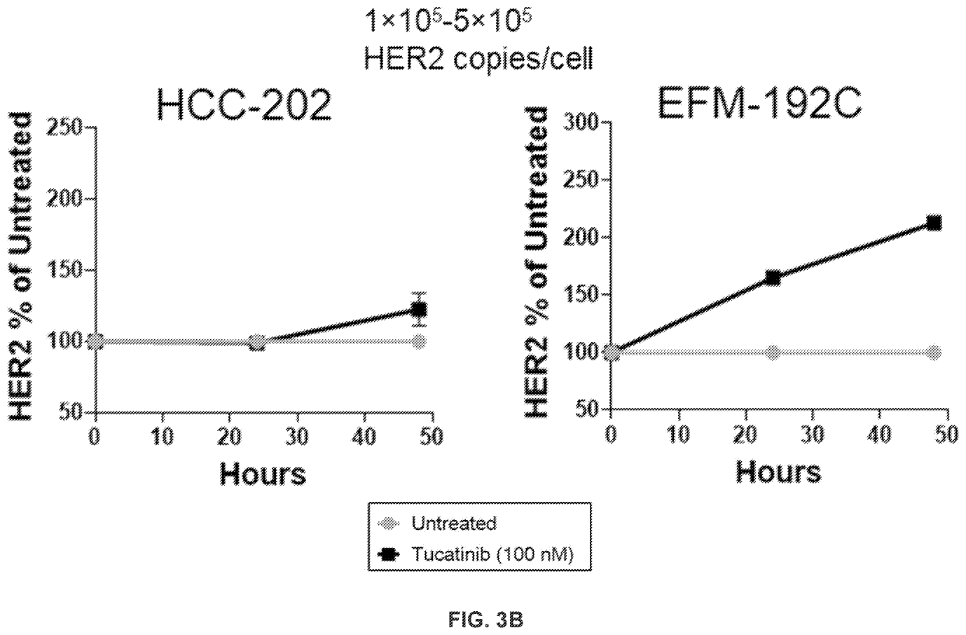

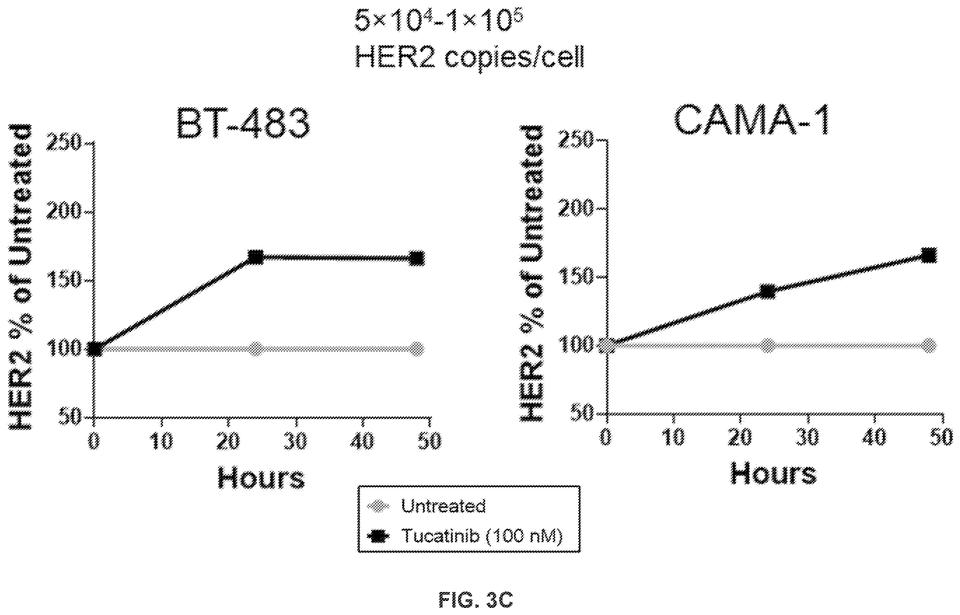

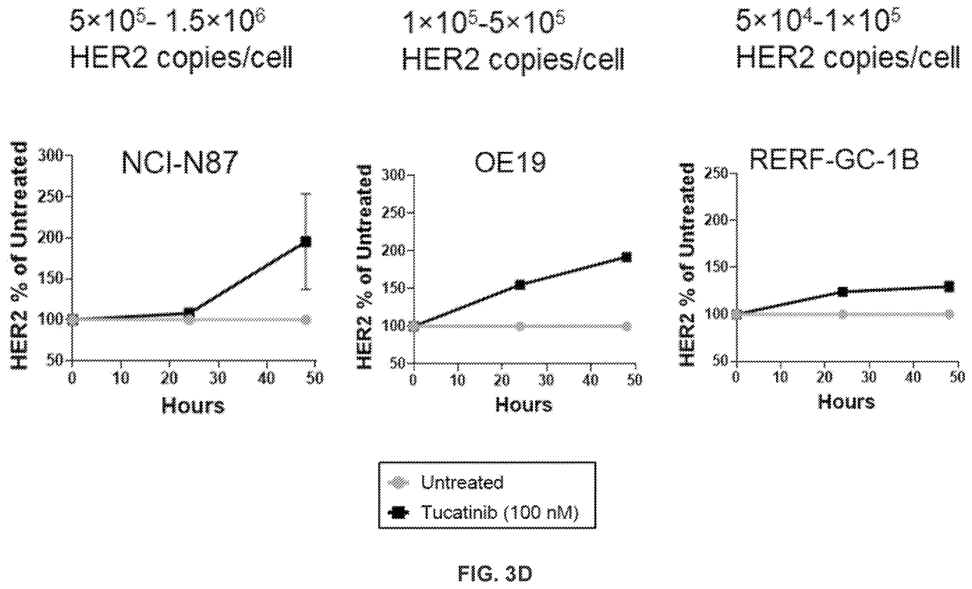

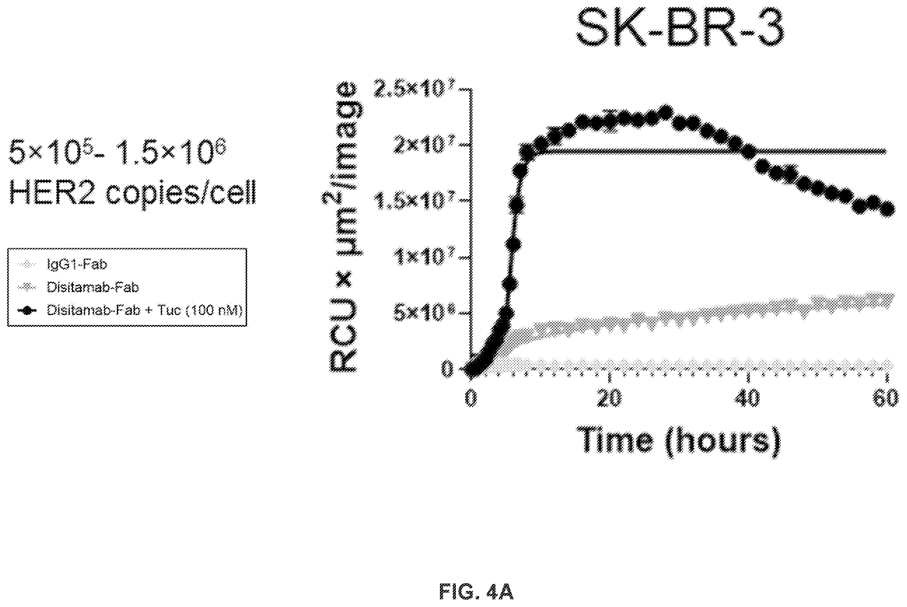

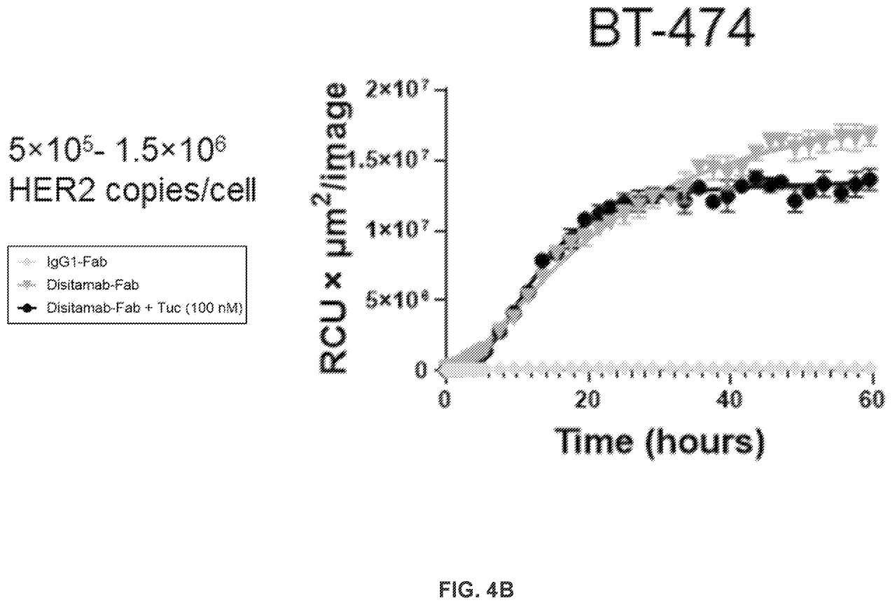

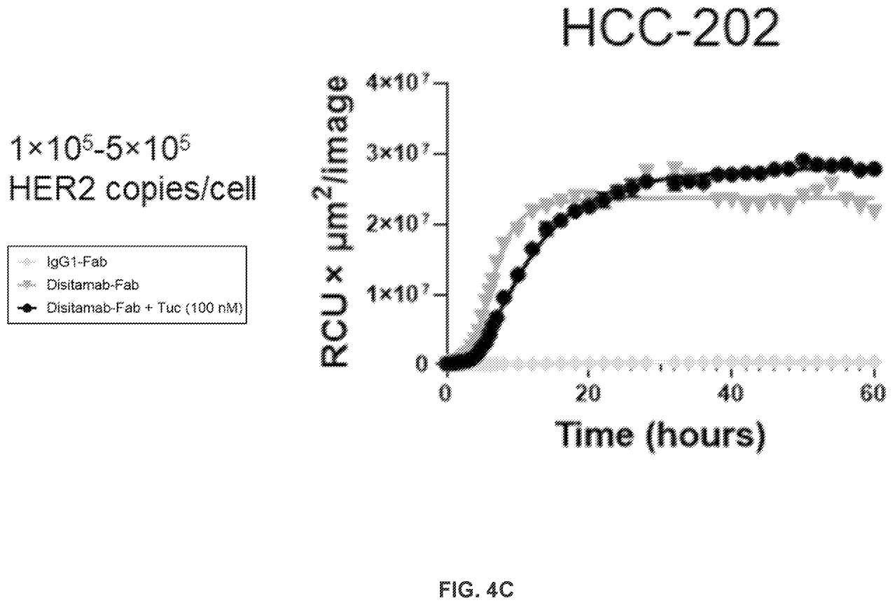

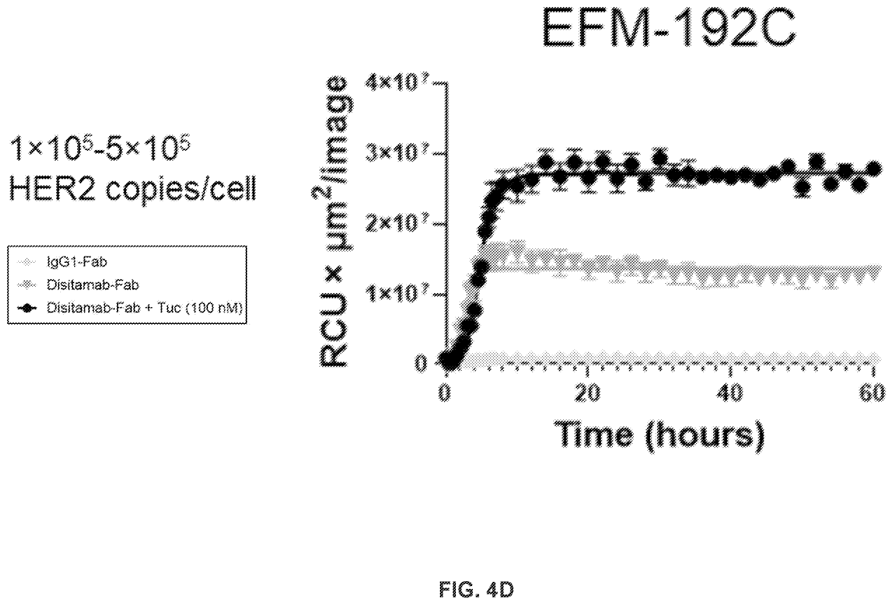

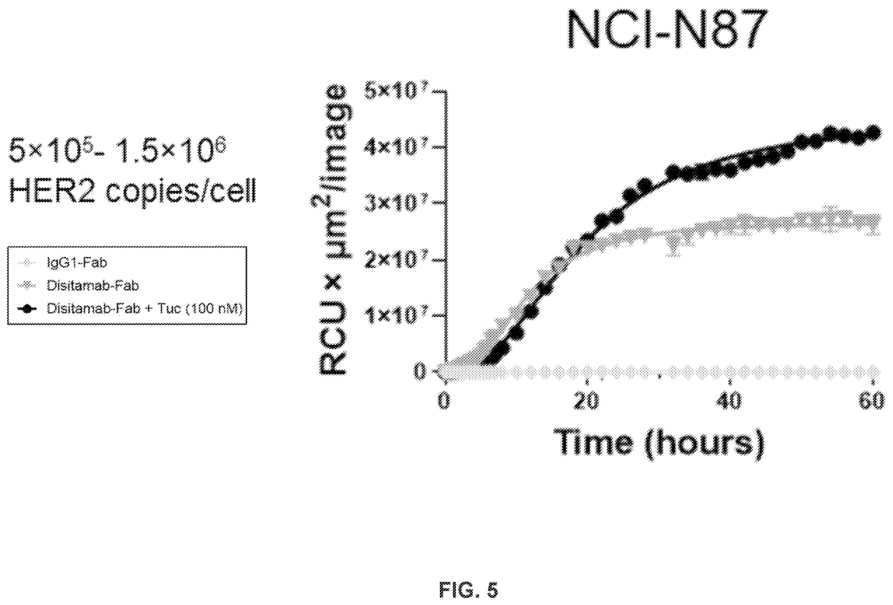

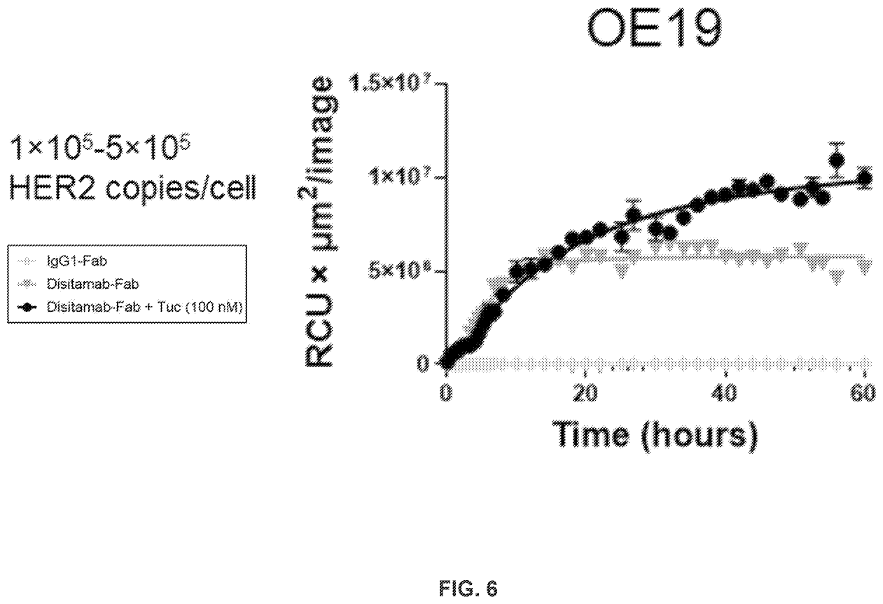

qFACS analysis of SK-BR-3, BT-474, HCC-202, EFM-192C, BT-483, CAMA-1, NCI-N87, OE19, and RERF-GC-1B cancer cell lines treated with 100 nM tucatinib showed an increase in cell-surface HER2 levels over time compared with untreated cells (FIGS. 3A-3D). Furthermore, increased disitamab internalization in the presence of 100 nM tucatinib compared to disitamab alone was observed in breast cancer cell lines SK-BR-3, EFM-192C, NCI-N87, and OE19 (FIGS. 4A-4D); and gastric cancer cell lines NCI-N87 and OE19 (FIGS. 5 & 6). These results support a model by which the increase in HER2 levels mediated by tucatinib drives increased antitumor activity when tucatinib is combined with the HER2-targeted ADC DV.

### Example 3: Internalization and Single-Agent Cytotoxicity of DV in HER2-Expressing Target Cells

Other antibody-drug conjugate drugs that target cancers expressing HER2 exist as therapeutic options for treatment of HER2+ solid tumor malignancies, including trastuzumab emtansine (T-DM1). However, potent cytotoxicity for these drugs often requires high expression of HER2 in the target cells. This Example demonstrates increased internalization of disitamab, the antibody backbone of DV, against a panel of breast and gastric cancer cell lines with variable HER2 expression, including within the HER2-low expression range, over the internalization observed with trastuzumab, the antibody backbone of T-DM1.

### Materials and Methods

### Internalization Assays

The internalization experiments were conducted by detecting an antibody-bound pH sensitive probe using automated time-lapse microscopy. Briefly, breast cancer cell lines SK-BR-3, BT-474, HCC-202, EFM-192C, BT-483, and CAMA-1 and gastric cancer cell lines NCI-N87, OE19, and RERF-GC-1B were seeded in 96-well flat clear-bottom black tissue culture-treated microplates (Corning, cat #3603). Cells were plated at 1.5×10⁴-2.5×10⁴ cells/200 μL/well in growth medium and allowed to adhere overnight at 37° C. Disitamab, trastuzumab, and isotype control antibody (human IgG) were labeled with IncuCyte Fabfluor-pH Red Antibody Labeling Reagent (Essen Bioscience, cat #4722) according to the manufacturer's protocol. Volumes of the test antibodies, Fabfluor reagent and medium required were calculated at 2× the final assay concentration, and the Fabfluor reagent was added at a molar ratio of 1:3 to the antibody. Fabfluor dye fluoresces only upon exposure to an acidified compartment, namely endocytic vesicles and lysosomes, signifying internalization and intracellular processing of the ADC. Antibody and Fabfluor were mixed gently and incubated at 37° C. for 15 minutes. Media containing Fabfluor-labeled disitamab or trastuzumab (1 g/mL final concentration) was added to cells. The plates were arranged on microplate trays in the IncuCyte SX3 system (Essen Bioscience), and scans were acquired using the Standard protocol. Phase data and red channel data (acquisition time set to 400 ms) were collected, with 4 images per well every 30 minutes for the first 6 hours and every 2 hours after that for up to 40 hours with the objective set at 10×. Quantification of fluorescence signal intensity was performed using the IncuCyte software analysis tool.

### Cytotoxicity Assays

The following cell lines were cultured in the vendor-recommended conditions: breast cancer cell lines SK-BR-3, BT-474, UACC-893, HCC-202, JIMT-1, and BT-483. Cytotoxicity assays were performed using CellTiter-Glo (CTG) Luminescent Cell Viability assay (Promega Corporation) as per the manufacturer's protocol. Cells were seeded in 96-well plates and incubated overnight as previously described (1). Cells were dosed in triplicate with combinations of three-fold increasing concentrations of DV (0.12-6400 ng/mL) and three-fold increasing concentrations of tucatinib (4.6-120 nM). Cells were then incubated at 37° C. in 5% CO₂, and cell viability was measured at 96 hours after dosing.

### Results

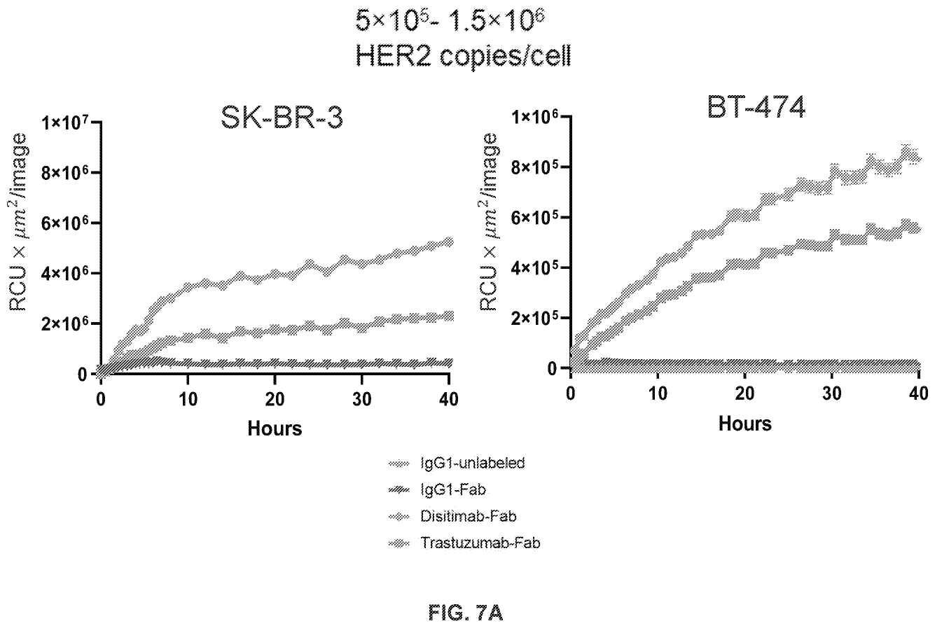

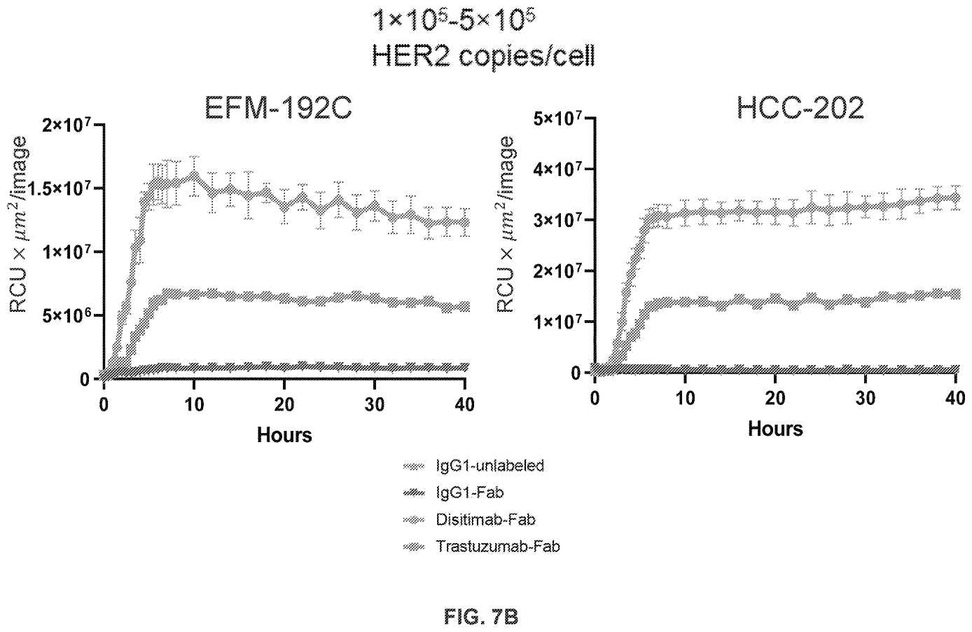

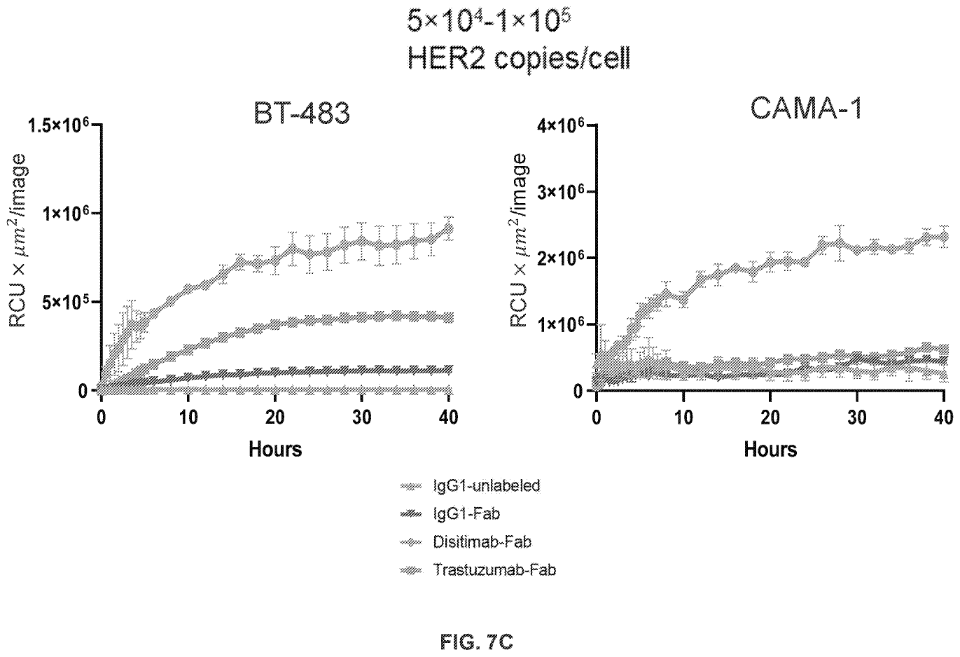

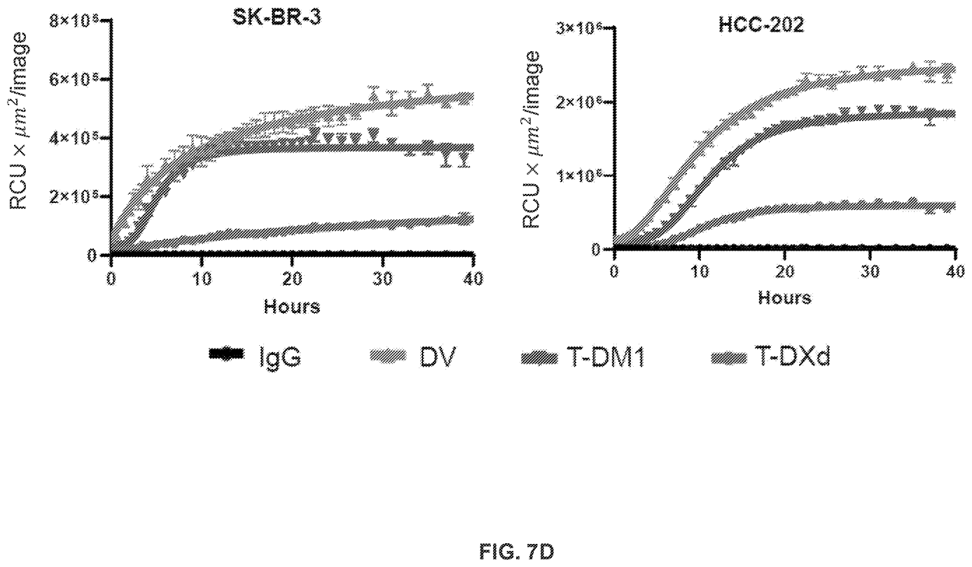

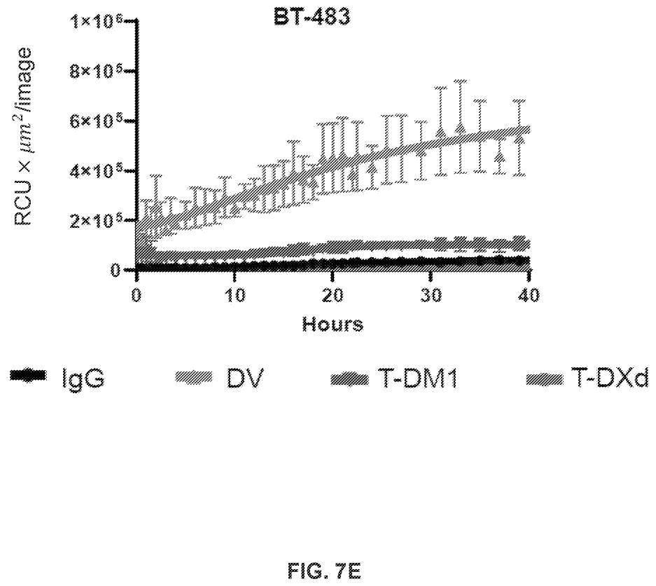

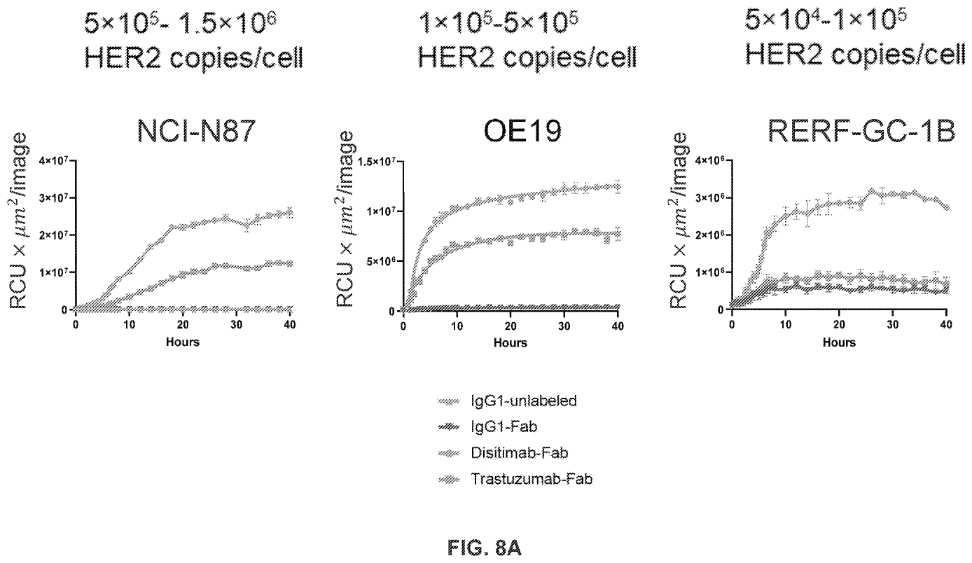

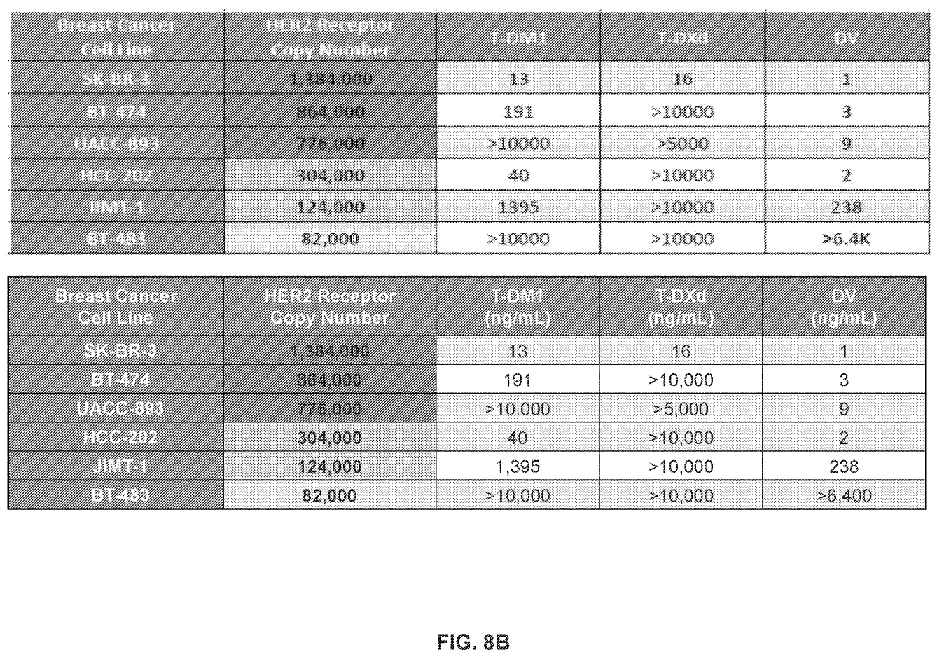

Increased disitamab internalization as compared to trastuzumab was observed in breast cancer cell lines SK-BR-3, BT-474, EFM-192C, HCC-202, BT-483, and CAMA-1 (FIGS. 7A-7C) and gastric cancer cell lines NCI-N87, OE19, and RERF-GC-1B (FIG. 8A).

Internalization of anti-HER2 ADCs DV, T-DM1, and T-DXd (two HER2-targeting ADCs with trastuzumab as a backbone) was also tested in breast cancer cell lines SK-BR-3, HCC-202, and BT-483. DV had enhanced internalization compared to T-DM1 and T-DXd (FIGS. 7D & 7E). However, differences in ADC formulation buffer may have impacted the fluorescent labeling of DV, T-DM1, and T-DXd, and as such these results should be considered preliminary until confirmed by orthogonal assays.DV, T-DM1, and T-DXd were screened in a panel of breast cancer cell lines spanning a range of HER2 expression (calculated by quantitative flow cytometry for surface expression). Drugs were titrated across a range of 0.01-10,000 ng/mL in 96-hour CellTiterGlo cytotoxicity assays, and data were analyzed by Prism (GraphPad), and IC50 values (concentration required for 50% inhibition) were generated from best-fit curves.

DV demonstrated in vitro cytotoxic activity against the panel of breast cancer cell lines with variable HER2 expression, including within the HER2-low expression range (FIG. 8B). The IC50 observed for DV was lower than the comparable IC50 for T-DM1 and T-DXd for each cell line.

### Example 4: Phase 1b/2 Open-Label Study of Disitamab Vedotin Monotherapy or in Combination with Tucatinib in Solid Tumors

Clinical success has been seen in several trials combining tucatinib with the HER2-targeting mAb trastuzumab, but tucatinib has not been paired previously with a vedotin-conjugated ADC.

### Design

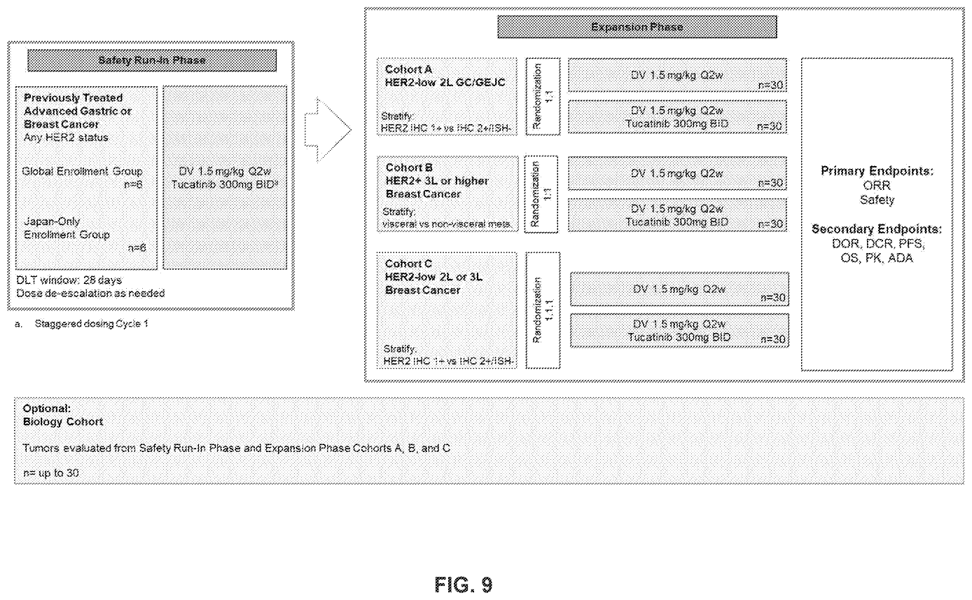

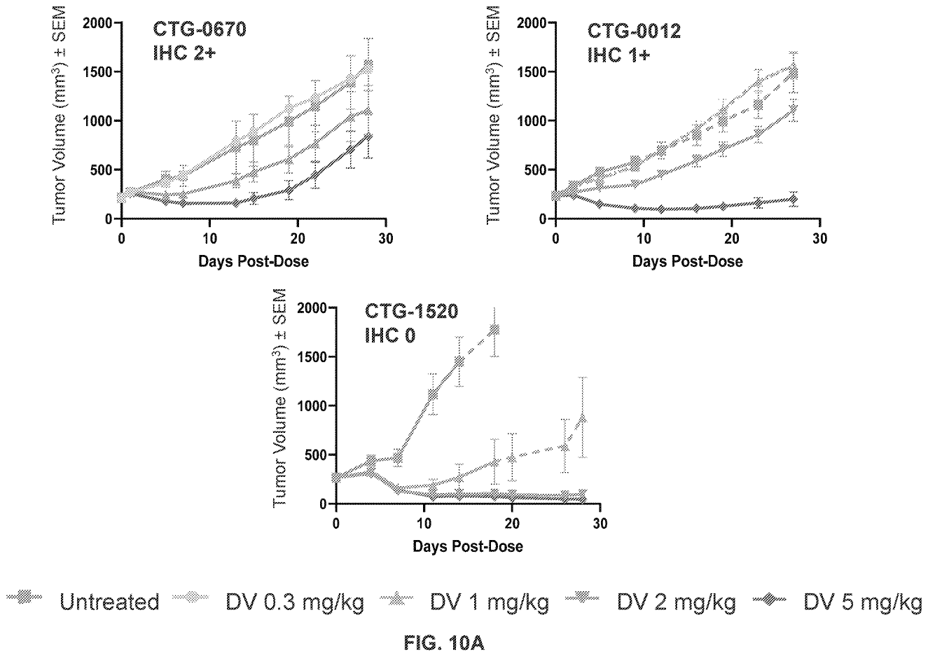

This example describes a phase 1b/2 open label, multicenter study that focuses on subjects with LA/mGC/GEJC and LA/mBC that express HER2. This study has 3 phases: Dose escalation phase evaluating disitamab vedotin plus tucatinib; optimization phase for safety and efficacy using the 2 disitamab vedotin dose levels identified in the dose escalation phase; expansion phase with 2 cohorts based on tumor type and HER2 expression level (FIG. 9).

The dose escalation phase determines the safety and tolerability of disitamab vedotin at various dose levels plus tucatinib in subjects with HER2-expressing (TIC 1+ or higher) 2L LA/mGC/GEJC, or 3L or higher LA/mBC. The tucatinib dose is fixed at 300 mg orally (PO) twice daily (BID) starting Cycle 1 Day 8. The disitamab vedotin starting dose level (level 1) is 1.25 mg/kg. Dose level can escalate or de-escalate based on dose limiting toxicity (DLT) evaluation by the safety monitoring committee (SMC). Intra-patient dose escalation is not permitted.

The optimization phase evaluates disitamab vedotin monotherapy and 2 dose levels of disitamab vedotin plus tucatinib (300 mg PO BID starting on Cycle 1 Day 1) in HER2-low LA/mBC subjects in a randomized fashion (Cohort A). In the event only 1 dose level of disitamab vedotin in combination with tucatinib is determined to be suitable, one of the disitamab vedotin plus tucatinib treatment arms in Cohort A may not be opened and the expansion and optimization phases (Cohorts A, B and C) are accrued simultaneously. A benefit-risk assessment taking into account overall safety, pharmacokinetics (PK) and efficacy data guides the identification of the optimal disitamab vedotin dose in combination with tucatinib to be used in the expansion phase. The optimal disitamab vedotin plus tucatinib combination dose selected for the expansion phase may also include modifications to the tucatinib dose. Subjects in Cohort A are stratified by local HER2 status (IHC 1+vs. IHC 2+/ISH negative).
Cohort A (HER2-low LA/mBC):
   Arm 1: disitamab vedotin monotherapy
   Arm 2: disitamab vedotin (recommended dose number 1) plus tucatinib
   Arm 3: disitamab vedotin (recommended dose number 2) plus tucatinib

After the optimization phase is completed, enrollment in Cohorts B and C for the expansion phase proceeds as follows:
Cohort B (HER2+LA/mBC)
   Arm 1: disitamab vedotin monotherapy
   Arm 2: disitamab vedotin plus tucatinib
Cohort C (HER2-low LA/mGC/GEJC):
   Arm 1: disitamab vedotin monotherapy
   Arm 2: disitamab vedotin plus tucatinib

Subjects are randomized into each treatment arm following screening. In Cohort B, subjects are stratified by the presence or absence of visceral metastases. In Cohort C, subjects are stratified by local HER2 status (IHC 1+vs. IHC 2+/in situ hybridization [ISH]-negative).

The overall sample size of the study is up to 198 subjects with approximately 6 to 18 subjects enrolled in the dose escalation phase, and approximately 60 subjects in each of Cohorts A, B, and C for the optimization and expansion phases.

Cohort A randomizes approximately 60 subjects across 3 planned treatment arms (20 subjects in each treatment arm). In the event only 1 dose level of disitamab vedotin in combination with tucatinib is evaluated in Cohort A, approximately 60 subjects are accrued across 2 treatment arms.

Cohorts B and C randomize approximately 60 subjects across 2 planned treatment arms (30 subjects in each treatment arm).

### Treatment

A schema for the study is shown in FIG. 9.

For the dose escalation phase, screening of subjects occurs within 28 days prior to Cycle 1 Day 1. The safety and tolerability of the disitamab vedotin plus tucatinib combination are determined in the dose escalation phase. Subjects with HER2-expressing (IHC 1+ or higher) 3L+LA/mBC or 2L LA/mGC/GEJC are eligible. Approximately 18 subjects are enrolled in the dose escalation phase and receive disitamab vedotin at various dose levels in combination with tucatinib. The starting dose level for disitamab vedotin is 1.25 mg/kg on Day 1 of each 2-week cycle. Tucatinib 300 mg is given PO BID starting on Cycle 1 Day 8 to allow the assessment of any potential impact of tucatinib on the PK of monomethyl auristatin E (MMAE).

The mTPI is used to evaluate safety and tolerability of the disitamab vedotin plus tucatinib combination. The mTPI dose-escalation method has the ability to estimate the maximum (MTD) with high accuracy and treatment of fewer subjects above the MTD, thereby improving safety and allowing for flexible cohort sizes. Additionally, the mTPI uses information from all subjects treated at all dose levels for estimation of the MTD to improve accuracy of estimation. If the MTD is not reached, a safe and biologically effective recommended dose may be determined via real-time assessment of safety, available activity, PK, immunogenicity, and pharmacodynamic data from across all evaluated doses. A minimum of 6 DLT-evaluable subjects, among whom the DLT rate is <30%, are observed at the estimated MTD or recommended optimization doses before the final MTD or recommended expansion dose is determined. Dose de-escalation may be performed at any time by the sponsor in consultation with the SMC.

Disitamab vedotin is administered at various dose levels on Day 1 of each 2-week cycle starting on Day 1 of Cycle 1. Tucatinib is administered 300 mg PO BID starting on Day 8 of Cycle 1. DV dose levels are 1.0 mg/kg (dose level −1), 1.25 mg/kg (starting dose), and 1.5 mg/kg (dose level +1). Dosing of disitamab vedotin is based on the subject's actual body weight. Doses must be adjusted for subjects who experience a ≥10% change in weight from baseline. Subject weight must be measured during all relevant assessment windows. Other dose adjustments for changes in body weight are permitted per institutional standard. The maximum weight for disitamab vedotin dose calculation is 100 kg. For subjects weighing more than 100 kg, total dose is calculated using 100 kg. In the event of a dose reduction, the weight used to calculate the total dose is 100 kg for subjects weighing more than 100 kg. Disitamab vedotin infusion duration is around 60 minutes (range from 30 to 90 minutes). Infusion time can be longer if needed. The minimum time between doses of disitamab vedotin is 11 days.

DLTs are evaluated in DLT-evaluable subjects receiving disitamab vedotin plus tucatinib in the dose escalation phase. The DLT evaluation period is the first 28 days starting Cycle 1 Day 1 (Study Day 1 through Study Day 28). A subject is considered DLT evaluable when a subject:
Completes the DLT-evaluation period and receives ≥75% of intended disitamab vedotin and tucatinib doses.
Experiences a DLT during the DLT-evaluation period

A DLT is defined as any of the following during the DLT-evaluation period if assessed by the investigator to be clinically significant and related to disitamab vedotin or tucatinib treatment. Grading is according to NCI CTCAE v5.0:
Grade 5 toxicity unrelated to underlying disease or extraneous causes
Any treatment-related toxicity that leads to permanent discontinuation of either or both study drug(s)
Hematologic toxicity:
   Grade 3 or 4 febrile neutropenia
   Grade 4 hematologic toxicity lasting ≥7 days, except thrombocytopenia
   Grade 4 thrombocytopenia of any duration
   Grade 3 thrombocytopenia associated with significant bleeding
Grade 3 or higher hyperglycemia
Grade 3 or higher electrolyte abnormalities associated with symptoms of any duration. Asymptomatic Grade 3 or higher electrolyte abnormalities are not considered to be a DLT.
Grade 3 or higher rash
Grade 3 or higher arthralgia
Any Grade 4, and Grade 3 fever that does not resolve within 24 hours despite optimal management
IRRs:
   Any Grade 4 IRR
   Grade 3 IRR that does not resolve to ≤Grade 2 within 24 hours with infusion interruption, infusion rate reduction, and/or standard supportive measures
   Grade 3 or higher IRR in subjects receiving premedication
Any nonhematologic toxicity ≥Grade 3 in severity is considered a DLT, except:
   Grade 3 fatigue, or constipation lasting ≤3 days
   Grade 3 diarrhea, nausea, vomiting that resolves to ≤Grade 1 with standard intervention in ≤3 days
Hepatic: any of the following not due to disease progression or intercurrent illness:
   Grade 3 or 4 AST or ALT elevation
   Grade 3 or 4 bilirubin elevation (regardless of transaminases)
   Any instance of AST/ALT elevations >3.0×upper limit of normal (ULN) AND total bilirubin elevation >2.0×ULN

Subjects may continue treatment with the study drugs until progressive disease (PD) is determined per RECIST v1.1 by investigator, unacceptable toxicity, withdrawal of consent, or study closure, whichever occurs first. If a study treatment(s) is discontinued for reasons other than disease progression, the subject continues radiologic tumor assessment until disease progression is determined by the investigator according to RECIST v1.1. Subjects who discontinue from study treatment(s) remain on study for follow-up unless they withdraw consent.

For the optimization phase, screening of subjects occurs within 28 days prior to randomization. The optimization phase evaluates disitamab vedotin monotherapy and 2 dose levels of disitamab vedotin plus tucatinib in HER2-low LA/mBC subjects in a randomized fashion (Cohort A). Subjects in Cohort A are stratified by local HER2 status (IHC 1+vs. IHC 2+/ISH-negative). Subjects are randomized to 1 of 3 treatment arms:
Arm 1: disitamab vedotin monotherapy administered via IV at 1.5 mg/kg on Day 1 of each 2-week cycle starting on Cycle 1 Day 1
Arm 2: disitamab vedotin recommended dose number 1 administered via IV on Day 1 of each 2-week cycle with tucatinib 300 mg PO BID, both starting on Cycle 1 Day 1
Arm 3: disitamab vedotin recommended dose number 2 administered via IV on Day 1 of each 2-week cycle with tucatinib 300 mg PO BID, both starting on Cycle 1 Day 1

For the expansion phase, screening of subjects occurs within 28 days prior to randomization. Subjects enrolled in each cohort are randomized equally to different treatment arms. Treatment arms for Cohort B (HER2+LA/mBC) and Cohort C (HER2-low LA/mGC/GEJC) will be as follows:
Arm 1: disitamab vedotin monotherapy administered IV at 1.5 mg/kg on Day 1 of each 2-week cycle starting on Cycle 1 Day 1
Arm 2: disitamab vedotin administered IV on Day 1 of each 2-week cycle with tucatinib 300 mg PO BID, both starting on Cycle 1 Day 1

The disitamab vedotin dose used in Cohorts B and C is determined in the optimization phase. Subjects may continue treatment with disitamab vedotin and tucatinib in their assigned treatment arm until PD determined per RECIST v1.1 by investigator, unacceptable toxicity, withdrawal of consent, or study closure, whichever occurs first. If a study treatment(s) is discontinued for reasons other than disease progression, the subject continues radiologic tumor assessment until disease progression is determined by the investigator according to RECIST v1.1. Subjects who discontinue from study treatment(s) remain on study for follow-up unless they withdraw consent.

### Objectives and Endpoints

One primary objective is to identify the maximum tolerated dose (MTD) and/or optimal dose of disitamab vedotin when administered in combination with tucatinib. Another primary objective is to characterize the safety and tolerability profile of disitamab vedotin monotherapy regimens in:
HER2-low second-line advanced gastric and gastroesophageal junction adenocarcinoma
HER2+ third-line or higher advanced breast cancer
HER2-low second- or third-line advanced breast cancer

Another primary objective is to characterize the safety and tolerability of disitamab vedotin plus tucatinib in:
HER2-low second-line advanced gastric and gastroesophageal junction adenocarcinoma
HER2+ third-line or higher advanced breast cancer
HER2-low second- or third-line advanced breast cancer

Endpoints for these objectives include:
Incidence of dose-limiting toxicities (DLTs) in dose escalation phase (up to 28 days)
Type, incidence, severity, seriousness, and relatedness of adverse events (AEs) (through 30 days after the last study treatment; ~5 years)
Type, incidence, and severity of laboratory abnormalities as well as significant changes from baseline (through 30-37 days after the last study treatment; ~5 years)
Frequency of treatment interruptions, dose reductions and treatment discontinuations due to AEs
Number of participants with dose alterations (through 30-37 days after the last study treatment; ~5 years)

Another primary objective is to assess the antitumor activity of disitamab vedotin monotherapy in:
HER2-low second-line advanced gastric and gastroesophageal junction adenocarcinoma
HER2+ third-line or higher advanced breast cancer
HER2-low second- or third-line advanced breast cancer

Another primary objective is to assess the antitumor activity of disitamab vedotin plus tucatinib in:
HER2-low second-line advanced gastric and gastroesophageal junction adenocarcinoma
HER2+ third-line or higher advanced breast cancer
HER2-low second- or third-line advanced breast cancer

Endpoints for these objectives include ORR (confirmed CR and confirmed PR) per Response Evaluation Criteria in Solid Tumors version 1.1 (RECIST v1.1) by investigator assessment. This is assessed for approximately 3 years after study treatment.

Secondary objectives include assessing the antitumor activity of both disitamab vedotin monotherapy and disitamab vedotin plus tucatinib. Endpoints for these objectives include: DOR per RECIST v1.1 by investigator assessment (approximately 5 years after study treatment); Disease control rate (DCR) (confirmed CR, confirmed PR, and stable disease) per RECIST v1.1 by investigator assessment (approximately 5 years after study treatment); Progression free survival (PFS) per RECIST v1.1 by investigator assessment (approximately 5 years after study treatment); and OS (approximately 5 years after study treatment).

Another secondary objective is to characterize the pharmacokinetics (PK) of disitamab vedotin, total antibody, and unconjugated MMAE in monotherapy and plus tucatinib, e.g., maximum concentration (Cmax; through 30-37 days after the last study treatment; ~5 years) and/or area under concentration-time curve to time of the last quantifiable concentration (AUClast; approx. 1 month). The endpoints for this objective are estimates of selected PK parameters of disitamab vedotin, total antibody, and unconjugated MMAE.

Another secondary objective is to characterize the immunogenicity of disitamab vedotin. The endpoints for this objective are incidence of anti-drug antibodies (ADA) against disitamab vedotin (through 30-37 days after the last study treatment; ~5 years).

An exploratory objective is to assess biomarkers in relation to response, toxicity, pharmacodynamic relationship, or resistance to disitamab vedotin. Endpoints for this objective include baseline and on treatment profiling of blood for proteins and somatic mutations, as well as tissue HER2, genomic, and gene expression profiles at baseline and on treatment.

Another exploratory objective for the expansion phase is to characterize the PK of tucatinib. Endpoints for this objective include estimates of selected PK parameters of tucatinib.

Another exploratory objective for the expansion phase is to characterize the impact of treatment with disitamab vedotin alone or disitamab vedotin in combination with tucatinib on patient-reported outcomes (PROs). Endpoints for this objective include:
Actual and change from baseline scores on the European Organization for Research and Treatment of Cancer (EORTC) quality of life Core-30 (EORTC QLQ-C30) questionnaire form (BC and GC/GEJC) and EORTC QLQ-OG25 questionnaire form (GC/GEJC only)
Meaningful within-patient change (MWPC) thresholds estimated consistent with current FDA guidance (PGIS and PGIC) (BC and GC/GEJC) Inclusion exclusion criteria

Subjects are eligible to be included in the study only if all the following criteria are met. General inclusion criteria include:
Age ≥18 years
Measurable disease according to RECIST v1.1
An Eastern Cooperative Oncology Group (ECOG) Performance Status score of 0 or 1
Adequate baseline cardiac parameters:
   Left ventricular ejection fraction (LVEF) ≥50% as assessed by echocardiogram (ECHO) or multigated acquisition scan (MUGA) within 4 weeks prior to enrollment
   Fridericia's corrected QT interval (QTcF)<470 ms
The following baseline laboratory data, laboratory values collected within 7 days of screening are acceptable:
   Hemoglobin (Hb) ≥9 g/dL
   Absolute neutrophil count (ANC) ≥1.5×10⁹/L
   Platelet count ≥100×10⁹/L
   ALT and AST≤2.5× upper limit of normal (ULN) without liver metastases or ≤5×ULN with liver metastases
   Serum total bilirubin ≤1.5×ULN or direct bilirubin ≤ULN for subjects with total bilirubin >1.5×ULN; serum total bilirubin ≤3×ULN for subjects with Gilbert's syndrome
   CrCl≥30 mL/min, as calculated using the Cockcroft-Gault formula or measured from a 24-hour urine collection

For dose escalation phase subjects, inclusion criteria include:
Histologically or cytologically confirmed diagnosis of gastric or gastroesophageal junction adenocarcinoma, or breast carcinoma
Locally-advanced, unresectable, or metastatic stage
Any HER2 status IHC 1+ or higher by most recent local assessment
All subjects must have experienced disease progression on or after standard of care therapies, or are intolerant of standard of care therapies

Subjects in Cohort A (HER2-low breast cancer) must meet the following eligibility criteria:
Histologically or cytologically confirmed diagnosis of breast carcinoma
Locally-advanced, unresectable, or metastatic stage
HER2-low status determined by most recent local assessment (IHC1+ or IHC2+/ISH-negative) based on ASCO and College of American Pathologists (CAP) guidelines for assessment of HER2 in BC for interpretation of HER2 expression and amplification
Prior therapies requirements:
   No more than 3 prior systemic cytotoxic chemotherapy regimens (including ADCs) for LA/mBC. Subjects previously treated with (neo)adjuvant chemotherapy and have disease relapsed within 6 month between completion of such treatment (eg, date of last [neo]adjuvant cytotoxic therapy administration) are considered as one line of cytotoxic therapy for LA/mBC
   Subjects with BRCA mutations must have received a PARP-inhibitor, where available and not medically contraindicated
   Must have progression on or after, or intolerant to, T-DXd therapy, sacituzumab govitecan, or other topoisomerase I inhibitor directed therapy, if available after local standard of care therapy
   HR+ subjects:
      Must be endocrine therapy refractory:
         Progressed on ≥2 lines of endocrine therapy in the LA/m setting AND had received a CDK4/6 inhibitor in the adjuvant or metastatic setting OR
         Progressed on 1 line of endocrine therapy in the LA/m setting AND had a relapse while on adjuvant endocrine therapy after definitive surgery for primary tumor AND had received a CDK4/6 inhibitor in the adjuvant or metastatic setting
   Subjects with HR-negative, HER2-low and PD-L1-positive (CPS 10 or greater) tumors must have received pembrolizumab with chemotherapy if available as local standard of care therapy

Subjects in Cohort B (HER2+ breast cancer) must meet the following eligibility criteria:
Histologically or cytologically confirmed diagnosis breast carcinoma
Locally-advanced, unresectable, or metastatic stage
HER2+ status determined by most recent local assessment (IHC3+ or IHC2+/ISH+) according to ASCO and CAP guidelines for assessment of HER2 in BC
All subjects must have:
   Received prior trastuzumab, pertuzumab and a taxane if available as local standard of care therapy
   Had progression on or after, or intolerant to, T-DXd or other topoisomerase I inhibitor directed therapy
   No more than 3 prior systemic cytotoxic chemotherapy regimens (including ADCs) for LA/mBC

Subjects in Cohort C (HER2-low gastric or gastroesophageal junction adenocarcinoma) must meet the following eligibility criteria:
Histologically or cytologically confirmed diagnosis of gastric or gastroesophageal junction adenocarcinoma
Locally-advanced, unresectable, or metastatic stage
HER2-low status determined by most recent local assessment (IHC1+ or IHC2+/ISH-negative) determined by most recent local assessment based off the ASCO and CAP guidelines for assessment of HER2 in GC for interpretation of HER2 expression and amplification
All subjects must have:
   Received prior systemic therapy with platinum, fluorouracil, or taxane for locally advanced unresectable or metastatic disease
   Experienced progression within 6 months of last dose of (neo)adjuvant cytotoxic chemotherapy is considered as one line of systemic therapy for LA/mGC/GEJC
   Prior anti-PD-(L)1 therapy is allowed
   No more than 2 prior systemic cytotoxic chemotherapy regimens (including ADC) for LA/mGC/GEJC
Subjects must not have received prior treatment with HER2 directed therapy.

General exclusion criteria include:
Known hypersensitivity to any excipient contained in the drug formulation of disitamab vedotin or tucatinib
Prior therapy with ADCs with MMAE payload
Prior therapy with tucatinib
CNS and/or leptomeningeal metastasis
   Subjects with treated CNS metastases (by whole brain radiation therapy, surgery or radiosurgery, etc.) are permitted on study if all of the following are met: CNS metastases have been clinically stable for at least 4 weeks and baseline scans show no evidence of new or worsening CNS metastasis; Subject is on a stable dose of ≤10 mg/day of prednisone or equivalent for at least 2 weeks; and Subject does not have leptomeningeal metastasis
Subjects who have received prior systemic anticancer treatment including investigational agents within 4 weeks (please consult medical monitor for permitting shorter interval for kinase inhibitors or other short half-life anticancer drugs) prior to first dose of study treatment. Participants must have recovered from all AEs due to previous therapies to ≤Grade 1 or baseline (except for alopecia). Participants with ≤Grade 1 neuropathy may be eligible. If the participant had major surgery, the participant must have recovered adequately from the procedure and/or any complications from the surgery prior to starting study intervention.
Subjects with acute, chronic, or symptomatic infections, including: Ongoing symptomatic severe acute respiratory syndrome-associated coronavirus 2 (SARS-CoV-2) infection except for subjects who have recovered clinically but continue to have a detectable presence of SARS-CoV-2; History of human immunodeficiency virus (HIV) infection (testing is not required unless mandated by local regulations); Hepatitis B virus (HBV) infection (defined as positive for HBV surface antigen with positive HBV DNA) or known active hepatitis C virus (HCV) infection (defined as HCV RNA [qualitative] is detected). Subjects who have been curatively treated for hepatitis C infection are permitted if they have documented sustained virologic response of ≥12 weeks. HBV negative DNA of ≥12 weeks is also allowed. No HBV or HCV testing is required, unless mandated by local regulations or institutional standard.
Subjects with a history of another invasive malignancy within 3 years before the first dose of study intervention, or any evidence of residual disease from a previously diagnosed malignancy
   Subjects with adequately resected early-stage non-melanoma skin cancer or carcinoma in situ are allowed
   Subjects with a history of prostate cancer (T2NXMX or lower with Gleason score ≤7) treated with definitive intent (surgically or with radiation therapy) is acceptable, provided that the subject is considered prostate cancer free and the following criteria are met: Subjects who have undergone radical prostatectomy must have undetectable prostate-specific antigen (PSA) for >1 year and at screening; Subjects who have had radiation must have a PSA doubling time >1 year (based on at least 3 values determined >1 month apart) and a total PSA value that does not meet Phoenix criteria for biochemical recurrence (ie, <2.0 ng/mL above nadir); and Subjects with untreated low-risk prostate cancer (Gleason score ≤6) on active surveillance with PSA doubling time >1 year (based on at least 3 values determined >1 month apart) are also eligible
   Subjects with bone-only metastatic lesions are excluded
Uncontrolled cardiac disease including:
   Cardiac failure—New York Heart Association (NYHA) Class III or IV heart failure
   Cardiac arrhythmia—Grade 2 or higher arrhythmia or heart block
   Cardiac ischemia—unstable angina within the past 12 months, myocardial infarction or cerebral infarction within the past 6 months, etc.
   Hypertension—uncontrolled hypertension (systolic blood pressure >180 mmHg and/or diastolic blood pressure >100 mmHg)
Subjects who have received radiotherapy within 2 weeks. Subject must have recovered adequately from all radiation-related toxicities. A 1-week washout is permitted for palliative radiation (≤2 weeks duration of radiotherapy) to non-CNS disease.
Subjects who have received major surgery within 4 weeks prior to Cycle 1 Day 1 (dose escalation phase) or randomization in the optimization or expansion phases must have recovered adequately
Subjects requiring chronic oxygen therapy or have ≥Grade 3 dyspnea, hypoxia, or other pulmonary disease unrelated to underlying malignancy
Subjects who have received a live or live-attenuated vaccine within 30 days prior to Cycle 1 Day 1 (dose escalation phase) or randomization in the optimization or expansion phases. Administration of killed vaccines is allowed
Subjects who have received whole blood or plasma transfusions within 14 days prior to Cycle 1 Day 1 (dose escalation phase) or randomization in the optimization or expansion phases
Other serious underlying medical condition that, in the opinion of the investigator, would impair the subject's ability to receive or tolerate the planned treatment and follow-up
Have used a strong cytochrome P450 (CYP)2C8 inhibitor within 5 half-lives of the inhibitor or have used a strong CYP2C8 or CYP3A4 inducer within 5 days prior to first dose of study treatment
Bowel obstruction, history or presence of inflammatory enteropathy or extensive intestinal resection (hemicolectomy or extensive small intestine resection with chronic diarrhea) or moderate to severe chronic diarrhea or any conditions may alter oral drug absorption.
Presence of known active/chronic liver disease
Has ongoing ≥Grade 2 diarrhea of any etiology at screening
Pleural effusion or ascites with symptoms or requiring symptomatic treatment
Unable to swallow oral tablets or capsules or any significant GI disease which would preclude the adequate oral absorption of medications

### Example 5: Anti-Tumor Effects of DV+Tucatinib in HER2-Low Breast Cancer Xenograft Models

This Example describes studying the effects of DV+tucatinib vs. either agent alone in HER2-low breast cancer patient-derived xenograft (PDX) models.

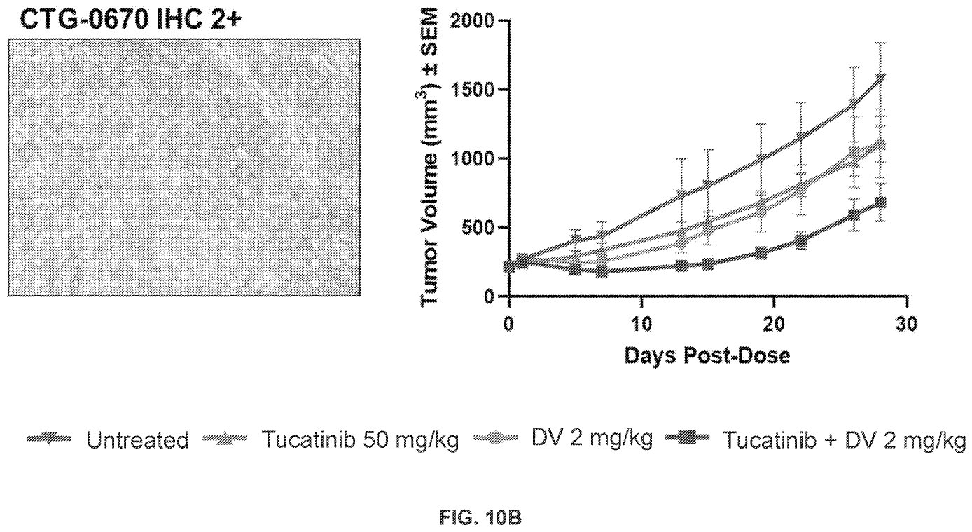

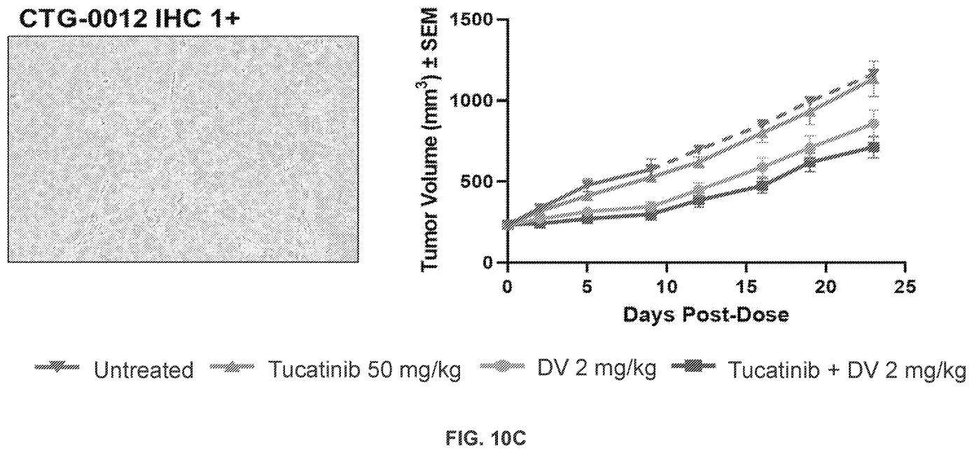

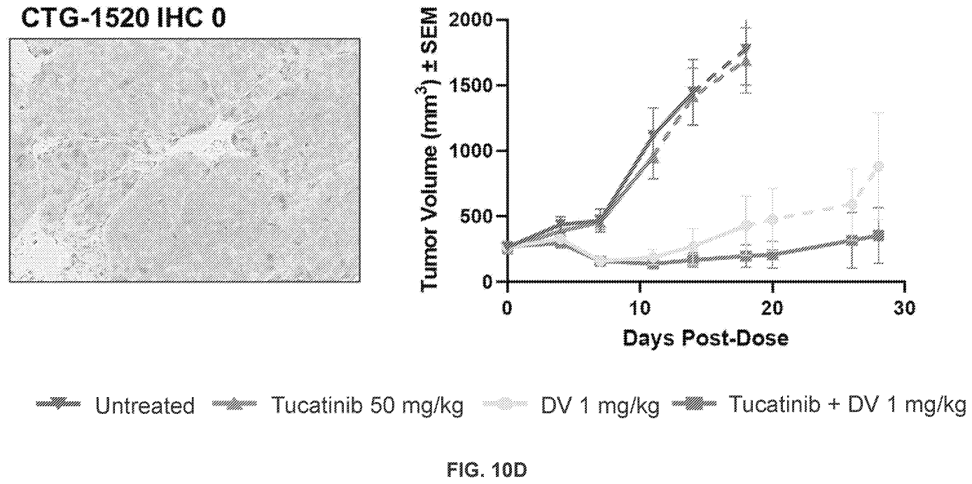

First, the anti-tumor effect of DV as a monotherapy was tested in three PDX models with varying HER2 expression levels. Three breast cancer PDX models were used: CTG-1520 (IHC 0), CTG-0012 (IHC 1+), and CTG-0670 (IHC 2+). IHC images for each model are shown in FIGS. 10B-10D. Mice (n=10/group) were treated with the indicated concentrations of DV (intravenous, single dose), and tumor volume was measured over time. The results demonstrated that a single dose of DV showed anti-tumor effects in a dose-dependent manner in a subset of HER2-low breast cancer xenograft models (FIG. 10A). Dashed lines represent carry-over of tumor volumes from individual animals that were removed from study when their tumors reached maximum allowable size (less than 3/10 animals per group).

Next, DV and tucatinib were tested as monotherapies and in combination in the three PDX models. Mice (n=10/group) were treated with the indicated concentrations of tucatinib (50 mg/kg oral, twice daily), DV (1 or 2 mg/kg, intravenous, single dose), or a combination of tucatinib and DV, and tumor volume was measured over time. At sub-maximal doses of DV (1.0-2.0 mg/kg), the combination of DV and tucatinib improved tumor growth inhibition in 2 out of 3 models (FIGS. 10B-10D). Dotted lines represent carry-over of tumor volumes from individual animals that were removed from study when their tumors reached maximum allowable size (less than 3/10 animals per group). These results demonstrate that the combination of DV+ tucatinib showed improved anti-tumor activity with sub-therapeutic doses of DV in a subset of HER2-low breast cancer xenograft models.

### Example 6: Cytotoxicity of DV in 2D Breast Cancer Cell Lines and Patient-Derived Tumor Organoids with Varying HER2 Expression Levels

This Example describes the in vitro cytotoxicity of DV as compared to trastuzumab deruxtecan (T-DXd) in 2D breast cancer cell lines and in patient-derived organoids (PDOs).

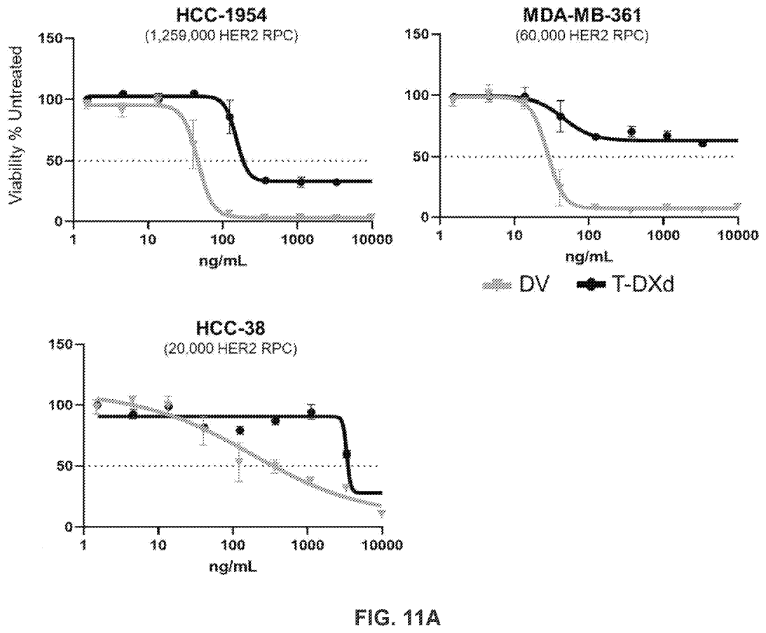

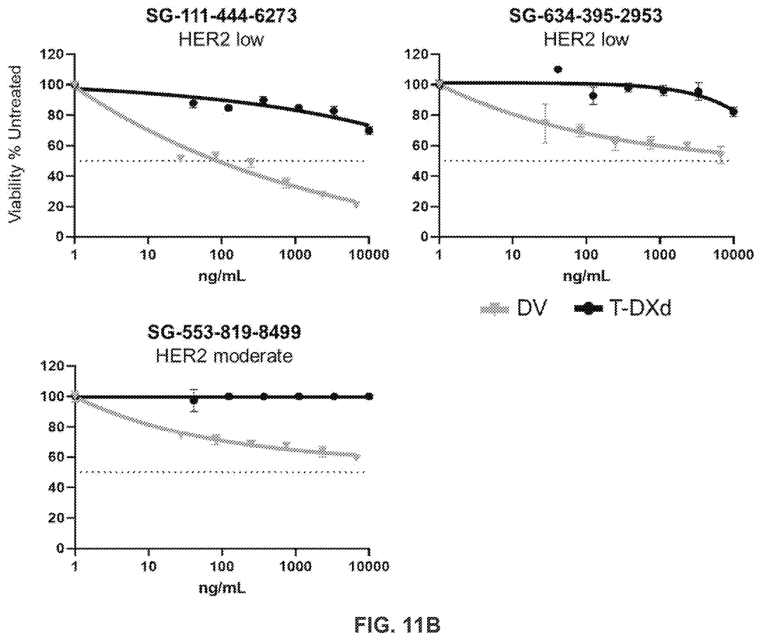

DV and T-DXd were evaluated against breast cancer cell lines spanning a range of HER2 expression levels (calculated by quantitative flow cytometry): HCC-1954 with 1,259,000 HER2 receptors/cell, MDA-MB-361 with 60,000 HER2 receptors/cell, and HCC-38 with 20,000 HER2 receptors/cell. Drugs were titrated (1-10,000 ng/mL), and viability was tested in a 144-hour CellTiter-Glo cytotoxicity assay. The results showed more potent single-agent cytotoxic activity for DV as compared to T-DXd, and DV's anti-tumor effects were dose-dependent (FIG. 11A).

DV and T-DXd were also tested against a panel of PDOs: HER2 low SG-111-444-6273, HER2 low SG-634-395-2953, and HER2 moderate SG-553-819-8499. DV was titrated across a range of 27-6,700 ng/mL, while T-DXd was titrated across a range of 41-10,000 ng/mL. Drug and media was exchanged every four days. Analysis was conducted with fluorescence imaging: PDOs were identified from clusters of Hoechst positive cells, and caspase-3/7 was used for the identification of apoptotic cells. Mean caspase-3/7 intensity was measured per well and normalized to cell count. The results showed more potent single-agent cytotoxic activity for DV as compared to T-DXd, and DV's anti-tumor effects were dose-dependent (FIG. 11B).

### Example 7: Antitumor Effects of DV+Tucatinib in Gastric Cancer Xenograft Models with Variable HER2 Expression Levels

This Example describes studying the effects of DV+tucatinib vs. DV alone in gastric cancer PDX models with variable HER2 expression levels.

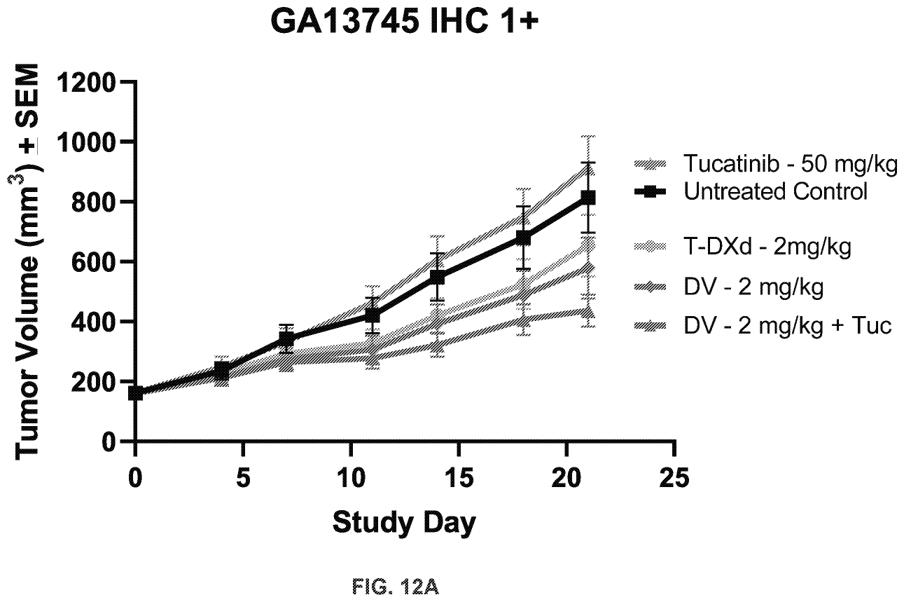

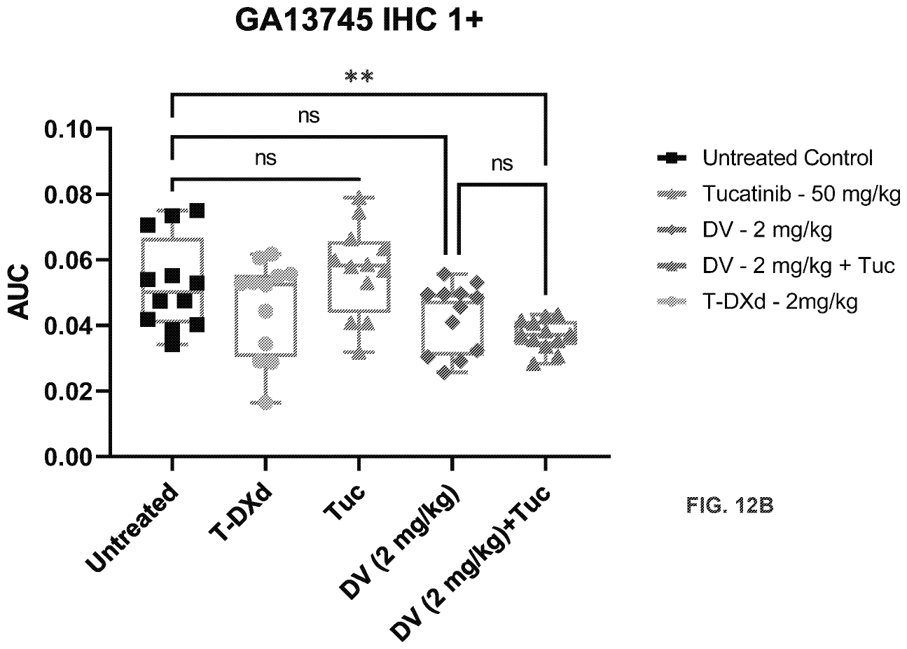

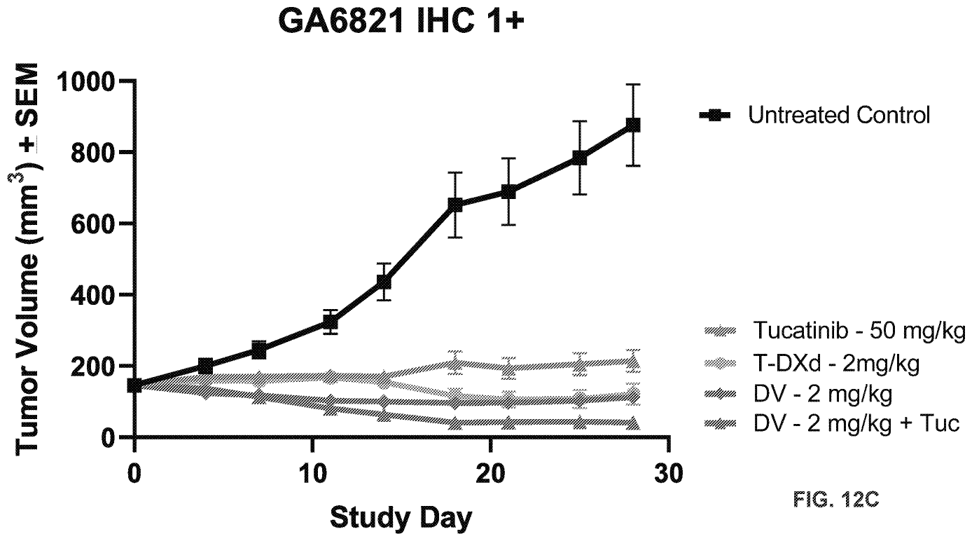

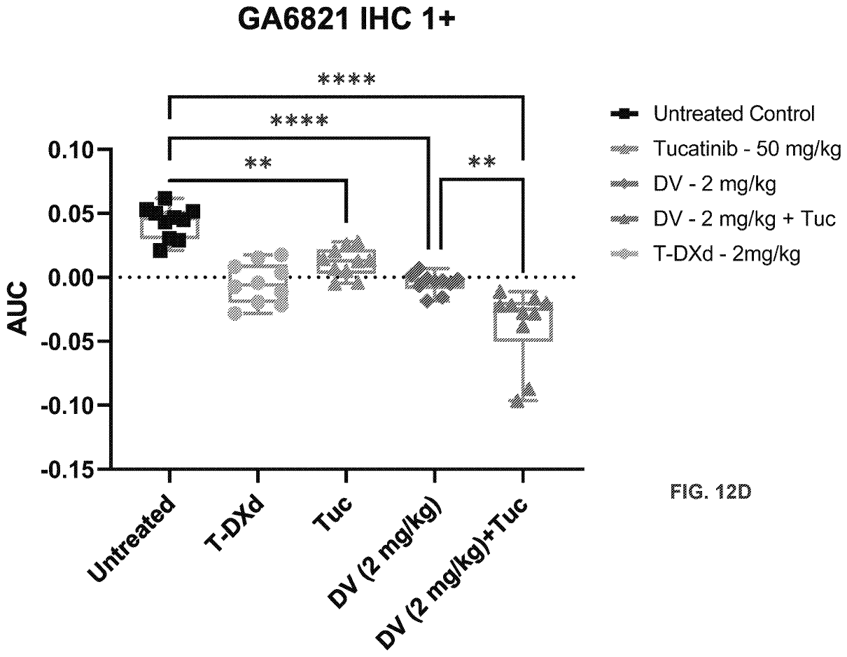

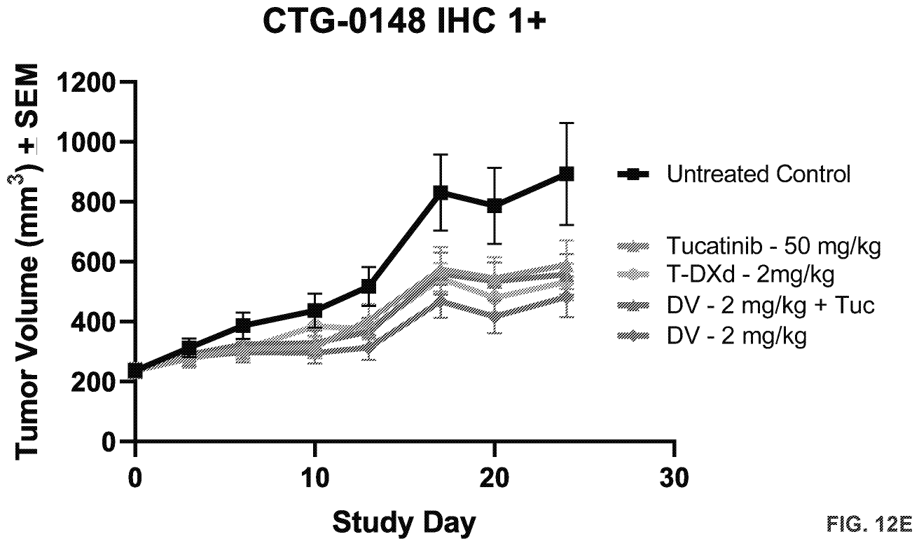

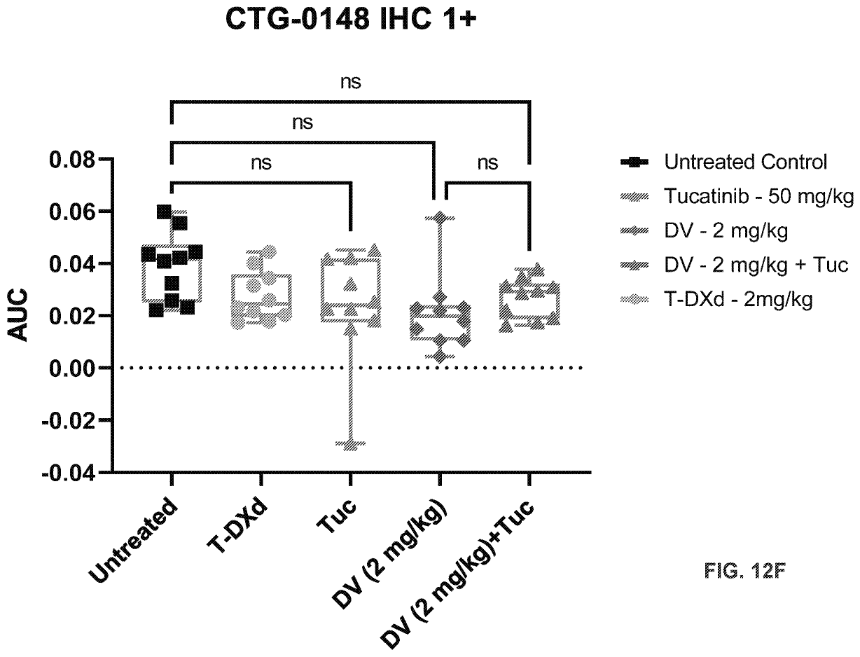

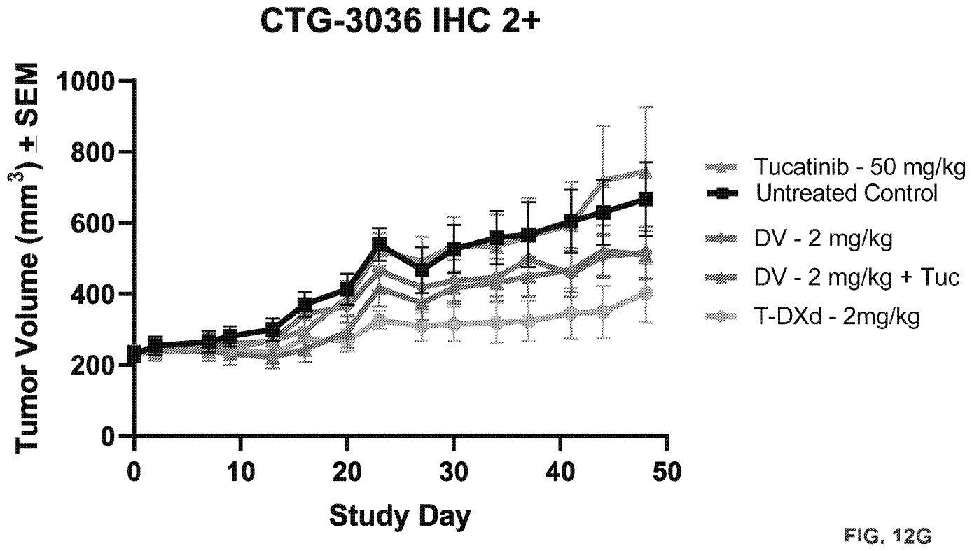

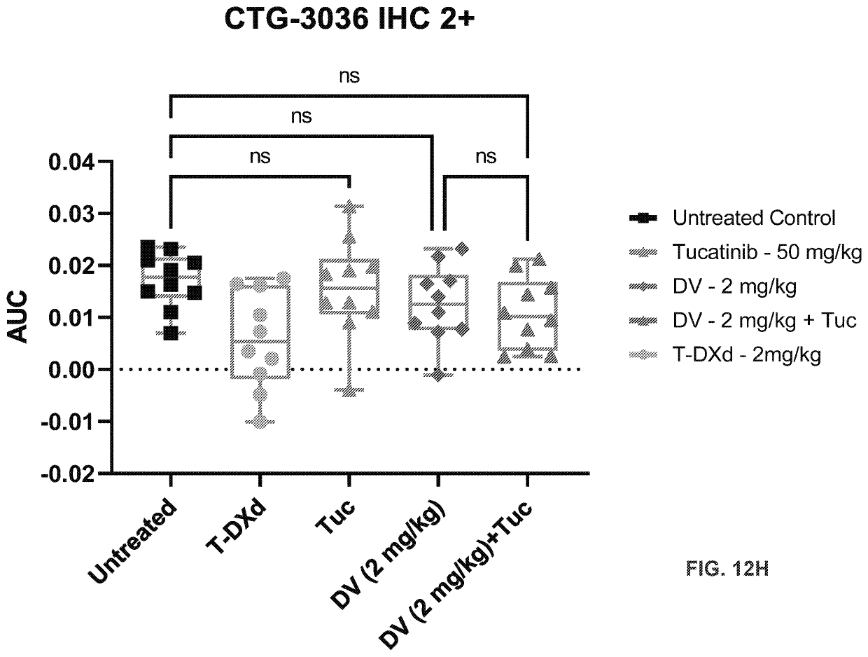

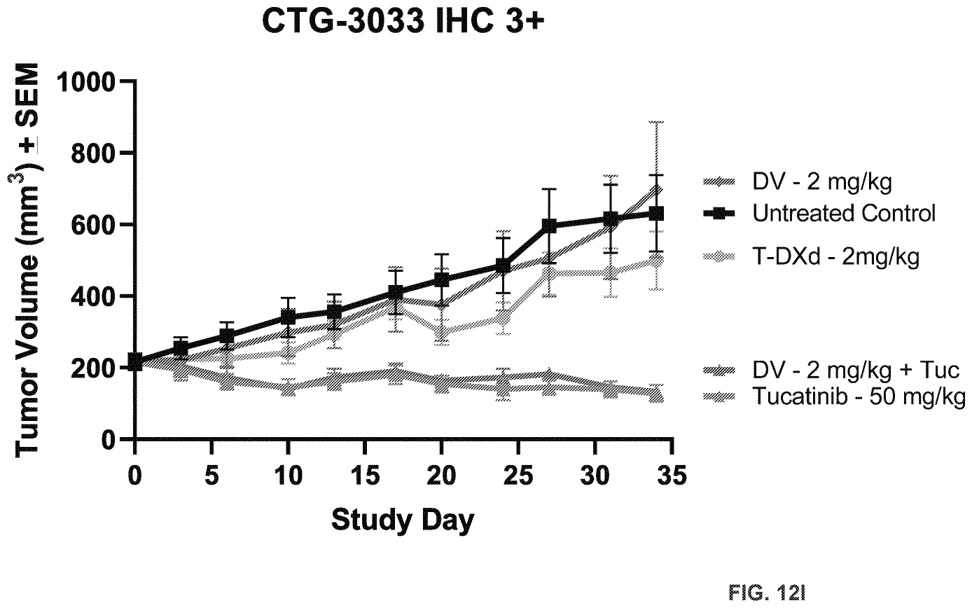

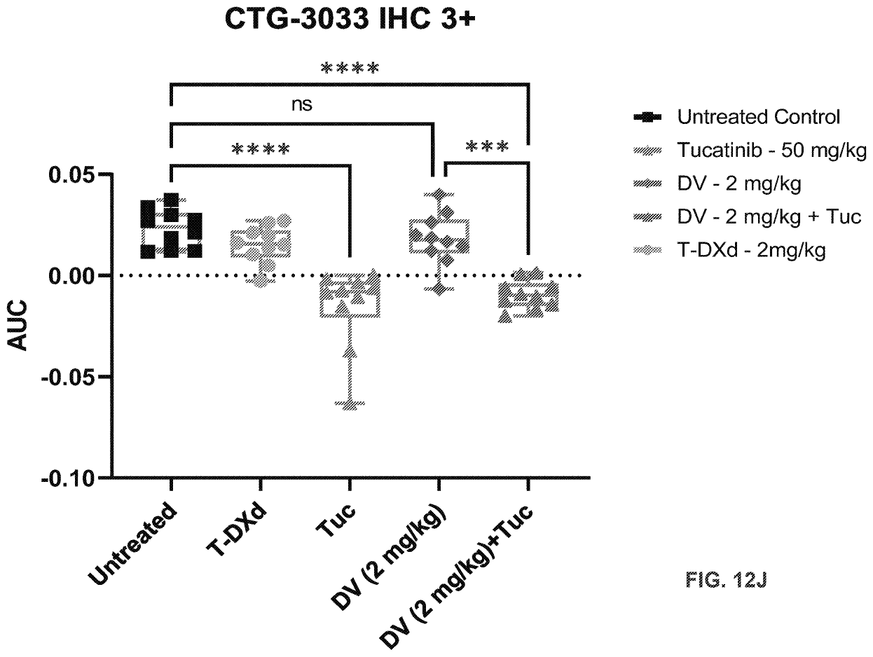

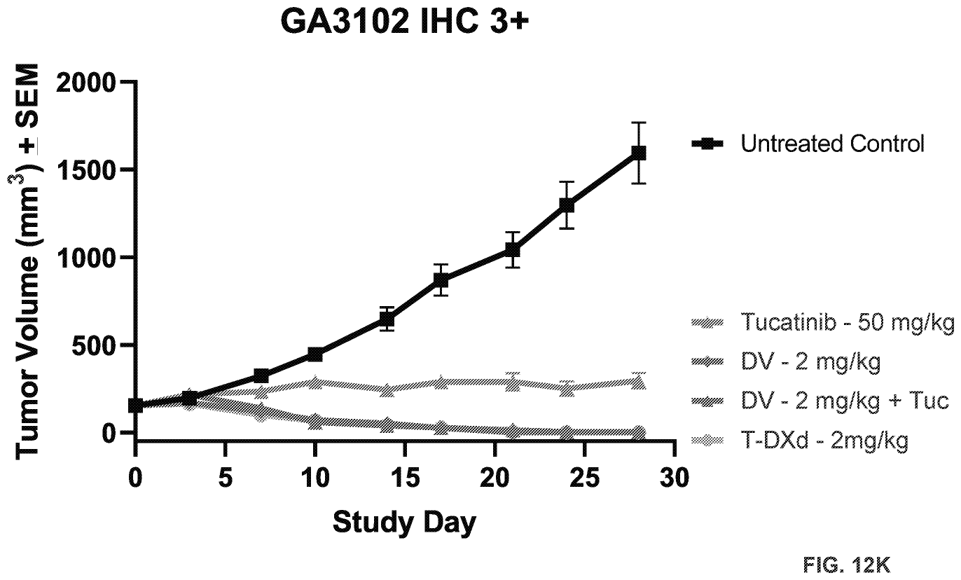

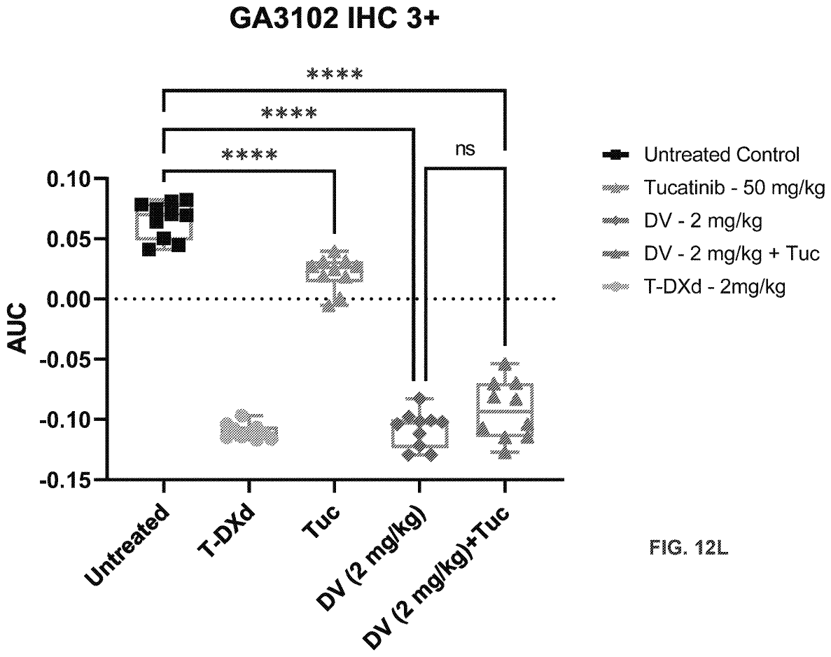

DV and tucatinib were tested as monotherapies and in combination in PDX models with varying HER2 expression levels. Six gastric cancer PDX models were used: GA13745 (IHC 1+), GA6821 (IHC 1+), CTG-0148 (IHC 1+), CTG-3036 (IHC 2+), CTG-3033 (IHC 3+), and GA3102 (IHC 3+). Mice (n=10/group) were treated with tucatinib (50 mg/kg oral, twice daily), DV (2 mg/kg, intravenous, single dose), a combination of tucatinib and DV, or T-DXd (2 mg/kg, intravenous, single dose), and tumor volume was measured over time. In a majority of the PDX models tested, DV showed anti-tumor activity, and in particular exhibited significant anti-tumor activity in two of the models (FIGS. 12C, 12K), as demonstrated by the statistically significant difference in the normalized area under the curve (AUC) tumor growth metric between the untreated group and DV monotherapy group (FIGS. 12D and 12L). Additionally, in one of these models, the combination of DV+tucatinib showed improved anti-tumor activity compared to DV alone (FIG. 12D). In a majority of the PDX models tested (FIGS. 12A, 12C, 12I, 12K), the combination of DV+tucatinib showed significant anti-tumor activity compared to untreated control (FIGS. 12B, 12D, 12J, 12L), including in one model where no significant single agent activity was observed (FIG. 12B). These results demonstrate the robust anti-tumor activity of DV as monotherapy and in combination with tucatinib. Taken together, these results show that DV as monotherapy and in combination with tucatinib has anti-tumor activity across a diverse subset of PDX gastric cancer models, with variable HER2 expression levels that reflect the gastric cancer patient population.

What is claimed is:

1. A method for treating or preventing progression of cancer in an individual, comprising administering to the individual an effective amount of tucatinib and an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); and wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

2. A method for treating or preventing progression of cancer in an individual, comprising administering to the individual an effective amount of tucatinib and an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:11); and wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

3. The method of claim 1 or claim 2, wherein the VH domain comprises the amino acid sequence of SEQ ID NO:7.

4. The method of any one of claims 1-3, wherein the VL domain comprises the amino acid sequence of SEQ ID NO:8.

5. The method of any one of claims 1-4, wherein the heavy chain comprises the amino acid sequence of SEQ ID NO:9.

6. The method of any one of claims 1-5, wherein the light chain comprises the amino acid sequence of SEQ ID NO:10.

7. The method of any one of claims 1-6, wherein the cytotoxic molecule comprises a tubulin inhibitor or DNA damaging agent.

8. The method of claim 7, wherein the tubulin inhibitor comprises a dolastatin or derivative thereof, auristatin or derivative thereof, or maytansinoid or derivative thereof.

9. The method of claim 8, wherein the tubulin inhibitor comprises monomethyl auristatin E (MMAE), monomethyl auristatin F (MMAF), or auristatin F (AF).

10. The method of claim 8, wherein the tubulin inhibitor comprises emtansine (DM1), maytansine (DM3), or ravtansine (DM4).

11. The method of claim 7, wherein the DNA damaging agent comprises a calicheamicin, duocarmycin, pyrrolobenzodiazepine (PBD), or SN-38.

12. The method of any one of claims 1-6, wherein the cytotoxic molecule comprises an amanitin, anthracycline, baccatin, camptothecin, cemadotin, colchicine, colcimid, combretastatin, cryptophycin, dicodermolide, docetaxel, doxorubicin, echinomycin, eleutherobin, epothilone, estramustine, lexitropsin, maytansine, methotrexate, netropsin, puromycin, rhizoxins, taxane, tubulysin, or vinca alkaloid.

13. The method of any one of claims 1-12, wherein the antibody-drug conjugate is represented by formula Ab-(L-U)n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody.

14. The method of claim 13, wherein the linker is attached to the anti-HER2 antibody via a thiol or amino moiety.

15. The method of claim 13 or claim 14, wherein the linker is selected from the group consisting of maleimidocaproyl valine citrulline p-amino-benzyloxy (mc-vc-pAB), maleimidocaproyl (mc), triglycyl peptide linker, 3-maleimido-propionic acid, Mal-di-EG-OPFP (perfluorophenyl 3-(2-(2-(3-(2,5-dioxo-2,5-dihydro-1H-pyrrol-1-yl)propanamido)ethoxy)ethoxy)propanoate), Mal-di-EG-Osu (2,5-dioxopyrrolidin-1-yl 3-(2-(2-(2,5-dioxo-2,5-dihydro-1H-pyrrol-1-yl)ethoxy)ethoxy)propanoate), Mal-Tri-EG-Osu (2,5-dioxopyrrolidin-1-yl 3-(2-(2-(2-(2,5-dioxo-2,5-dihydro-1H-pyrrol-1-yl)ethoxy)ethoxy)ethoxy)ethoxypropanoate), Mal-Tetra-EG-Osu (2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate), Br-di-EG-Osu (2,5-dioxopyrrolidin-1-yl 3 (2-(2-(2-bromoacetamido)ethoxy)ethoxy)propanoate), Py-ds-prp-Osu (2-5-dioxopyrrolidin-1-yl 3-(pyridine-2-yldisulfanyl)propanoate), Py-ds-Prp-OPEP (perfluorophenyl 3-(pyridine-2-yldisulfanyl)propanoate), Py-ds-dmBut-Osu (2,5-dioxopyrrolidin-1-yl 4-methyl-4-(pyridine-2-yldisulfanyl)pentanoate, Py-ds-dmBut-OPF (perfluorophenyl 4-methyl-4-(pyridine-2-yldisulfanyl)pentanoate), SMCC (N-succinimidyl 4-(maleimidomethyl) cyclohexanecarboxylate), MBS (3-maleimidobenzoic acid N-hydroxysuccinimide ester), SATA (S—(N-succinimidyl)thioacetate), SPDP ((N-succinimidyl 3-(2-pyridyldithio)propionate), and SMPT ((N-succinimidyloxy carbonyl)-1-methyl-1-(2-pyridyldithio)toluene).

16. The method of claim 1 or 2, wherein the antibody-drug conjugate is disitamab vedotin.

17. The method of any one of claims 1-16, wherein the cancer has previously been treated.

18. The method of any one of claims 1-17, wherein:
(a) prior to administration of tucatinib and the antibody-drug conjugate, the individual has experienced progression on or after one or more standard of care therapies; or
(b) the individual is intolerant to one or more standard of care therapies.

19. The method of any one of claims 1-18, wherein cells of the cancer express HER2.

20. The method of any one of claims 1-19, wherein the cancer is breast cancer.

21. The method of claim 20, wherein the cancer is locally advanced or metastatic breast cancer (LA/mBC).

22. The method of claim 20 or claim 21, wherein the administration of tucatinib and the antibody-drug conjugate is a second-line (2L) treatment.

23. The method of claim 2020 or claim 21, wherein the administration of tucatinib and the antibody-drug conjugate is a third-line or higher (3L+) treatment.

24. The method of any one of claims 20-23, wherein the cancer is a HER2-low cancer.

25. The method of claim 24, wherein a sample obtained from the individual comprises cancer cells that express HER2 on their cell surface at a level of IHC1+, as measured by immunohistochemistry (IHC) assay.

26. The method of claim 24, wherein a sample obtained from the individual comprises cancer cells that express HER2 on their cell surface at a level of IHC2+, as measured by IHC assay; and a sample obtained from the individual comprises cancer cells that do not exhibit HER2 gene amplification, as measured by in situ hybridization (ISH) assay (ISH-negative).

27. The method of any one of claims 20-23, wherein the cancer is a HER2-positive cancer.

28. The method of claim 27, wherein a sample obtained from the individual comprises cancer cells that express HER2 on their cell surface at a level of IHC3+, as measured by IHC assay.

29. The method of claim 27, wherein a sample obtained from the individual comprises cancer cells that express HER2 on their cell surface at a level of IHC2+, as measured by IHC assay; and a sample obtained from the individual comprises cancer cells that exhibit HER2 gene amplification, as measured by ISH assay (ISH-positive).

30. The method of claim 20, wherein the cancer is HER2-positive LA/mBC; and wherein the administration of tucatinib and the antibody-drug conjugate is a third-line or higher (3L+) treatment.

31. The method of claim 30, wherein the individual has a visceral organ metastasis.

32. The method of claim 30, wherein the individual does not have a visceral organ metastasis.

33. The method of claim 20, wherein the cancer is HER2-low LA/mBC; and wherein the administration of tucatinib and the antibody-drug conjugate is a second-line (2L) treatment or a third-line or higher (3L+) treatment.

34. The method of claim 33, wherein a sample obtained from the individual comprises cancer cells that express HER2 on their cell surface at a level of IHC1+, as measured by immunohistochemistry (IHC) assay.

35. The method of claim 33, wherein a sample obtained from the individual comprises cancer cells that express HER2 on their cell surface at a level of IHC2+, as measured by IHC assay; and a sample obtained from the individual comprises cancer cells that do not exhibit HER2 gene amplification, as measured by in situ hybridization (ISH) assay (ISH-negative).

36. The method of any one of claims 1-19, wherein the cancer is gastric cancer or gastroesophageal junction cancer (GEJC).

37. The method of claim 36, wherein the cancer is gastric adenocarcinoma or gastroesophageal junction adenocarcinoma.

38. The method of claim 36 or claim 37, wherein the cancer is locally advanced or metastatic gastric cancer or GEJC (LA/mGC/GEJC).

39. The method of any one of claims 36-38, wherein the administration of tucatinib and the antibody-drug conjugate is a second-line (2L) treatment.

40. The method of any one of claims 36-39, wherein the cancer is a HER2-positive cancer.

41. The method of claim 40, wherein a sample obtained from the individual comprises cancer cells that express HER2 on their cell surface at a level of IHC3+, as measured by IHC assay.

42. The method of claim 40, wherein a sample obtained from the individual comprises cancer cells that express HER2 on their cell surface at a level of IHC2+, as measured by IHC assay; and a sample obtained from the individual comprises cancer cells that exhibit HER2 gene amplification, as measured by ISH assay (ISH-positive).

43. The method of any one of claims 36-39, wherein the cancer is a HER2-low cancer.

44. The method of claim 43, wherein the cancer is HER2-low LA/mGC/GEJC.

45. The method of claim 43 or claim 44, wherein a sample obtained from the individual comprises cancer cells that express HER2 on their cell surface at a level of IHC1+, as measured by IHC assay.

46. The method of claim 44 or claim 45, wherein a sample obtained from the individual comprises cancer cells that express HER2 on their cell surface at a level of IHC2+, as measured by IHC assay; and a sample obtained from the individual comprises cancer cells that do not exhibit HER2 gene amplification, as measured by in situ hybridization (ISH) assay (ISH-negative).

47. The method of any one of claims 1-46, wherein the antibody-drug conjugate is administered to the individual at a dose of 1.0 mg/kg, 1.25 mg/kg, or 1.5 mg/kg.

48. The method of claim 47, wherein the antibody-drug conjugate is administered to the individual with a maximum dose of 150 mg.

49. The method of any one of claims 1-48, wherein the antibody-drug conjugate is administered intravenously to the individual.

50. The method of any one of claims 1-49, wherein the antibody-drug conjugate is administered to the individual every 2 weeks or every 14 days.

51. The method of any one of claims 47-50, wherein the antibody-drug conjugate is disitamab vedotin.

52. The method of any one of claims 1-51, wherein tucatinib is administered to the individual at a dose of between about 200 mg and about 300 mg.

53. The method of claim 52, wherein tucatinib is administered to the individual at a dose of 200 mg, 250 mg, or 300 mg.

54. The method of claim 53, wherein tucatinib is administered to the individual at a dose of 300 mg.

55. The method of any one of claims 1-54, wherein tucatinib is administered orally to the individual.

56. The method of any one of claims 1-55, wherein tucatinib is administered to the individual twice daily.

57. The method of any one of claims 1-2 and 17-46, wherein the antibody-drug conjugate is disitamab vedotin, and the method comprises:
(a) intravenously administering disitamab vedotin to the individual at 1.0 mg/kg, 1.25 mg/kg, or 1.5 mg/kg with a maximum dose of 150 mg on Days 1, 15, and 29 of a 6-week cycle; and
(b) orally administering tucatinib to the individual at 300 mg twice daily starting at Day 8 of the 6-week cycle.

58. The method of any one of claims 1-57, wherein administration of tucatinib and the anti-HER2 antibody-drug conjugate results in increased internalization of the antibody-drug conjugate by cells of the cancer, as compared to administration of the antibody-drug conjugate in the absence of tucatinib.

59. The method of any one of claims 1-58, wherein administration of tucatinib and the antibody-drug conjugate results in increased expression of HER2 by cells of the cancer, as compared to administration of the antibody-drug conjugate in the absence of tucatinib.

60. The method of claim 59, wherein administration of tucatinib and the antibody-drug conjugate results in increased total expression of HER2 by cells of the cancer, as compared to administration of the antibody-drug conjugate in the absence of tucatinib.

61. The method of claim 59 or claim 60, wherein administration of tucatinib and the anti-HER2 antibody-drug conjugate results in increased surface expression of HER2 by cells of the cancer, as compared to administration of the antibody-drug conjugate in the absence of tucatinib.

62. The method of any one of claims 1-61, wherein administration of tucatinib and the antibody-drug conjugate results in a complete response (CR) or partial response (PR) in the individual.

63. The method of any one of claims 1-62, wherein the individual is a human.

64. A composition comprising an anti-HER2 antibody-drug conjugate for use in a method of treating or preventing progression of cancer in an individual, wherein the method comprises administering an effective amount of tucatinib and the anti-HER2 antibody-drug conjugate according to the method of any one of claims 1-63.

65. A composition comprising an anti-HER2 antibody-drug conjugate and tucatinib for use in a method of treating or preventing progression of cancer in an individual, wherein the method comprises administering an effective amount of the composition according to the method of any one of claims 1-63.

66. A kit comprising tucatinib and an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

67. A kit comprising tucatinib and an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO: 11); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

68. The kit of claim 66 or claim 67, further comprising instructions for administering an effective amount of tucatinib and the antibody-drug conjugate to an individual in need thereof according to the method of any one of claims 1-63.

69. A kit comprising:
(a) an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO: 6); and
(b) instructions for administering an effective amount of the antibody-drug conjugate and tucatinib to an individual in need thereof according to the method of any one of claims 1-63.

70. A kit comprising:
(a) an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:11); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO: 6); and
(b) instructions for administering an effective amount of the antibody-drug conjugate and tucatinib to an individual in need thereof according to the method of any one of claims 1-63.
